(19) United States
(12) Patent Application Publication
(10) Pub. No.: US 2025/0053030 A1
(43) Pub. Date: Feb. 13, 2025

(54) ELECTRO-OPTIC FILM, COMPOUND, ELECTRO-OPTIC COMPOSITION, AND ELECTRO-OPTIC ELEMENT

(71) Applicant: Sumitomo Chemical Company, Limited, Chuo-ku, Tokyo (JP)

(72) Inventors: Eiji YOSHIKAWA, Osaka-shi, Osaka (JP); Takahiro SATO, Tsukuba-shi, Ibaraki (JP); Yuya YOSHIMOTO, Tsukuba-shi, Ibaraki (JP)

(21) Appl. No.: 18/711,337

(22) PCT Filed: Nov. 17, 2022
(86) PCT No.: PCT/JP2022/042674

§ 371 Date: May 17, 2024

(30) Foreign Application Priority Data
Dec. 7, 2021 (JP) 2021-198756

(51) Int. Cl. G02F 1/00; C07D 495/14; C07D 495/22; C07F 7/18

(52) U.S. Cl. CPC G02F 1/0018; C07D 495/14; C07D 495/22; C07F 7/1804

(57) ABSTRACT

Provided is an electro-optic film. The electro-optic film contains a compound (A) having a dipole moment of 19 to 31 debye. The electro-optic film has a maximum absorption wavelength (λ max) of 760 to 830 nm. A difference between the maximum absorption wavelength and an absorption wavelength on a longer wavelength side than the maximum absorption wavelength among absorption wavelengths exhibiting absorbance of a half value of maximum absorbance of the electro-optic film is 90 to 120 nm.

### TECHNICAL FIELD

The present invention relates to an electro-optic film, a compound, an electro-optic composition, and an electro-optic element.

### BACKGROUND ART

As an electro-optic (hereinafter, also abbreviated as “EO”) material applicable to a light control element (optical element) such as an optical modulator, an optical switch, an optical interconnect, an optoelectronic circuit, wavelength conversion, an electric field sensor, terahertz (THz) wave generation and detection, or an optical phased array, an inorganic ferroelectric EO material is conventionally used. However, the inorganic ferroelectric EO material has limitations in terms of high speed, miniaturization, and integration. Therefore, in order to achieve next-generation ultra-high-speed optical communication, a material capable of high-speed operation and capable of hybrid with silicon photonics is required.

An organic EO material has attracted attention from such a viewpoint. The organic EO material exhibits a large electro-optic effect as compared with the inorganic ferroelectric EO material, can operate at high speed, and can be miniaturized and integrated by hybrid with silicon photonics, and thus is expected as a material that performs next-generation optical communication.

A compound used for the organic EO material (hereinafter, also referred to as “EO compound”) has a structure in which a donor and an acceptor are linked to each other by a n conjugated bridge as a basic structure. In order to increase an EO coefficient of the EO material, it is known to adopt a donor having a high electron donating property and an acceptor having a high electron withdrawing property for the EO compound to increase the length of the n conjugated bridge. As the EO compound having such a structure, EO compounds having various structures have been reported (for example, Patent Documents 1 and 2 and Non-Patent Document 1).

### PRIOR ART DOCUMENTS

### Patent Documents

Patent Document 1: JP-A-2004-501159
Patent Document 2: WO-A-2019/151318

### Non-Patent Document

Non-Patent Document 1: Chem. Mater. 2008, 120, 6372-6377.

### SUMMARY OF THE INVENTION

### Problems to be Solved by the Invention

By the way, development of an EO compound and an electro-optic film (hereinafter, also referred to as “EO film”) containing the EO compound has been performed for the purpose of application in a C-band (wavelength: 1530 to 1565 nm) region used in long distance optical communication. Meanwhile, in recent years, it has been required to increase a speed of a middle-to-short distance optical interconnect, and for example, an O band region (wavelength: 1260 to 1360 nm) is used in a middle-to-short distance optical interconnect. In use of a conventional EO compound and an EO film containing the EO compound in the 0 band region, even when absorbance in the region is extremely low, an optical loss tends to easily occur, which may be an obstacle to optical communication. Such a tendency is more significant as the EO coefficient increases.

Therefore, an object of the present invention is to provide an electro-optic film capable of suppressing an optical loss in the O band region. Another object of the present invention is to provide a novel compound, and an electro-optic composition and an electro-optic film using such a compound. Still another object of the present invention is to provide an electro-optic element using the electro-optic film.

### Means for Solving the Problems

As a result of intensive studies in view of the above problems, the present inventors have found the following points and completed the present invention.
(1) In an electro-optic film, it is effective to use a compound in which a conjugate length of a structure in which a donor and an acceptor are linked to each other by a n conjugated bridge is in a predetermined range from a viewpoint of improving an EO coefficient.
(2) It can be estimated from a dipole moment of the compound that the conjugate length is in the predetermined range.
(3) In an electro-optic film containing a predetermined compound, a maximum absorption wavelength and a difference between the maximum absorption wavelength and an absorption wavelength on a longer wavelength side than the maximum absorption wavelength among absorption wavelengths exhibiting absorbance of a half value of maximum absorbance affect absorbance in then O band region.
(4) Furthermore, the maximum absorption wavelength and a long wavelength side half-value width are each in a predetermined range, whereby it is possible to suppress an optical loss in the O band region.

The present invention provides electro-optic films according to the following [1] to [5] and [8], a compound according to the following [6], an electro-optic composition according to the following [7], and an electro-optic element according to the following [9].

[1] An electro-optic film containing a compound (A) having a dipole moment of 19 to 31 debye, in which
a maximum absorption wavelength (λmax) is 760 to 830 nm, and
a difference between the maximum absorption wavelength and an absorption wavelength on a longer wavelength side than the maximum absorption wavelength among absorption wavelengths exhibiting absorbance of a half value of maximum absorbance is 90 to 120 nm.
   [2] The electro-optic film according to [1], in which the compound (A) is a compound represented by the following formula (1).

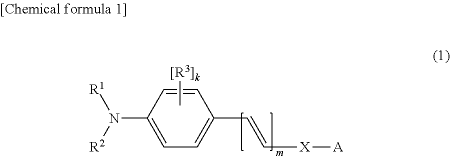

[In formula (1), X represents a bicyclic to pentacyclic divalent condensed ring group having two or more thiophene rings, and the divalent condensed ring group may have a substituent.

R¹ and R² each independently represent an alkyl group, a haloalkyl group, an acyloxyalkyl group, a trialkylsilyloxyalkyl group, an aryldialkylsilyloxyalkyl group, an alkyldiarylsilyloxyalkyl group, an aryl group, —R²¹—OH (R²¹ represents a divalent hydrocarbon group), —R²²—NH₂ (R²² represents a divalent hydrocarbon group), —R²³—SH (R²³ represents a divalent hydrocarbon group), or —R²⁴—NCO (R²⁴ represents a divalent hydrocarbon group). These groups may each have a crosslinkable group. R¹ and R² may be bonded to each other to form a ring together with atoms to which R¹ and R² are bonded.

R³ represents an alkyl group, an alkyloxy group, an aryl group, an aryloxy group, an aralkyloxy group, a trialkylsilyloxyalkyl group, an aryldialkylsilyloxyalkyl group, an alkyldiarylsilyloxyalkyl group, an alkenyloxy group, an alkynyloxy group, a hydroxy group, an amino group, a sulfanyl group, an isocyanate group, —R³¹—OH (R³¹ represents a divalent hydrocarbon group), —O—R³²—OH (R³² represents a divalent hydrocarbon group), —R³³—NH₂ (R³³ represents a divalent hydrocarbon group), —R³⁴—SH (R³⁴ represents a divalent hydrocarbon group), —R³⁵—NCO (R³⁵ represents a divalent hydrocarbon group), or —OC(═O)R⁴¹ (R⁴¹ represents a monovalent hydrocarbon group). These groups may each have a crosslinkable group. When there is a plurality of R³s, R³s may be the same as or different from each other. R³ may be bonded to R¹ or R² to form a ring together with atoms to which R³ and R¹ or R² are bonded.

k represents an integer of 0 to 4.

A represents any one of groups represented by the following formulas (a1), (a2), and (b1).

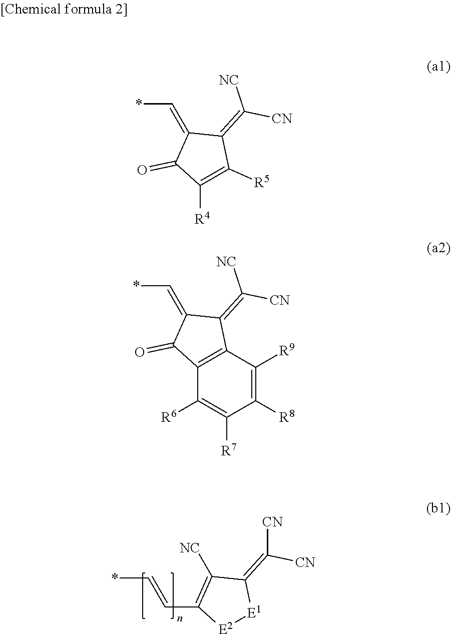

R⁴, R⁵, R⁶, R⁷, R⁸, and R⁹ each independently represent a hydrogen atom, a halogen atom, an alkyl group, a haloalkyl group, a cyano group, an aryl group, or a haloaryl group.

E¹ and E² each independently represent —C(R¹⁰)(R¹¹)—, —C(O)—, —O—, or —NR¹²—. Note that at least one of E¹ and E² is —O— or —NR¹²—. R¹⁰ and R¹¹ each independently represent a hydrogen atom, an alkyl group, a haloalkyl group, an aryl group, or a haloaryl group. R¹² represents a hydrogen atom or an alkyl group.

When A is a group represented by formula (a1) or (a2), m is 0.

When A is a group represented by formula (b1), m and n each independently represent 0 or 1. Note that m+n=1.]

[3] The electro-optic film according to [2], in which the compound represented by the above formula (1) is a compound represented by the following formula (2a2) or (2b1).

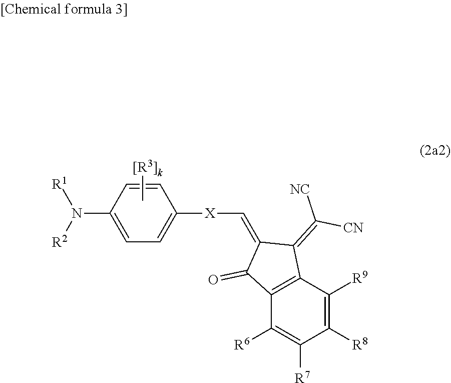

[In formula (2a2), X, R¹, R², R³, R⁶, R⁷, R⁸, R⁹, and k have the same meanings as those described above.]

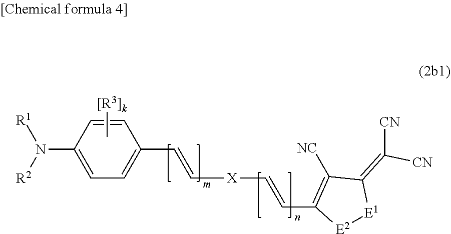

[In formula (2b1), X, R¹, R², R³, E¹, E², k, m, and n have the same meanings as those described above.]

[4] The electro-optic film according to [2] or [3], in which the compound represented by the above formula (1) is a compound represented by the following formula (2b1-1).

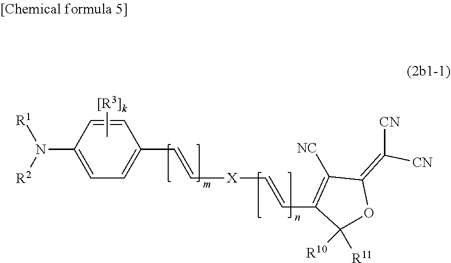

[In formula (2b1-1), X, R¹, R², R³, k, m, and n have the same meanings as those described above.

R¹⁰ and R¹¹ each independently represent a methyl group, a trifluoromethyl group, a phenyl group, or a pentafluorophenyl group.]

[5] The electro-optic film according to [2], in which the compound represented by the above formula (1) is a compound represented by the following formula (5).

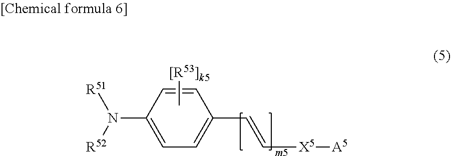

[In formula (5), X⁵ represents a bicyclic to tetracyclic divalent condensed ring group having two or more thiophene rings, and the divalent condensed ring group may have a substituent.

R⁵¹ and R⁵² each independently represent an alkyl group, a haloalkyl group, an acyloxyalkyl group, a trialkylsilyloxyalkyl group, an aryldialkylsilyloxyalkyl group, an alkyldiarylsilyloxyalkyl group, an aryl group, —R⁷¹—OH (R⁷?represents a divalent hydrocarbon group), —R⁷²—NH₂ (R⁷² represents a divalent hydrocarbon group), —R⁷³—SH (R⁷³ represents a divalent hydrocarbon group), or —R⁷⁴—NCO (R⁷⁴ represents a divalent hydrocarbon group). These groups may each have a crosslinkable group. R⁵¹ and R⁵² may be bonded to each other to form a ring together with atoms to which R⁵¹ and R⁵² are bonded.

R⁵³ represents an alkyl group or an aryl group. These groups may each have a crosslinkable group. When there is a plurality of R⁵³s, R⁵³s may be the same as or different from each other. R⁵³ may be bonded to R⁵¹ or R⁵² to form a ring together with atoms to which R⁵³ and R⁵¹ or R⁵² are bonded.

k5 represents an integer of 0 to 4.

A⁵ represents any one of groups represented by the following formulas (a51), (a52), and (b51).

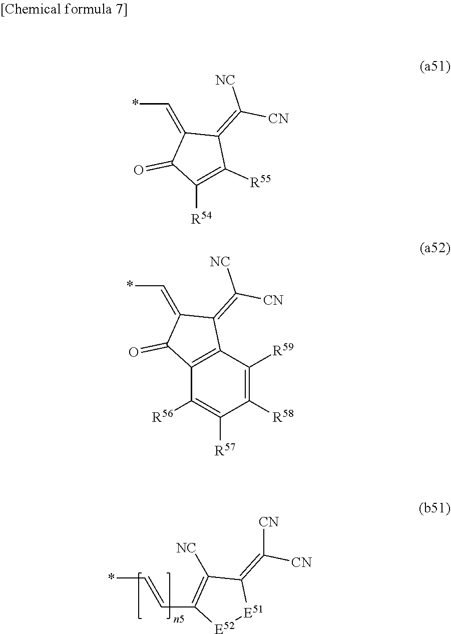

R⁵⁴, R⁵⁵, R⁵⁶, R⁵⁷, R⁵⁸, and R⁵⁹ each independently represent a hydrogen atom, a halogen atom, an alkyl group, a haloalkyl group, a cyano group, an aryl group, or a haloaryl group.

E⁵¹ and E⁵² each independently represent —C(R⁶⁰)(R⁶¹)—, —C(O)—, —O—, or —NR⁶²—. Note that at least one of E⁵¹ and E⁵² is —O— or —NR⁶²—. R⁶⁰ and R⁶¹ each independently represent a hydrogen atom, an alkyl group, a haloalkyl group, an aryl group, or a haloaryl group. R⁶² represents a hydrogen atom or an alkyl group.

When A⁵ is a group represented by formula (a51) or (a52), m5 is 0.

When A⁵ is a group represented by formula (b51), m5 and n5 each independently represent 0 or 1. Note that m5+n5=1.]

[6]A compound represented by the following formula (5).

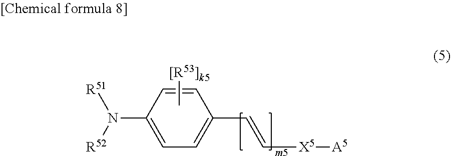

[In formula (5), X⁵ represents a bicyclic to tetracyclic divalent condensed ring group having two or more thiophene rings, and the divalent condensed ring group may have a substituent.

R⁵¹ and R⁵² each independently represent an alkyl group, a haloalkyl group, an acyloxyalkyl group, a trialkylsilyloxyalkyl group, an aryldialkylsilyloxyalkyl group, an alkyldiarylsilyloxyalkyl group, an aryl group, —R⁷¹—OH (R⁷?represents a divalent hydrocarbon group), —R⁷²—NH₂ (R⁷² represents a divalent hydrocarbon group), —R⁷³—SH (R⁷³ represents a divalent hydrocarbon group), or —R⁷⁴—NCO (R⁷⁴ represents a divalent hydrocarbon group). These groups may each have a crosslinkable group. R⁵¹ and R⁵² may be bonded to each other to form a ring together with atoms to which R⁵¹ and R⁵² are bonded.

R⁵³ represents an alkyl group or an aryl group. These groups may each have a crosslinkable group. When there is a plurality of R⁵³s, R⁵³s may be the same as or different from each other. R⁵³ may be bonded to R⁵¹ or R⁵² to form a ring together with atoms to which R⁵³ and R⁵¹ or R⁵² are bonded.

k5 represents an integer of 0 to 4.

A⁵ represents any one of groups represented by the following formulas (a51), (a52), and (b51).

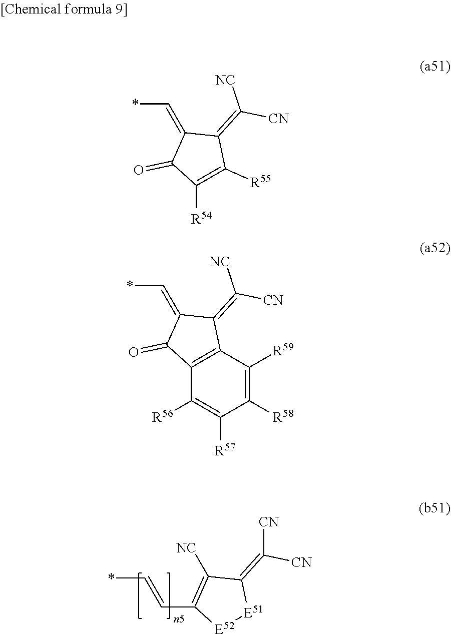

R⁵⁴, R⁵⁵, R⁵⁶, R⁵⁷, R⁵⁸, and R⁵⁹ each independently represent a hydrogen atom, a halogen atom, an alkyl group, a haloalkyl group, a cyano group, an aryl group, or a haloaryl group.

E⁵¹ and E⁵² each independently represent —C(R⁶⁰)(R⁶¹)—, —C(O)—, —O—, or —NR⁶²—. Note that at least one of E⁵¹ and E⁵² is —O— or —NR⁶²—. R⁶⁰ and R⁶¹ each independently represent a hydrogen atom, an alkyl group, a haloalkyl group, an aryl group, or a haloaryl group. R⁶² represents a hydrogen atom or an alkyl group.

When A⁵ is a group represented by formula (a51) or (a52), m5 is 0.

When A⁵ is a group represented by formula (b51), m5 and n5 each independently represent 0 or 1. Note that m5+n5=1.]

[7] An electro-optic composition containing the compound according to [6].
[8] An electro-optic film containing the compound according to [6].
[9] An electro-optic element including the electro-optic film according to any one of [1] to [5] or the electro-optic film according to [8].

### Effect of the Invention

The present invention provides an electro-optic film capable of suppressing an optical loss in an O band region (wavelength: 1260 to 1360 nm). The electro-optic film has a small optical loss α with respect to an electro-optic coefficient r33 in the O band region, and therefore is suitable for optical communication in the O band region.

The present invention also provide a novel compound, and an electro-optic composition and an electro-optic film using such a compound. Furthermore, the present invention provides an electro-optic element using the electro-optic film.

### MODE FOR CARRYING OUT THE INVENTION

Hereinafter, a preferred embodiment of the present invention will be described in detail.

In the present specification, “long wavelength side half-value width” means a difference between a maximum absorption wavelength and an absorption wavelength on a longer wavelength side than the maximum absorption wavelength among absorption wavelengths exhibiting absorbance of a half value of maximum absorbance.

### Electro-Optic Film

An EO film of the present embodiment contains a compound (A).

### Compound (A)

The compound (A) is a compound having a dipole moment of 19 to 31 debye. The compound (A) usually has a structure in which a donor and an acceptor are linked to each other by a n conjugated bridge.

When the dipole moment of the compound (A) is 19 debye or more, numerical values such as a hyperpolarizability β of an EO compound and an electro-optic coefficient r33 of the EO film tend to be sufficiently high. When the dipole moment of the compound (A) is 31 debye or less, it tends to be easy to obtain an EO film in which an increase in value of a wavelength of an absorption spectrum is suppressed and an optical loss in an O band region is small. The dipole moment of the compound (A) is preferably 20 debye or more, more preferably 22 debye or more, still more preferably 24 debye or more, and preferably 30 debye or less, more preferably 29 debye or less, still more preferably 27 debye or less.

A numerical value of the dipole moment of the compound (A) can be increased, for example, by increasing an electron donating property of a donor and/or an electron withdrawing property of an acceptor.

The dipole moment μ of the compound (A) can be calculated, for example, by Gaussian 09 which is a quantum chemical calculation program manufactured by Gaussian. More specifically, the dipole moment μ of the compound (A) can be calculated by performing structure optimization calculation by pcm calculation (designating chloroform as a solvent) under an M062X/6-31+g(d) condition.

The compound (A) is preferably a compound represented by the following formula (1). The compound represented by formula (1) has a predetermined polycyclic condensed ring group and a predetermined linking group linking the polycyclic condensed ring group. By having a predetermined polycyclic condensed ring group, the compound represented by formula (1) has a high hyperpolarizability. In addition, by having a predetermined polycyclic condensed ring group and a predetermined linking group linking the polycyclic condensed ring group, the compound represented by formula (1) can suppress a multimerization reaction (for example, a Diels-Alder reaction) between the molecules by heating, and has excellent heat resistance.

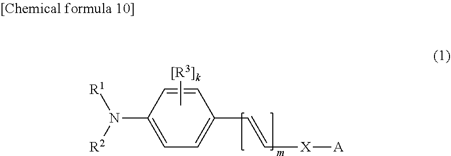

In formula (1), X represents a bicyclic to pentacyclic divalent condensed ring group having two or more thiophene rings, and the divalent condensed ring group may have a substituent.

The divalent condensed ring group as X has two or more thiophene rings. The number of thiophene rings is preferably 2 to 5, more preferably 2 to 4, and still more preferably 2 or 3. Note that, in a ring-condensed thiophene in which thiophenes are ring-condensed, the number of ring-condensed thiophenes is the number of thiophene rings. For example, in thienothiophene in which two thiophenes are ring-condensed, the number of thiophene rings is counted as 2.

The divalent condensed ring group as X is bicyclic to pentacyclic, and is preferably bicyclic to pentacyclic, more preferably bicyclic to tetracyclic, and still more preferably bicyclic or tricyclic from a viewpoint of easily adjusting a dipole moment in a predetermined range. When the number of condensed rings is small (for example, tetracyclic or less or tricyclic or less), the divalent condensed ring group preferably has no benzene ring.

The divalent condensed ring group as X preferably has at least one selected from the group consisting of an sp3 carbon atom, a nitrogen atom, and a silicon atom as a constituent element. That is, the divalent condensed ring group preferably has at least one group selected from the group consisting of a group represented by —C(R^{A})(R^{B})— in the ring, a group represented by —N(R^{C})— in the ring, and a group represented by —Si(R^{D})(R^{E})— in the ring. A carbon atom in —C(R^{A})(R^{B})— may be a tertiary carbon atom in which one of R^{A} and R^{B} is an alkyl group or the like and the other is a hydrogen atom, or a quaternary carbon atom in which both R^{A} and R^{B} are alkyl groups and the like, and is preferably a quaternary carbon atom.

The number of sp3 carbon atoms in the divalent condensed ring group as X is preferably 1 to 3, more preferably 1 or 2, and still more preferably 1. The number of nitrogen atoms in the divalent condensed ring group as X is preferably 1 to 3, more preferably 1 or 2, and still more preferably 1.

The number of silicon atoms in the divalent condensed ring group as X is preferably 1 to 3, more preferably 1 or 2, and still more preferably 1.

R^{A}, R^{B}, R^{C}, R^{D}, and R^{E} each independently represent a hydrogen atom, an alkyl group, a cycloalkyl group, an alkyloxy group, a cycloalkyloxy group, an alkylthio group, a cycloalkylthio group, an aryl group, or a monovalent heterocyclic group. These groups may each have a substituent.

The alkyl group as each of R^{A}, R^{B}, R^{C}, R^{D}, and R^{E} may be linear or branched. The number of carbon atoms of the alkyl group is usually 1 to 30 without including the number of carbon atoms of a substituent.

Specific examples of the alkyl group include a methyl group, an ethyl group, a propyl group, an isopropyl group, a butyl group, an isobutyl group, a sec-butyl group, a tert-butyl group, a pentyl group, an isopentyl group, a 2-methylbutyl group, a 1-methylbutyl group, a hexyl group, an isohexyl group, a 3-methylpentyl group, a 2-methylpentyl group, a 1-methylpentyl group, a heptyl group, an octyl group, an isooctyl group, a 2-ethylhexyl group, a 3,7-dimethyloctyl group, a nonyl group, a decyl group, an undecyl group, a dodecyl group, a tetradecyl group, a hexadecyl group, an octadecyl group, and an eicosyl group.

The number of carbon atoms of the cycloalkyl group as each of R^{A}, R^{B}, R^{C}, R^{D}, and R^{E} is usually 3 to 30 without including the number of carbon atoms of a substituent. Specific examples of the cycloalkyl group include a cyclopropyl group, a cyclopentyl group, a cyclohexyl group, and an adamantyl group.

The alkyl group in the alkyloxy group as each of R^{A}, R^{B}, R^{C}, R^{D}, and R^{E} may be linear or branched. The number of carbon atoms of the alkyloxy group is usually 1 to 30 without including the number of carbon atoms of a substituent. Specific examples of the alkyloxy group include a methoxy group, an ethoxy group, a propoxy group, an isopropoxy group, a butoxy group, an isobutoxy group, a tert-butoxy group, a pentyloxy group, a hexyloxy group, a heptyloxy group, an octyloxy group, a 2-ethylhexyloxy group, a nonyloxy group, a decyloxy group, a 3,7-dimethyloctyloxy group, and a lauryloxy group.

The number of carbon atoms of the cycloalkyloxy group as each of R^{A}, R^{B}, R^{C}, R^{D}, and R^{E} is usually 3 to 30 without including the number of carbon atoms of a substituent. Specific examples of the cycloalkyloxy group include a cyclopropyloxy group, a cyclopentyloxy group, a cyclohexyloxy group, and an adamantyloxy group.

The alkyl group in the alkylthio group as each of R^{A}, R^{B}, R^{C}, R^{D}, and R^{E} may be linear or branched. The number of carbon atoms of the alkylthio group is usually 1 to 30 without including the number of carbon atoms of a substituent. Specific examples of the alkylthio group include a methylthio group, an ethylthio group, a propylthio group, an isopropylthio group, a butylthio group, an isobutylthio group, a tert-butylthio group, a pentylthio group, a hexylthio group, a heptylthio group, an octylthio group, a 2-ethylhexylthio group, a nonylthio group, a decylthio group, a 3,7-dimethyloctylthio group, and a laurylthio group.

The number of carbon atoms of the cycloalkylthio group as each of R^{A}, R^{B}, R^{C}, R^{D}, and R^{E} is usually 3 to 30 without including the number of carbon atoms of a substituent. Specific examples of the cycloalkylthio group include a cyclopropylthio group, a cyclopentylthio group, a cyclohexylthio group, and an adamantylthio group.

The number of carbon atoms of the aryl group as each of R^{A}, R^{B}, R^{C}, R^{D}, and R^{E} is usually 6 to 30 without including the number of carbon atoms of a substituent. Specific examples of the aryl group include a phenyl group, a 1-naphthyl group, a 2-naphthyl group, a 1-anthracenyl group, a 2-anthracenyl group, a 9-anthracenyl group, a 1-pyrenyl group, a 2-pyrenyl group, a 4-pyrenyl group, a 2-fluorenyl group, a 3-fluorenyl group, a 4-fluorenyl group, a 2-phenylphenyl group, a 3-phenylphenyl group, and a 4-phenylphenyl group.

The number of carbon atoms of the monovalent heterocyclic group as each of R^{A}, R^{B}, R^{C}, R^{D}, and R^{E} is usually 2 to 30 without including the number of carbon atoms of a substituent. Examples of the monovalent heterocyclic group include a group obtained by removing, from a heterocyclic compound such as furan, thiophene, pyrrole, pyrroline, pyrrolidine, oxazole, isoxazole, thiazole, isothiazole, imidazole, imidazoline, imidazolidine, pyrazole, pyrazoline, prazolidine, furazan, triazole, thiadiazole, oxadiazole, tetrazole, pyran, pyridine, piperidine, thiopyran, pyridazine, pyrimidine, pyrazine, piperazine, morpholine, triazine, benzofuran, isobenzofuran, benzothiophene, indole, isoindole, indolizine, indoline, isoindoline, chromene, chroman, isochromane, benzopyran, quinoline, isoquinoline, quinolidine, benzimidazole, benzothiazole, indazole, naphthyridine, quinoxaline, quinazoline, quinazolidine, cinnoline, phthalazine, purine, pteridine, carbazole, xanthene, phenanthridine, acridine, β-carboline, perimidine, phenanthroline, thianthrene, phenoxathiine, phenoxazine, phenothiazine, or phenazine, one hydrogen atom directly bonded to a carbon atom constituting the ring.

In the present specification, examples of the substituent include a halogen atom (a fluorine atom, a chlorine atom, a bromine atom, an iodine atom, or the like), a cyano group, an alkyl group, an alkyloxy group, a haloalkyl group, an aryl group, and a monovalent heterocyclic group.

Each of R^{A}, R^{B}, R^{C}, R^{D}, and R^{E} is preferably an alkyl group having 1 to 30 carbon atoms, a cycloalkyl group having 3 to 30 carbon atoms, or an aryl group having 6 to 30 carbon atoms from a viewpoint of maintaining heat resistance and suppressing aggregation between molecules.

Examples of the divalent condensed ring group as X include groups represented by formulas (X-1) to (X-38).

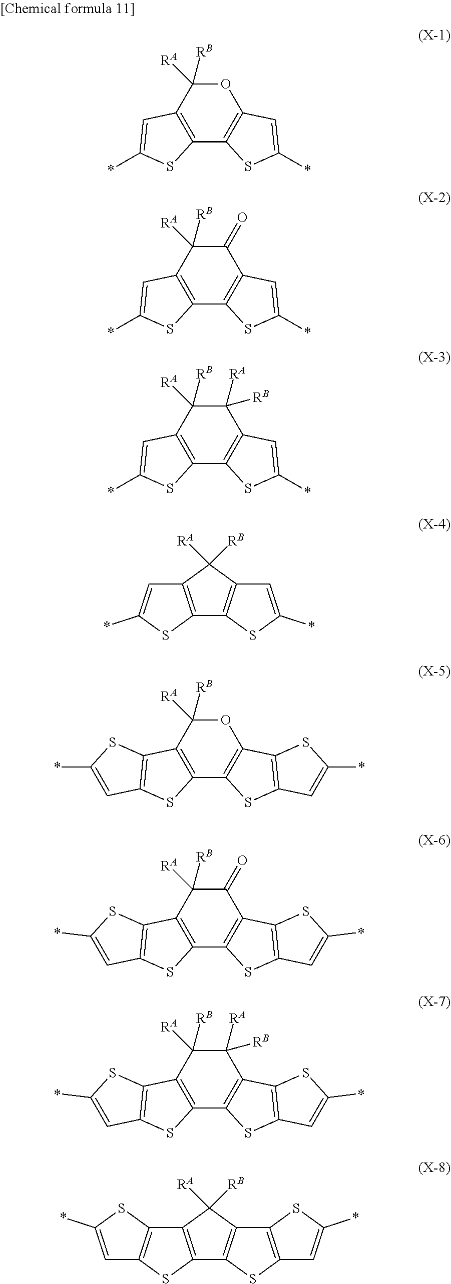

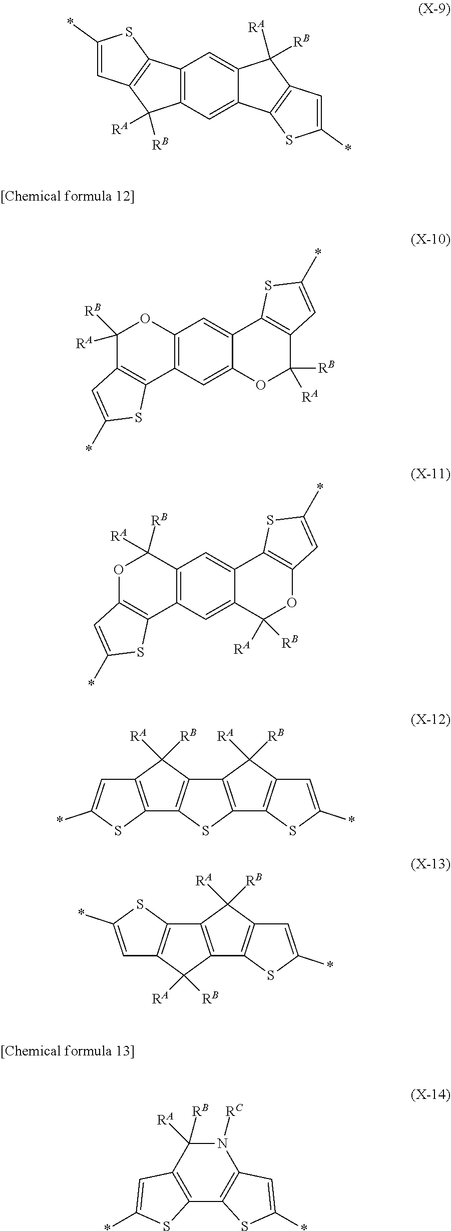

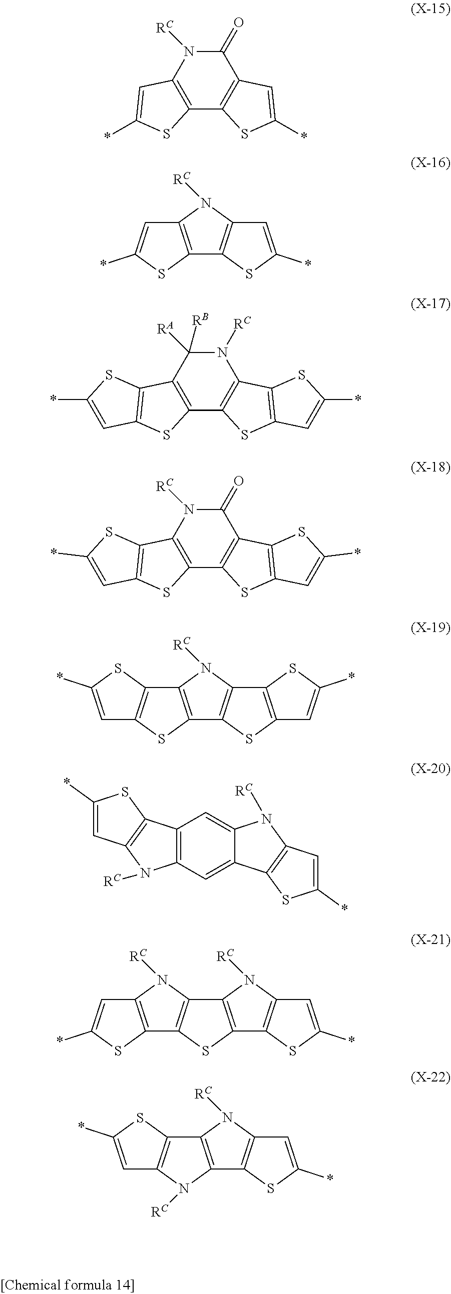

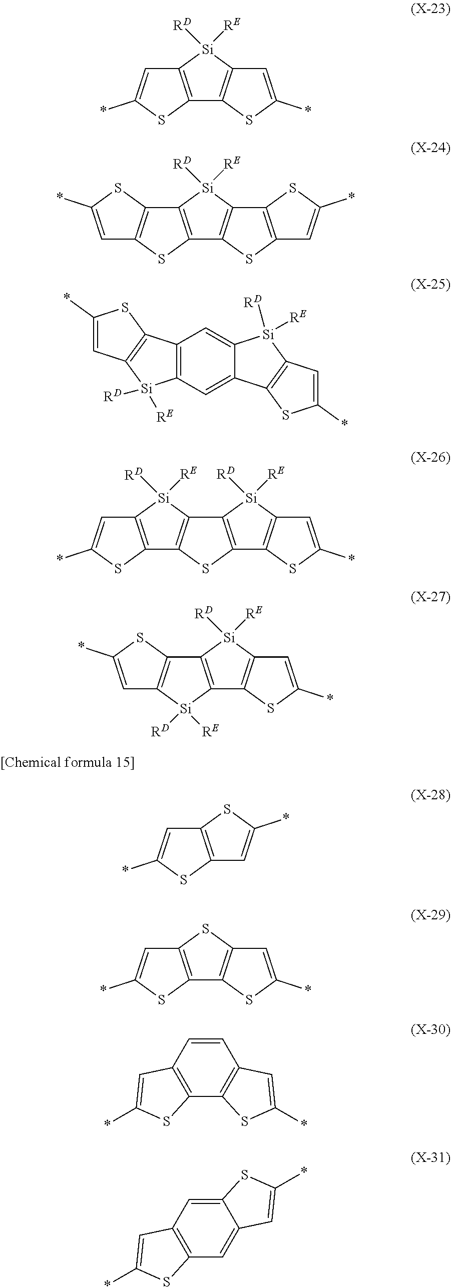

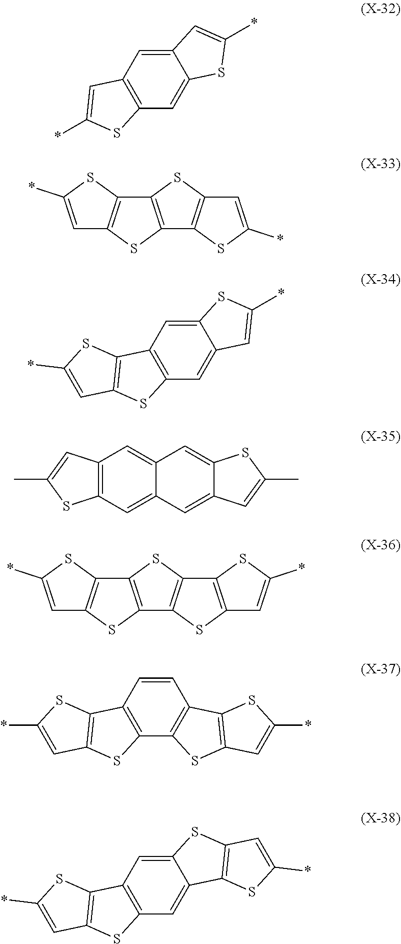

The divalent condensed ring group as X is preferably a group represented by any one of formulas (X-1) to (X-4), formulas (X-13) to (X-16), formula (X-22), formula (X-23), and formulas (X-27) to (X-35), more preferably a group represented by any one of formulas (X-1) to (X-4), formulas (X-14) to (X-16), formula (X-23), and formulas (X-28) to (X-32), still more preferably a group represented by any one of formulas (X-1) to (X-4), formulas (X-14) to (X-16), formula (X-23), formula (X-28), and formula (X-29), particularly preferably a group represented by formula (X-1), formula (X-4), formula (X-16), formula (X-28), or formula (X-29), and most preferably a group represented by formula (X-1), formula (X-4), or formula (X-28) from a viewpoint of easily adjusting the dipole moment in a predetermined range

By having such a divalent polycyclic condensed ring group as X, the compound represented by formula (1) is a compound having high linearity and flatness, and can be suitably used as an EO compound.

R¹ and R² each independently represent an alkyl group, a haloalkyl group, an acyloxyalkyl group, a silyloxyalkyl group, an aryl group, —R²¹—OH (in which R²¹ represents a divalent hydrocarbon group), —R²²—NH₂ (in which R²² represents a divalent hydrocarbon group), —R²³—SH (in which R²³ represents a divalent hydrocarbon group), or —R²⁴—NCO (in which R²⁴ represents a divalent hydrocarbon group). These groups may each have a crosslinkable group. R¹ and R² may be bonded to each other to form a ring together with atoms to which R¹ and R² are bonded.

Examples of the alkyl group as each of R¹ and R² include the alkyl groups exemplified for R^{A}, R^{B}, R^{C}, R^{D}, and R^{E}. The number of carbon atoms of the alkyl group is preferably 1 to 20, more preferably 1 to 10, and still more preferably 1 to 5.

The haloalkyl group as each of R¹ and R² is an alkyl group having one or more halogen atoms (for example, a fluorine atom, a chlorine atom, a bromine atom, or an iodine atom) as substituents. The number of carbon atoms of the haloalkyl group is preferably 1 to 20, more preferably 1 to 10, and still more preferably 1 to 5. Specific examples of the haloalkyl group include a fluoromethyl group, a difluoromethyl group, a trifluoromethyl group, a 2-fluoroethyl group, a 1,2-difluoroethyl group, a chloromethyl group, a 2-chloroethyl group, a 1,2-dichloroethyl group, a bromomethyl group, a 2-bromoethyl group, a 1-bromopropyl group, a 2-bromopropyl group, a 3-bromopropyl group, and an iodomethyl group.

Examples of the acyloxyalkyl group as each of R¹ and R² include an alkyl group having one or more acyloxy groups as substituents. The number of carbon atoms of the acyloxyalkyl group is preferably 2 to 20, more preferably 3 to 10, and still more preferably 3 to 7.

Examples of the trialkylsilyloxyalkyl group, the aryldialkylsilyloxyalkyl group, and the alkyldiarylsilyloxyalkyl group as each of R¹ and R² include an alkyl group having one or more trialkylsilyloxy groups as substituents, an alkyl group having one or more aryldialkylsilyloxy groups as substituents, and an alkyl group having one or more alkyldiarylsilyloxy groups as substituents. The number of carbon atoms of each of the trialkylsilyloxyalkyl group, the aryldialkylsilyloxyalkyl group, and the alkyldiarylsilyloxyalkyl group is preferably 5 to 25, more preferably 10 to 22, and still more preferably 12 to 20.

Examples of the aryl group as each of R¹ and R² include the aryl groups exemplified for R^{A}, R^{B}, R^{C}, R^{D}, and R^{E}. The number of carbon atoms of the aryl group is preferably 6 to 20, and more preferably 6 to 10.

Examples of the divalent hydrocarbon group as each of R²¹, R²², R²³, and R²⁴ in each of R¹ and R² include an alkanediyl group and a cycloalkanediyl group. Specific examples of the alkanediyl group include: a linear alkanediyl group such as a methylene group, an ethylene group, a trimethylene group, a tetramethylene group, a pentamethylene group, a hexamethylene group, an octamethylene group, a decamethylene group, or a dodecamethylene group; and a branched alkanediyl group such as a propylene group, an isopropylene group, an isobutylene group, a 2-methyltrimethylene group, an isopentylene group, an isohexylene group, an isooctylene group, a 2-ethylhexylene group, or an isodecylene group. Specific examples of the cycloalkanediyl group include a cyclopropylene group, a cyclopentylene group, a cyclohexylene group, and a cyclododecylene group. The number of carbon atoms of the alkanediyl group is preferably 1 to 20, more preferably 1 to 10, and still more preferably 1 to 5. The number of carbon atoms of the cycloalkanediyl group is preferably 3 to 20.

Each of R¹ and R² is preferably an alkyl group having 1 to 10 carbon atoms, an acyloxyalkyl group having 3 to 10 carbon atoms, a silyloxyalkyl group having 5 to 10 carbon atoms, an aryl group having 6 to 10 carbon atoms, —R²¹—OH (R²¹ represents an alkanediyl group having 1 to 10 carbon atoms), —R²²—NH₂ (R²² represents an alkanediyl group having 1 to 10 carbon atoms), —R²³—SH (R²³ represents an alkanediyl group having 1 to 10 carbon atoms), or —R²⁴—NCO (in which R²⁴ represents an alkanediyl group having 1 to 10 carbon atoms), and more preferably an alkyl group having 1 to 5 carbon atoms, an acyloxyalkyl group having 3 to 7 carbon atoms, a silyloxyalkyl group having 6 to 9 carbon atoms, —R²¹—OH (R²¹ represents an alkanediyl group having 1 to 5 carbon atoms), —R²²—NH₂ (R²² represents an alkanediyl group having 1 to 5 carbon atoms), —R²³—SH (R²³ represents an alkanediyl group having 1 to 5 carbon atoms), or —R²⁴—NCO (in which R²⁴ represents an alkanediyl group having 1 to 5 carbon atoms) from a viewpoint of exhibiting excellent EO characteristics.

Each of R¹ and R² may have a crosslinkable group. The crosslinkable group means a group that reacts with an identical or different group of another molecule located in the vicinity by irradiation with heat and/or active energy rays to generate a novel chemical bond. Examples of the crosslinking group include: a radically polymerizable group such as a (meth)acryloyloxy group or a styryl group (vinylphenyl group); and a Diels-Alder polymerizable group that reacts with a dienophile, such as an anthracenyl group or a benzocyclobutenyl group.

R¹ and R² may be bonded to each other to form a ring together with atoms to which R¹ and R² are bonded. When R¹ and R² are bonded to each other to form a ring, the ring is preferably a 5-membered ring or a 6-membered ring from a viewpoint of ensuring stability. The ring is preferably an aliphatic ring.

R³ represents an alkyl group, an alkyloxy group, an aryl group, an aryloxy group, an aralkyloxy group, a silyloxyalkyl group, an alkenyloxy group, an alkynyloxy group, a hydroxy group, an amino group, a sulfanyl group, an isocyanate group, —R³¹—OH (R³¹ represents a divalent hydrocarbon group), —O—R³²—OH (R³² represents a divalent hydrocarbon group), —R³³—NH₂ (R³³ represents a divalent hydrocarbon group), —R³⁴—SH (R³⁴ represents a divalent hydrocarbon group), —R³⁵—NCO (R³⁵ represents a divalent hydrocarbon group), or —OC(═O)R⁴¹ (R⁴¹ represents a monovalent hydrocarbon group). These groups may each have a crosslinkable group. When there is a plurality of R³s, R³s may be the same as or different from each other. R³ may be bonded to R¹ or R² to form a ring together with atoms to which R³ and R¹ or R² are bonded.

Examples of the alkyl group and the alkyloxy group as R³ include the alkyl groups and the alkyloxy groups exemplified for R^{A}, R^{B}, R^{C}, R^{D}, and R^{E}. The number of carbon atoms of the alkyl group is preferably 1 to 20, more preferably 1 to 10, and still more preferably 1 to 5.

Examples of the aryl group and the aryl group of the aryloxy group as R³ include the aryl groups exemplified for R^{A}, R^{B}, R^{C}, R^{D}, and R^{E}. The number of carbon atoms of the aryl group is preferably 6 to 20, and more preferably 6 to 10.

Examples of the aralkyl group of the aralkyloxy group as R³ include an alkyl group having one or more aralkyl groups as substituents. Specific examples of the aralkyl group include a benzyl group, a 1-phenylethyl group, a phenethyl group, a 1-naphthylmethyl group, a 2-naphthylmethyl group, a 1-naphthylethyl group, and a 2-naphthylethyl group.

Examples of the trialkylsilyloxyalkyl group, the aryldialkylsilyloxyalkyl group, and the alkyldiarylsilyloxyalkyl group as R³ include the trialkylsilyloxyalkyl groups, the aryldialkylsilyloxyalkyl groups, and the alkyldiarylsilyloxyalkyl groups exemplified for R¹ and R².

Examples of the alkenyl group of the alkenyloxy group as R³ include an alkenyl group having 2 to 20 carbon atoms. Specific examples of the alkenyl group include an ethenyl group, a 1-propenyl group, a 2-propenyl group, a 1-methylethenyl group, a 1-butenyl group, a 2-butenyl group, a 3-butenyl group, a 1-methyl-1-propenyl group, a 1-methyl-2-propenyl group, a 2-methyl-1-propenyl group, and a 2-methyl-2-propenyl group.

Examples of the alkynyl group of the alkynyloxy group as R³ include an alkynyl group having 3 to 20 carbon atoms. Specific examples of the alkynyl group include a 2-propynyl group, a 1-methyl-2-propynyl group, a 1,1-dimethyl-2-propynyl group, a 2-butynyl group, a 3-butynyl group, a 1-pentynyl group, a 2-pentynyl group, a 3-pentynyl group, and a 4-pentynyl group.

In R³, examples of the divalent hydrocarbon group as each of R³¹, R³², R³³, R³⁴, and R³⁵ include the divalent hydrocarbon groups exemplified for R²¹, R²², R²³, and R²⁴.

In R³, examples of the monovalent hydrocarbon group as R⁴¹ include the alkyl groups and the cycloalkyl groups exemplified for R^{A}, R^{B}, R^{C}, R^{D}, and R^{E}.

k represents an integer of 0 to 4. k is preferably 0 or 1, and more preferably 0.

R³ may have a crosslinkable group. Examples of the crosslinkable group include the crosslinkable groups exemplified for R¹ and R².

R³ may be bonded to R¹ or R² to form a ring together with atoms to which R³ and R¹ or R² are bonded. When R³ is bonded to R¹ or R² to form a ring, the ring is preferably a 5-membered ring or a 6-membered ring from a viewpoint of ensuring stability. The ring is preferably an aliphatic ring.

A is a group having an acceptor structure, and represents any one of groups represented by the following formulas (a1), (a2), and (b1).

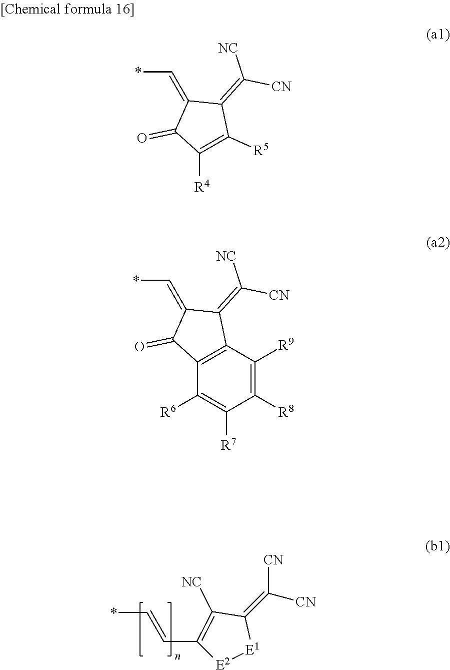

R⁴, R⁵, R⁶, R⁷, R⁸, and R⁹ each independently represent a hydrogen atom, a halogen atom, an alkyl group, a haloalkyl group, a cyano group, an aryl group, or a haloaryl group.

Examples of the halogen atom include a fluorine atom, a chlorine atom, a bromine atom, and an iodine atom. The halogen atom is preferably a fluorine atom.

Examples of the alkyl group, the haloalkyl group, and the aryl group as each of R⁴, R⁵, R⁶, R⁷, R⁸, and R⁹ include the alkyl groups, the haloalkyl groups, and the aryl groups exemplified for R¹ and R². The alkyl group is preferably a methyl group. The haloalkyl group is preferably a trifluoromethyl group. The alkyl group is preferably an aryl group.

The haloaryl group as each of R⁴, R⁵, R⁶, R⁷, R⁸, and R⁹ is an aryl group having one or more halogen atoms (for example, a fluorine atom, a chlorine atom, a bromine atom, or an iodine atom, preferably a fluorine atom) as substituents. The haloaryl group is preferably a pentafluorophenyl group.

E¹ and E² each independently represent —C(R¹⁰)(R¹¹)—, —C(O)—, —O—, or —NR¹²—. Note that at least one of E¹ and E² is —O— or —NR¹²—.

R¹⁰ and R¹¹ each independently represent a hydrogen atom, an alkyl group, a haloalkyl group, an aryl group, or a haloaryl group. Examples of the alkyl group, the haloalkyl group, the aryl group, and the haloaryl group as each of R¹⁰ and R¹¹ include the alkyl groups, the haloalkyl groups, the aryl groups, and the haloaryl groups exemplified for R⁴, R⁵, R⁶, R⁷, R⁸, and R⁹.

R¹² represents a hydrogen atom or an alkyl group. Examples of the alkyl group include the alkyl groups exemplified for R¹ and R².

When A is a group represented by formula (a1) or (a2), m is 0.

When A is a group represented by formula (b1), m and n each independently represent 0 or 1. Note that m+n=1. By satisfying such conditions, the compound represented by formula (1) can suppress a multimerization reaction (for example, a Diels-Alder reaction) between the molecules by heating, and has excellent heat resistance. m is preferably 0, and n is preferably 1. When m is 0 and n is 1, the multimerization reaction between the molecules by heating can be further suppressed.

The compound represented by formula (1) is preferably a compound represented by formula (2a1), a compound represented by formula (2a2), or a compound represented by formula (2b1), more preferably a compound represented by formula (2a2) or a compound represented by formula (2b1), and still more preferably a compound represented by formula (2b1).

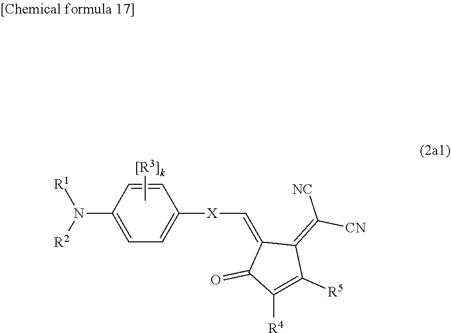

In formula (2a1), X, R¹, R², R³, R⁴, R⁵, and k have the same meanings as those described above.

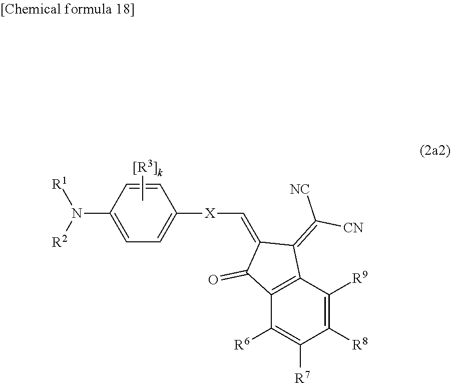

In formula (2a2), X, R¹, R², R³, R⁶, R⁷, R³, R⁹, and k have the same meanings as those described above.

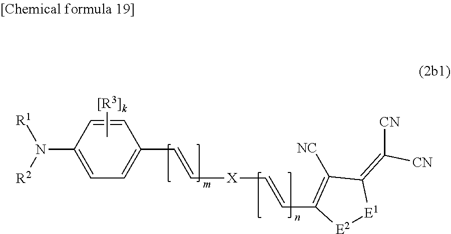

In formula (2b1), X, R¹, R², R³, E¹, E², k, m, and n have the same meanings as those described above.

The compound represented by formula (2b1) is preferably a compound represented by formula (2b1-1) or a compound represented by formula (2b1-2), and more preferably a compound represented by formula (2b1-1).

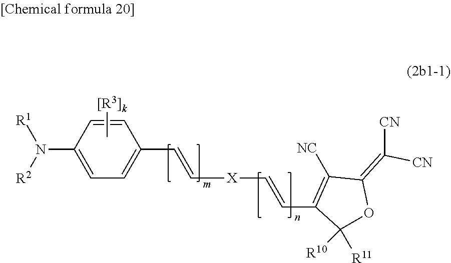

In formula (2b1-1), X, R¹, R², R³, k, m, and n have the same meanings as those described above.

R¹⁰ and R¹¹ each independently represent a methyl group, a trifluoromethyl group, a phenyl group, or a pentafluorophenyl group.

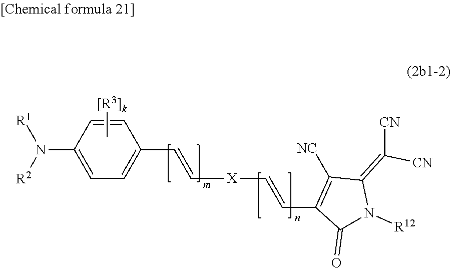

In formula (2b1-2), X, R¹, R², R³, R¹², k, m, and n have the same meanings as those described above.

The compound represented by formula (2b1) (or the compound represented by formula (2b1-1)) is preferably a compound represented by formula (2b1-1a).

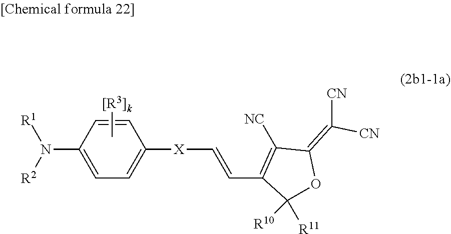

In formula (2b1-1a), X, R¹, R², R³, R¹⁰, R¹¹, and k have the same meanings as those described above.

The compound represented by formula (1) is preferably a compound represented by the following formula (5).

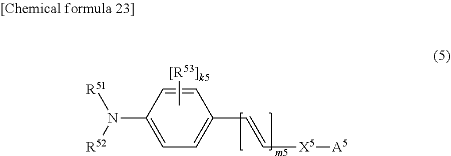

In formula (5), X⁵ represents a bicyclic to tetracyclic divalent condensed ring group having two or more thiophene rings, and the divalent condensed ring group may have a substituent. A preferred range of the divalent condensed ring group as X⁵ may be similar to the preferred range of the divalent condensed ring group as X described above.

R⁵¹ and R⁵² each independently represent an alkyl group, a haloalkyl group, an acyloxyalkyl group, a trialkylsilyloxyalkyl group, an aryldialkylsilyloxyalkyl group, an alkyldiarylsilyloxyalkyl group, an aryl group, —R⁷¹—OH (R⁷?represents a divalent hydrocarbon group), —R⁷²—NH₂ (R⁷² represents a divalent hydrocarbon group), —R⁷³—SH (R⁷³ represents a divalent hydrocarbon group), or —R⁷⁴—NCO (R⁷⁴ represents a divalent hydrocarbon group). These groups may each have a crosslinkable group. R⁵¹ and R⁵² may be bonded to each other to form a ring together with atoms to which R⁵¹ and R⁵² are bonded. R⁵¹, R⁵², R⁷¹, R⁷², R⁷³, and R⁷⁴ have the same meanings as R¹, R², R²¹, R²², R²³, and R²⁴ described above, respectively.

R⁵³ represents an alkyl group or an aryl group. These groups may each have a crosslinkable group. When there is a plurality of R⁵³s, R⁵³s may be the same as or different from each other. R⁵³ may be bonded to R⁵¹ or R⁵² to form a ring together with atoms to which R⁵³ and R⁵¹ or R⁵² are bonded. The alkyl group as R⁵³ has the same meaning as the alkyl group as R³ described above.

The aryl group as R⁵³ has the same meaning as the aryl group as R³ described above.

k5 represents an integer of 0 to 4. k5 has the same meaning as k described above.

A⁵ represents any one of groups represented by the following formulas (a51), (a52), and (b51).

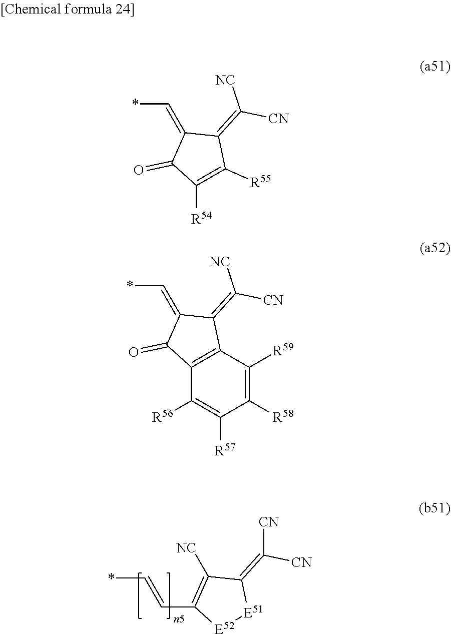

R⁵⁴, R⁵⁵, R⁵⁶, R⁵⁷, R⁵⁸, and R⁵⁹ each independently represent a hydrogen atom, a halogen atom, an alkyl group, a haloalkyl group, a cyano group, an aryl group, or a haloaryl group.

E⁵¹ and E⁵² each independently represent —C(R⁶⁰)(R⁶¹)—, —C(O)—, —O—, or —NR⁶²—. Note that at least one of E⁵¹ and E⁵² is —O— or —NR⁶²—. R⁶⁰ and R⁶¹ each independently represent a hydrogen atom, an alkyl group, a haloalkyl group, an aryl group, or a haloaryl group. R⁶² represents a hydrogen atom or an alkyl group.

When A⁵ is a group represented by formula (a51) or (a52), m5 is 0.

When A⁵ is a group represented by formula (b51), m5 and n5 each independently represent 0 or 1. Note that m5+n5=1.

A⁵, a group represented by formula (a51), a group represented by formula (a52), a group represented by formula (b51), R⁵⁴, R⁵⁵, R⁵⁶, R⁵⁷, R⁵⁸, R⁵⁹, E⁵¹, E⁵², R⁶⁰, R⁶¹, R⁶², m5, and n5 have the same meanings as the above A, group represented by formula (a1), group represented by formula (a2), group represented by formula (b1), R⁴, R⁵, R⁶, R⁷, R⁸, R⁹, E¹, E², R¹⁰, R¹¹, R¹², R⁶¹, m, and n, respectively.

In the compound represented by formula (1), a distance (hereinafter, also referred to as “distance CN”) between a carbon atom of A directly bonded to X and a nitrogen atom bonded to R¹ and R² is preferably 10.0 to 21.0 Å. Note that 1 Å means 1×10⁻¹⁰ m. The distance CN is more preferably 10.5 to 20.0 Å, and still more preferably 11.0 to 19.0 Å because a dipole moment as the compound (A) (the compound represented by formula (1) or the compound represented by formula (5)) can be easily adjusted in a predetermined range.

The distance CN can be calculated, for example, by performing structure optimization calculation by pcm calculation (designating chloroform as a solvent) under a condition of M062X/6-31+g(d) similarly to the method used for calculating a dipole moment μ of the compound (A). The distance CN can also be directly measured by, for example, X-ray crystal structure analysis.

A method for calculating the distance CN by structure optimization calculation can be performed with reference to, for example, a method described on page 31-32 of “Chem3D v15 User Guide (perkinelmer.co.jp)” of PerkinElmer, Inc. More specifically, first, a structural formula is drawn by ChemDraw. Subsequently, structure optimization calculation is performed. At this time, for the sake of accuracy, it is preferable to perform structure optimization calculation by pcm calculation (designating chloroform as a solvent) under a condition of M062X/6-31+g(d) instead of MM2. In the structure optimization calculation, a distance between two fixed points (a carbon atom and a nitrogen atom) can be calculated by using Structure>Measurements>Display Distance Measurement on Chem3D.

In the compound represented by formula (1), when the divalent condensed ring group as X is a group represented by formula (X-1), formula (X-4), formula (X-16), formula (X-28), or formula (X-29), the distance CN is 11.0 to 17.0 Å. In the compound represented by formula (1), when the divalent condensed ring group as X is a group represented by formula (X-1), formula (X-4), or formula (X-28), the distance CN is 11.0 to 14.0 Å.

Examples of the compound represented by formula (1) include compounds represented by formulas (1)-1 to (1)-136.

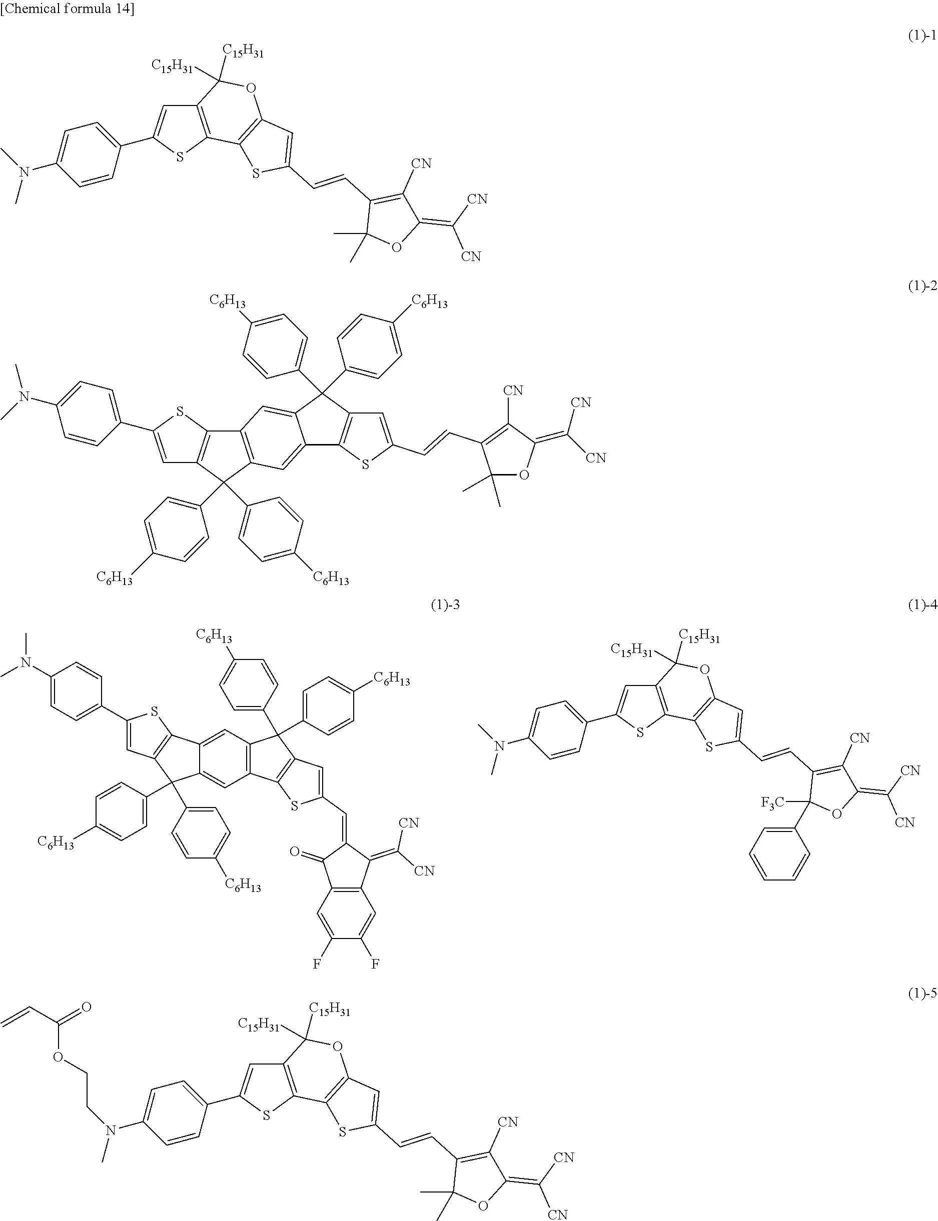

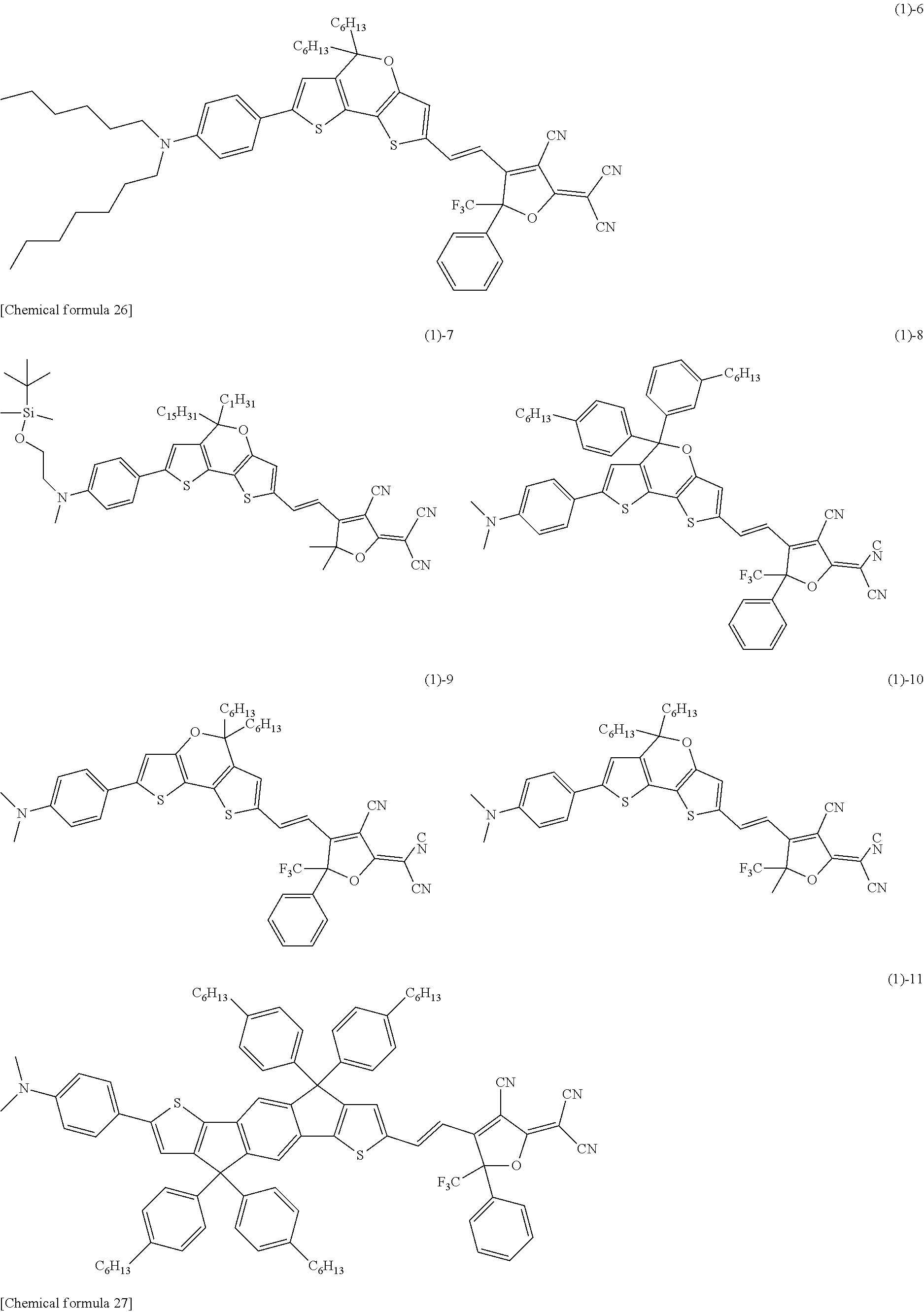

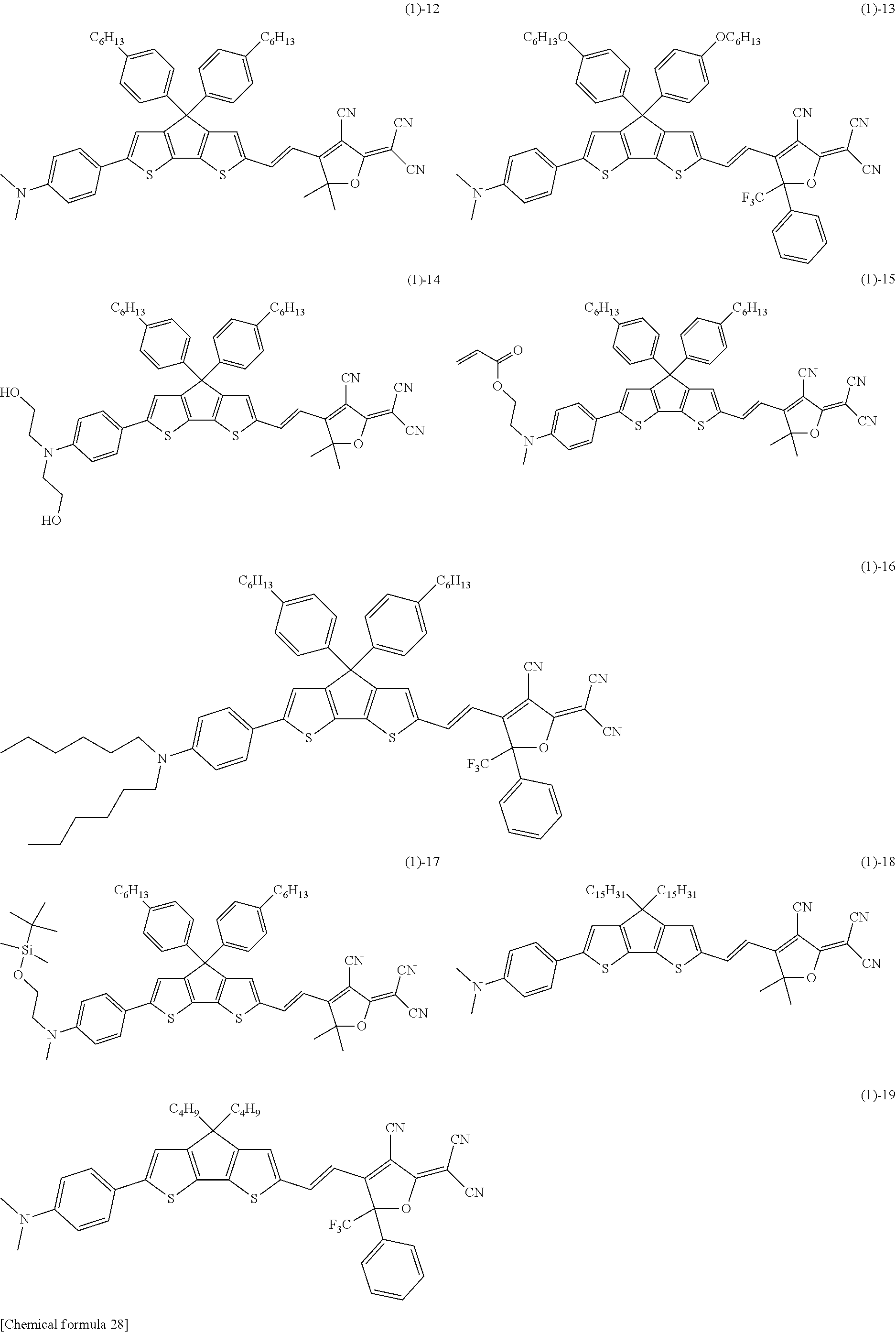

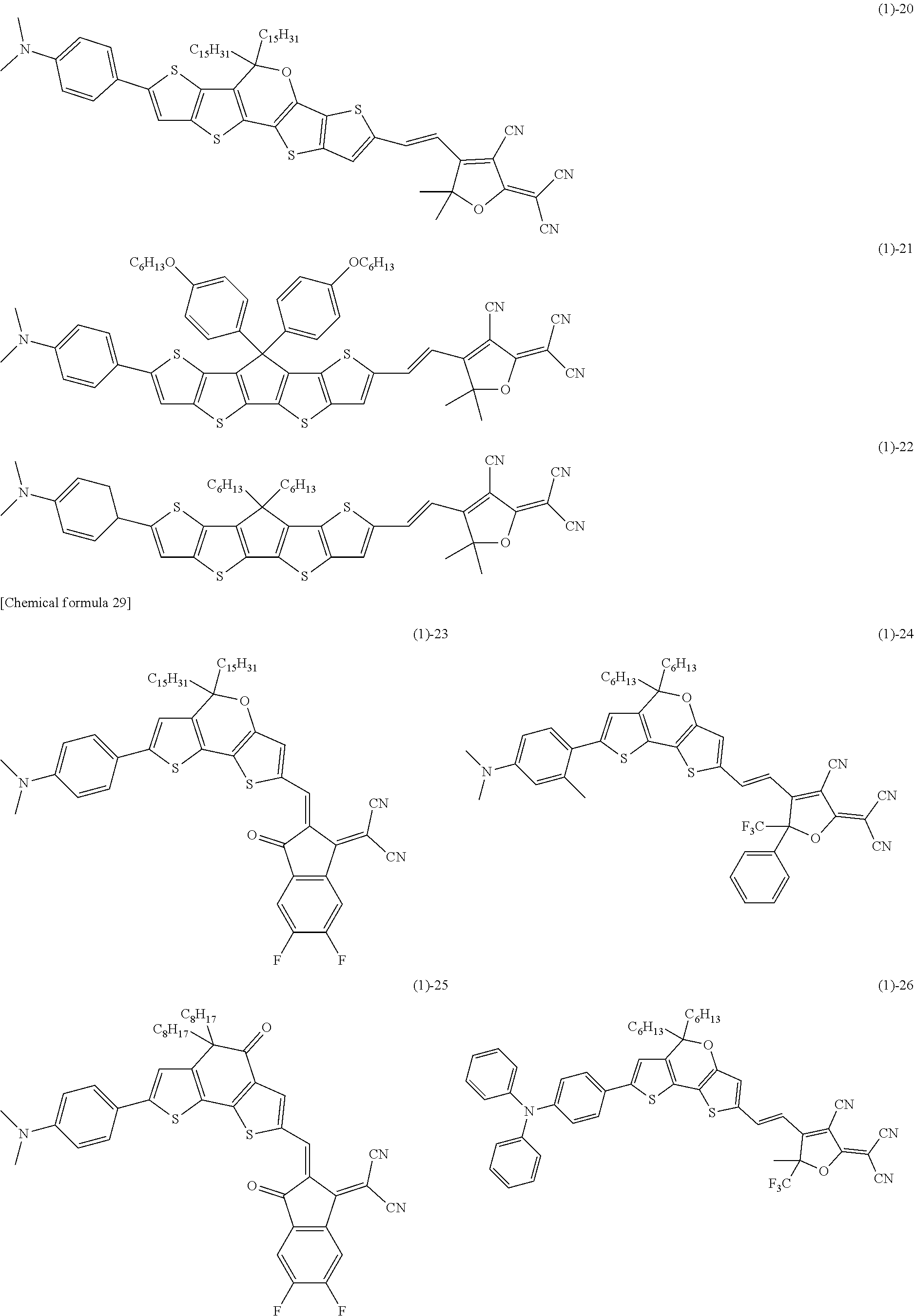

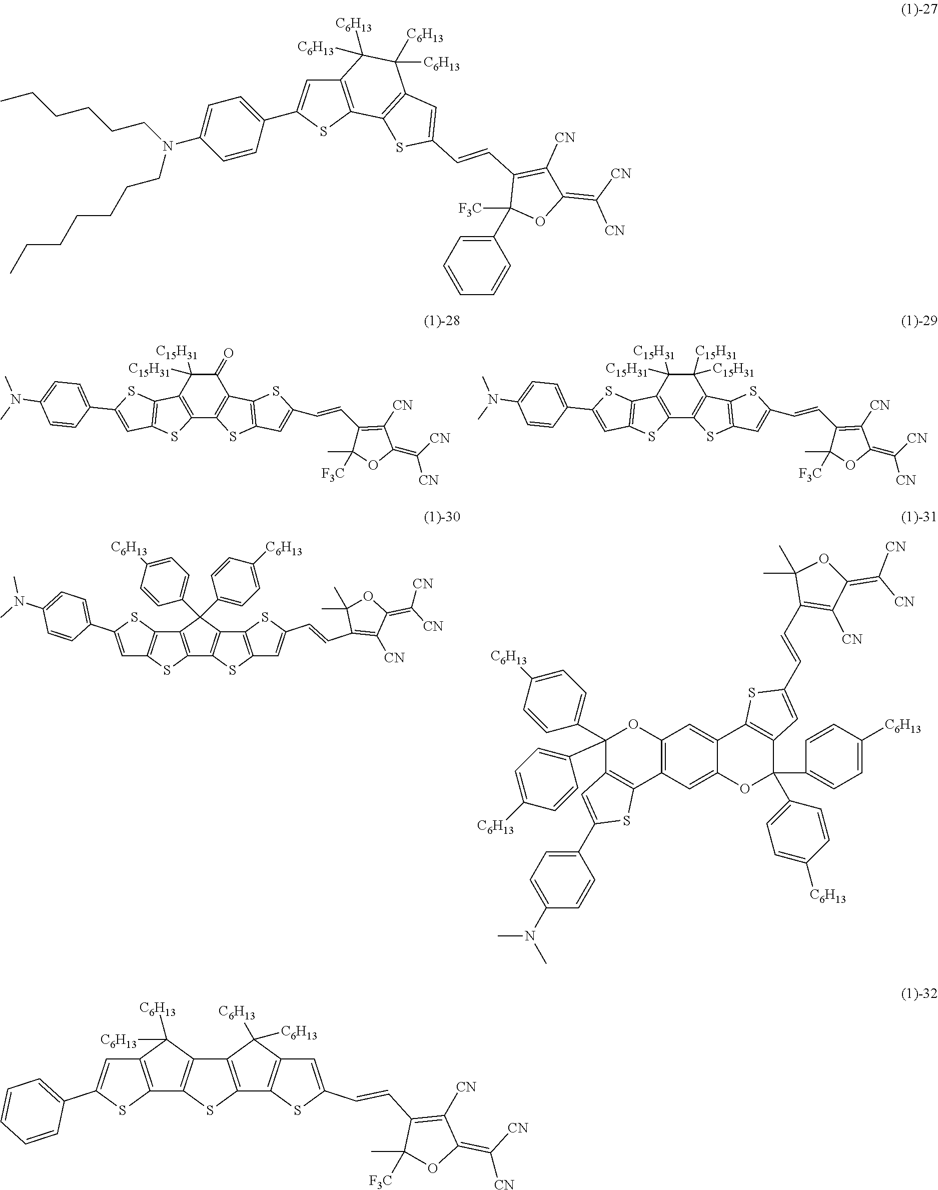

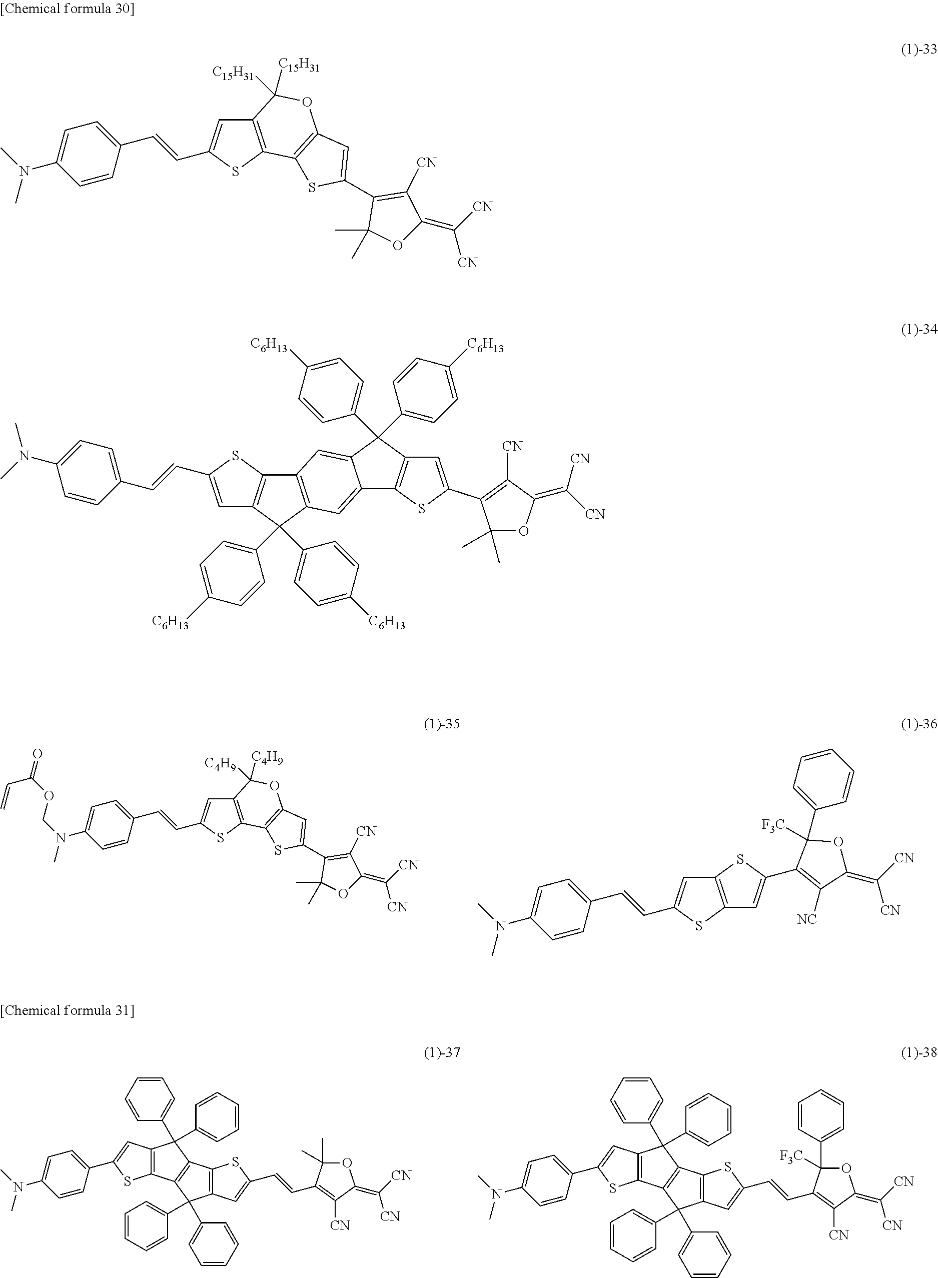

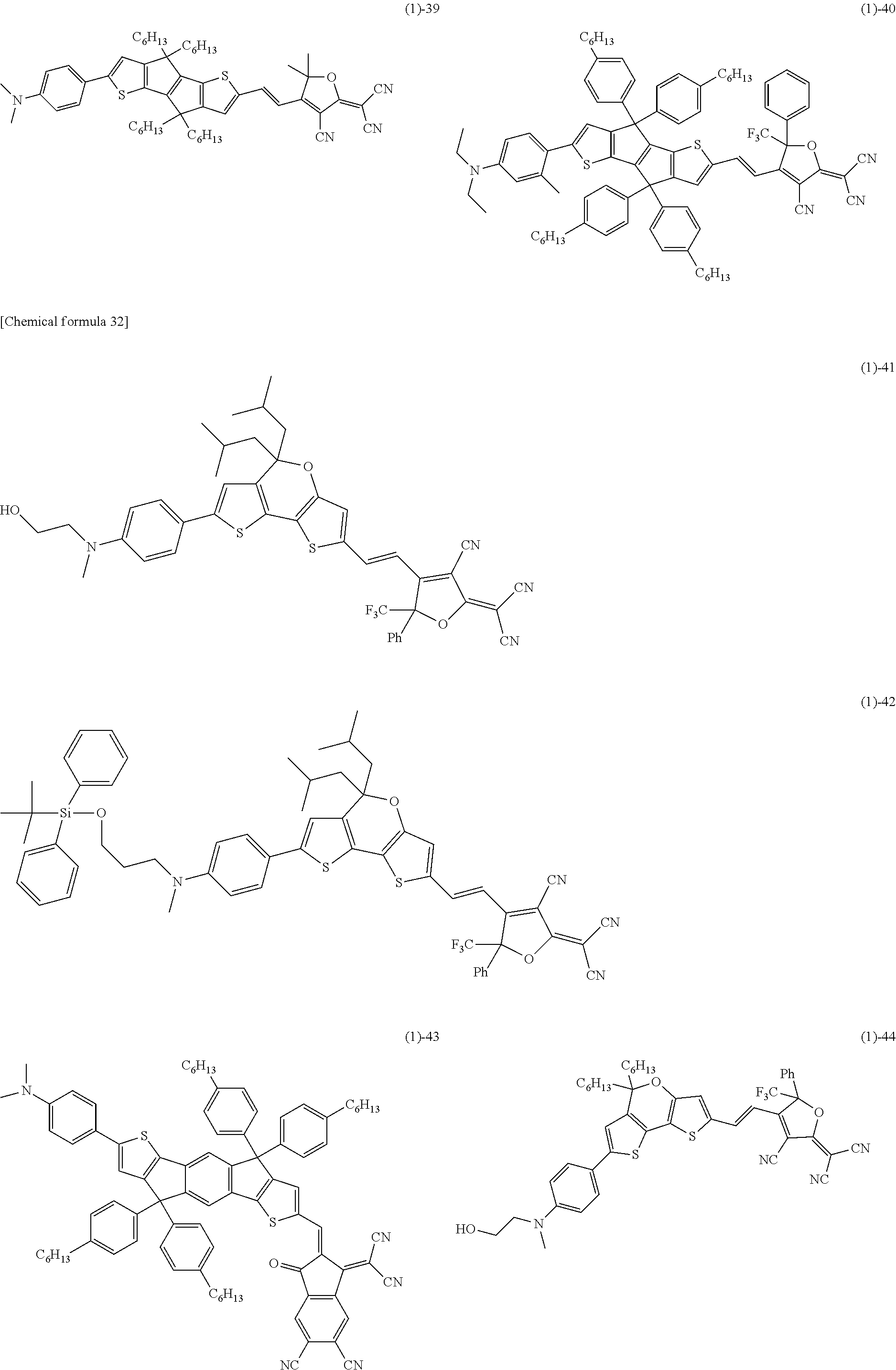

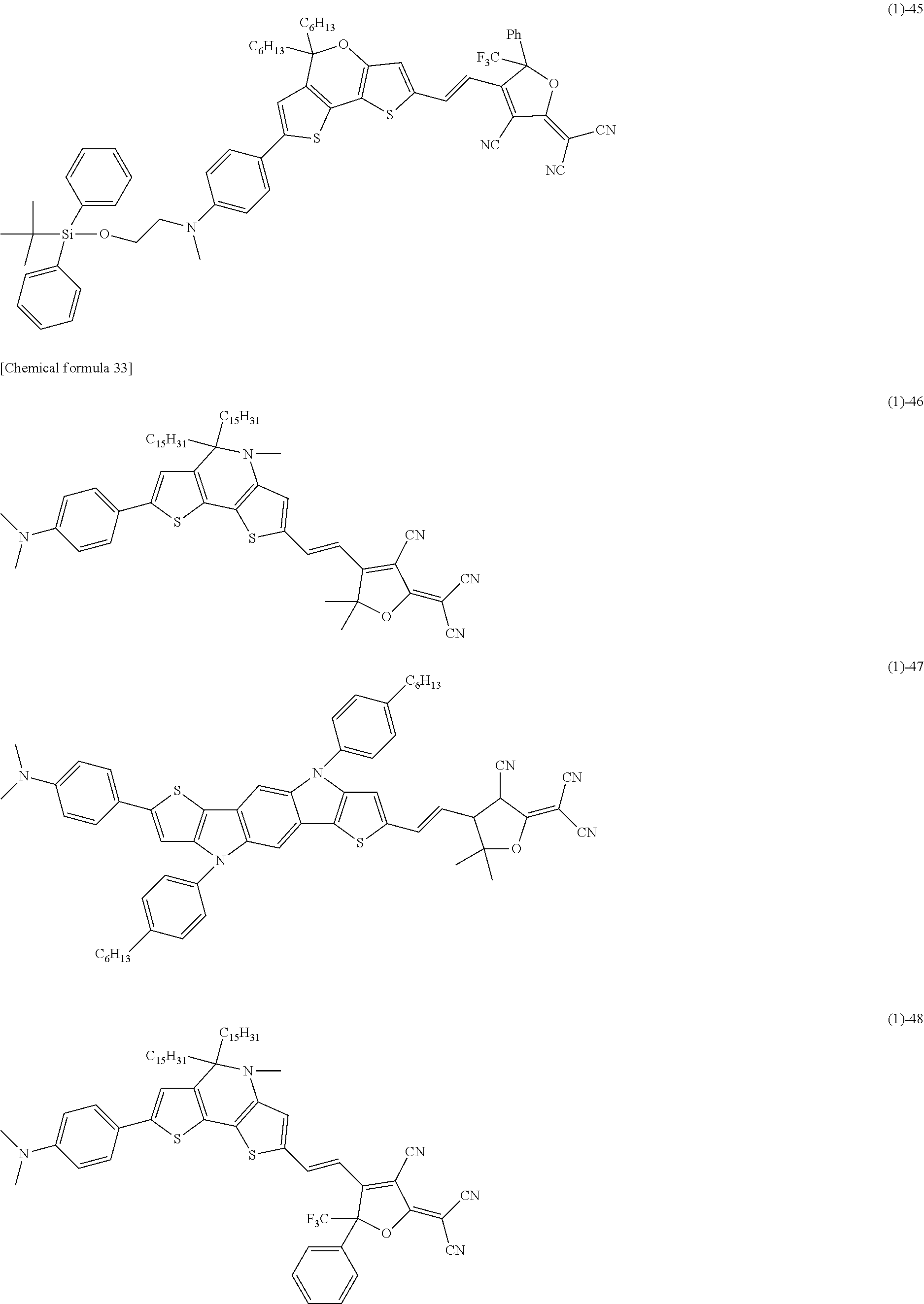

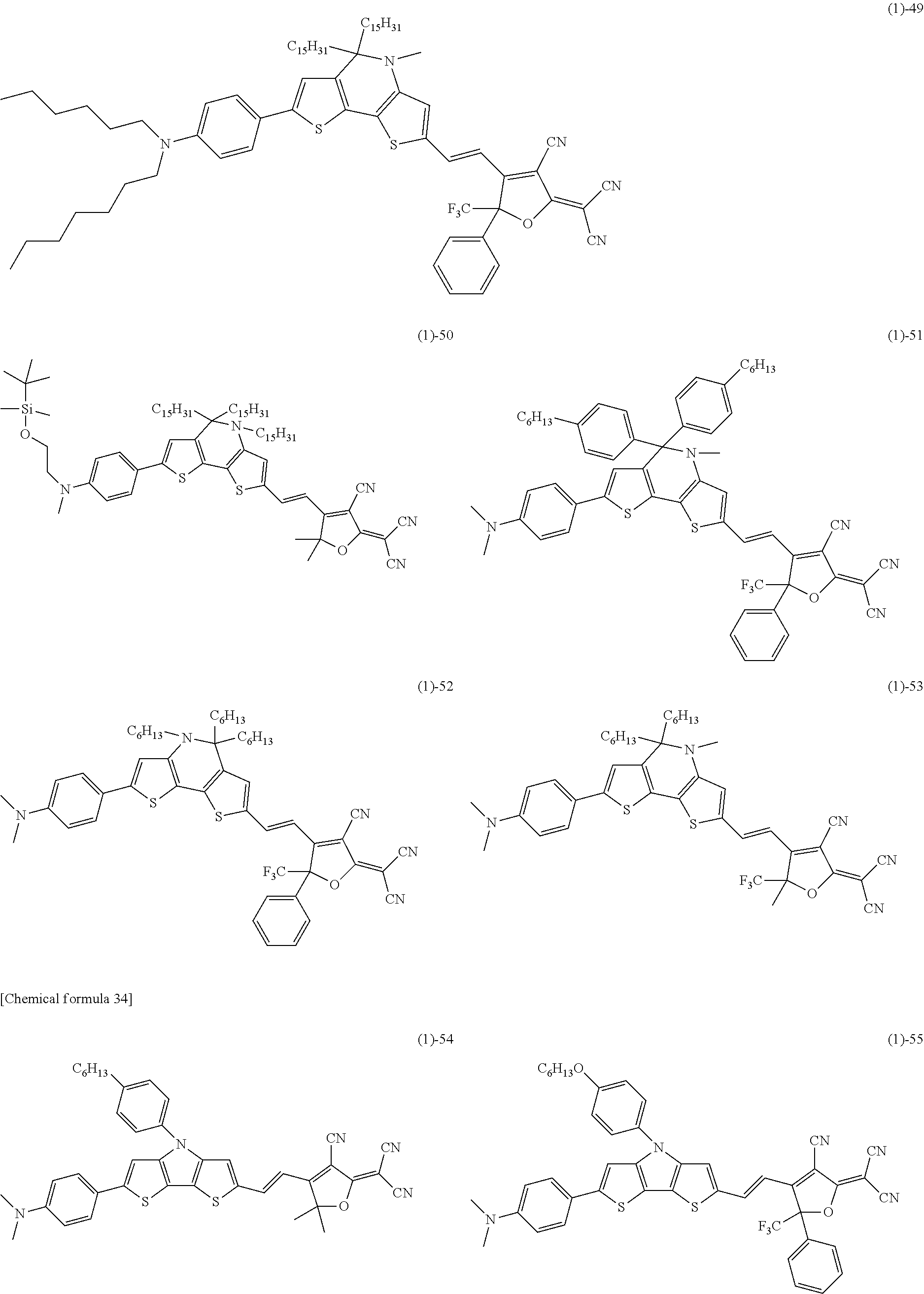

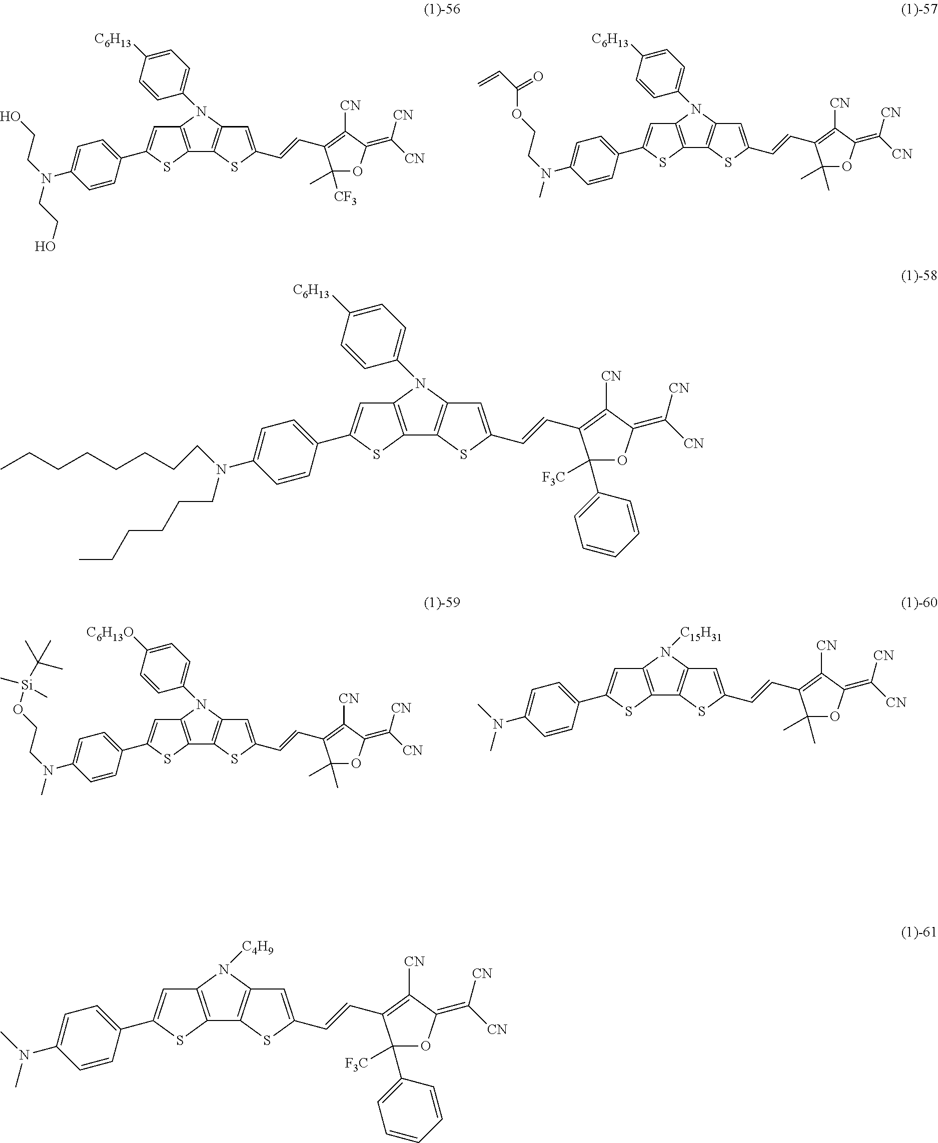

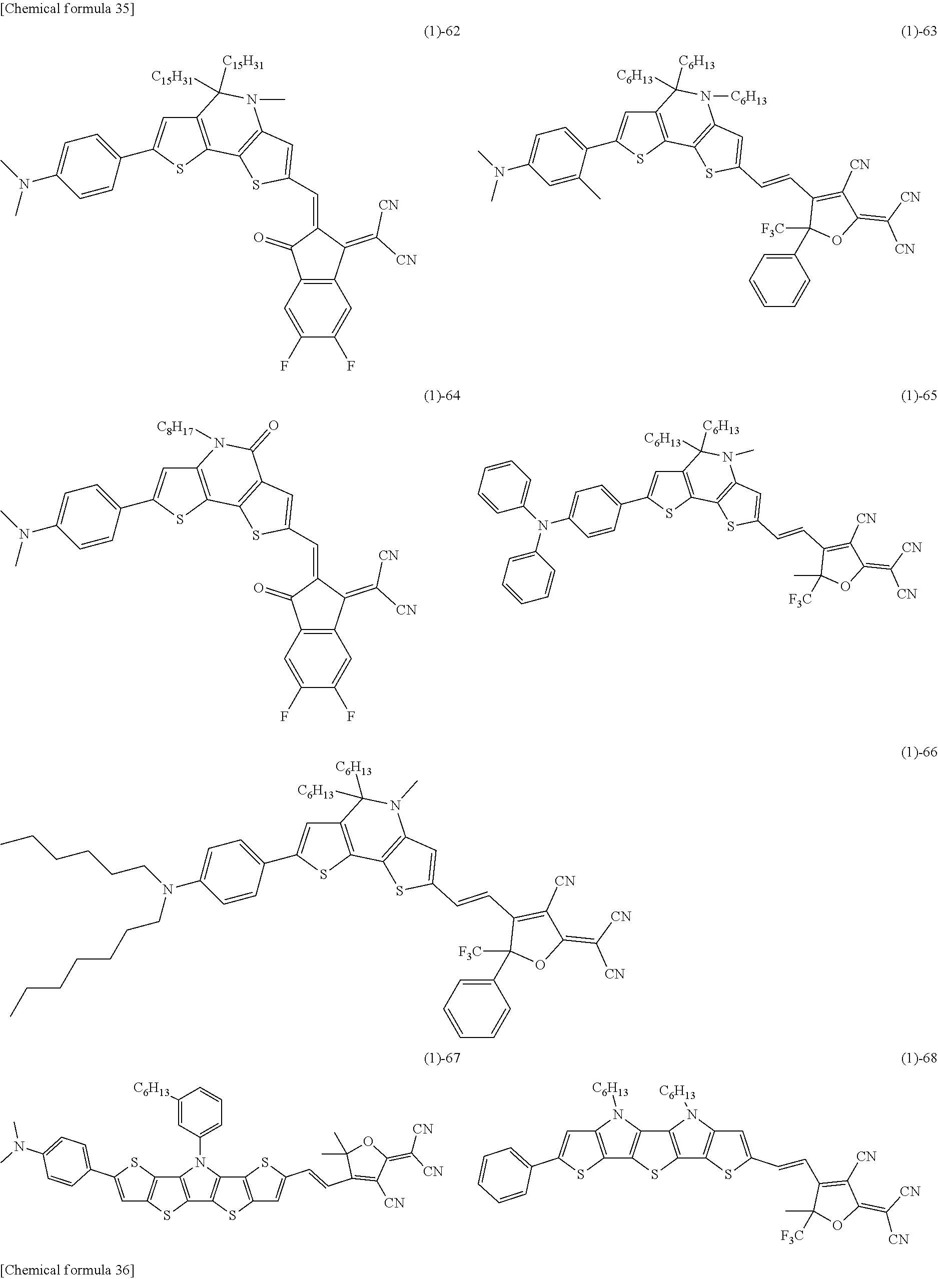

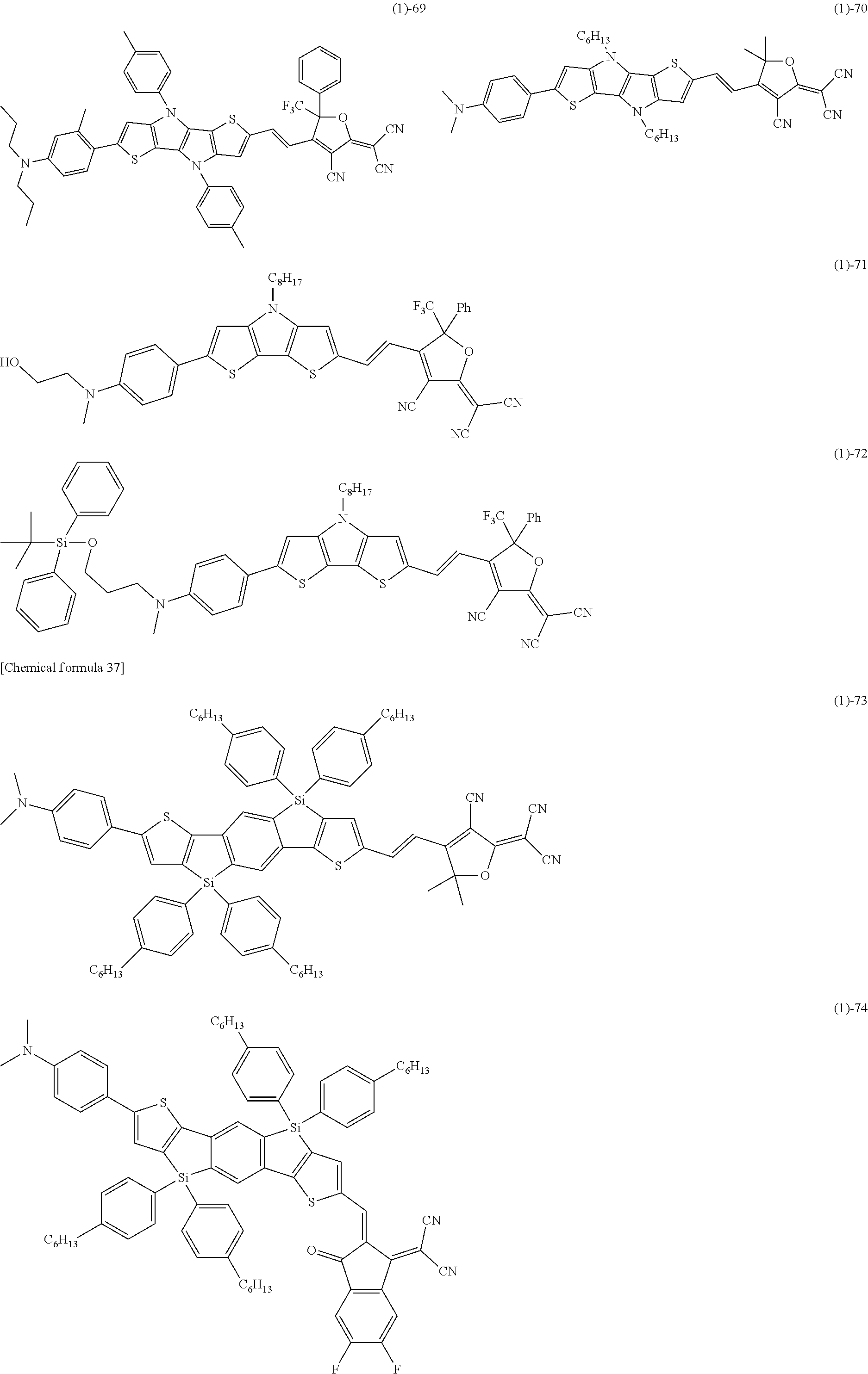

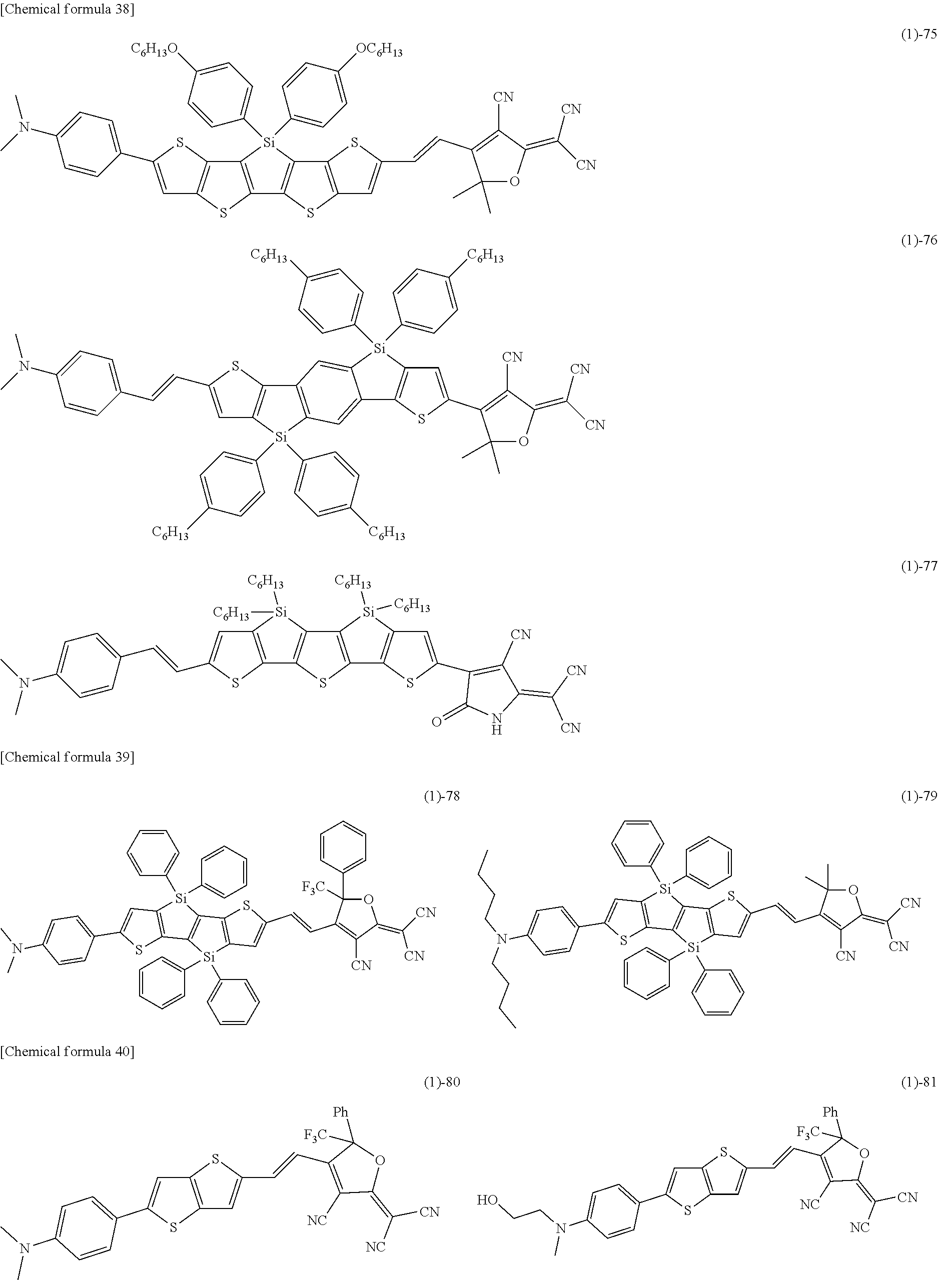

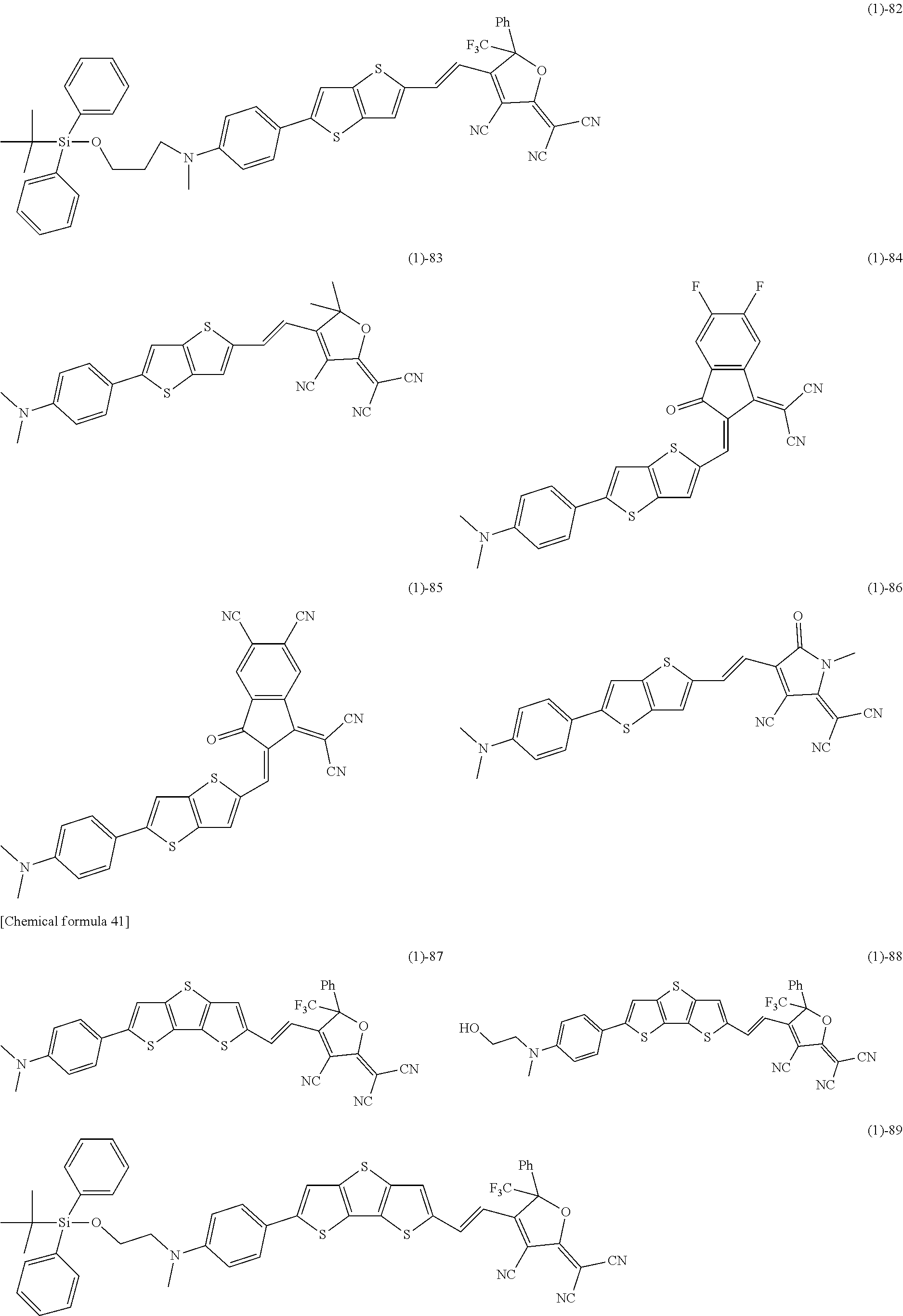

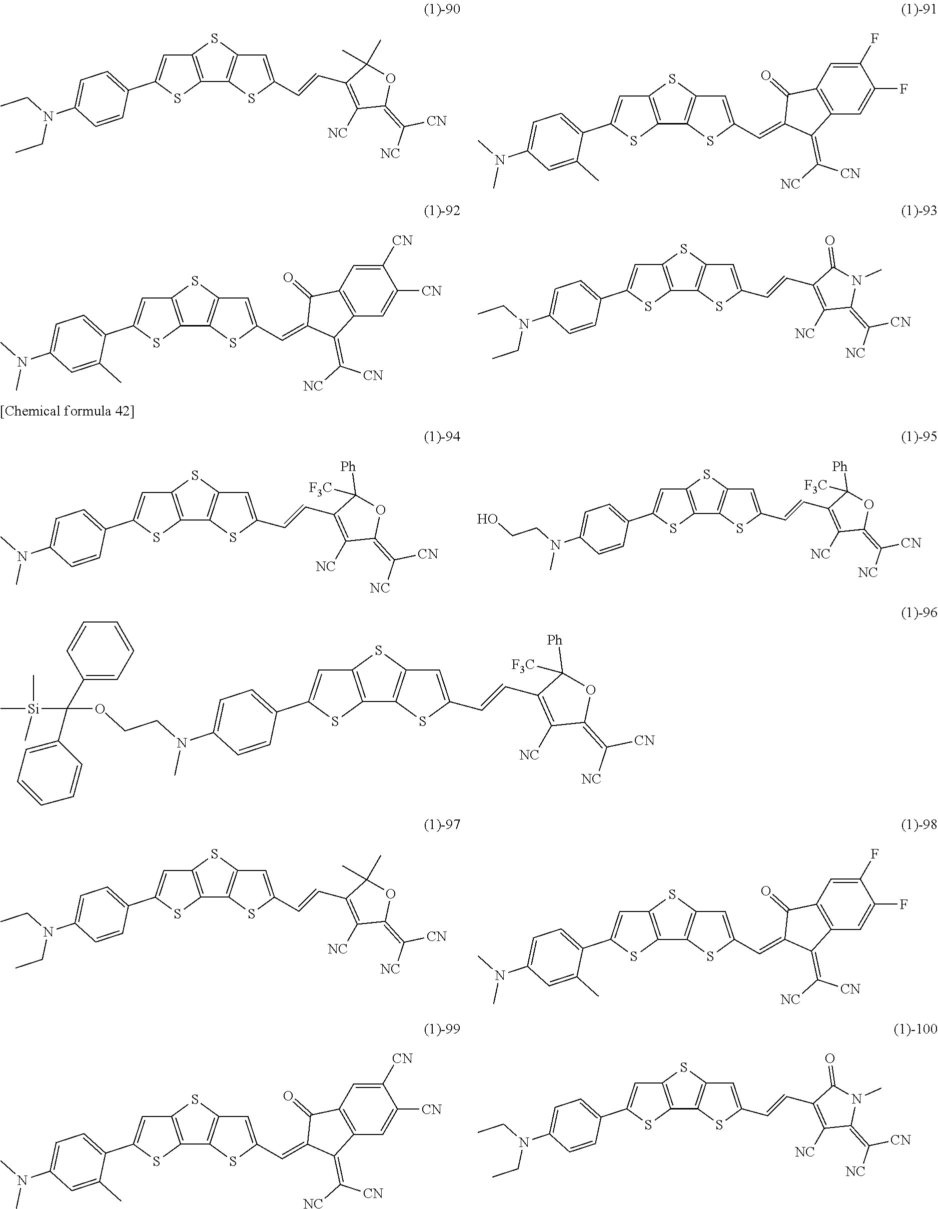

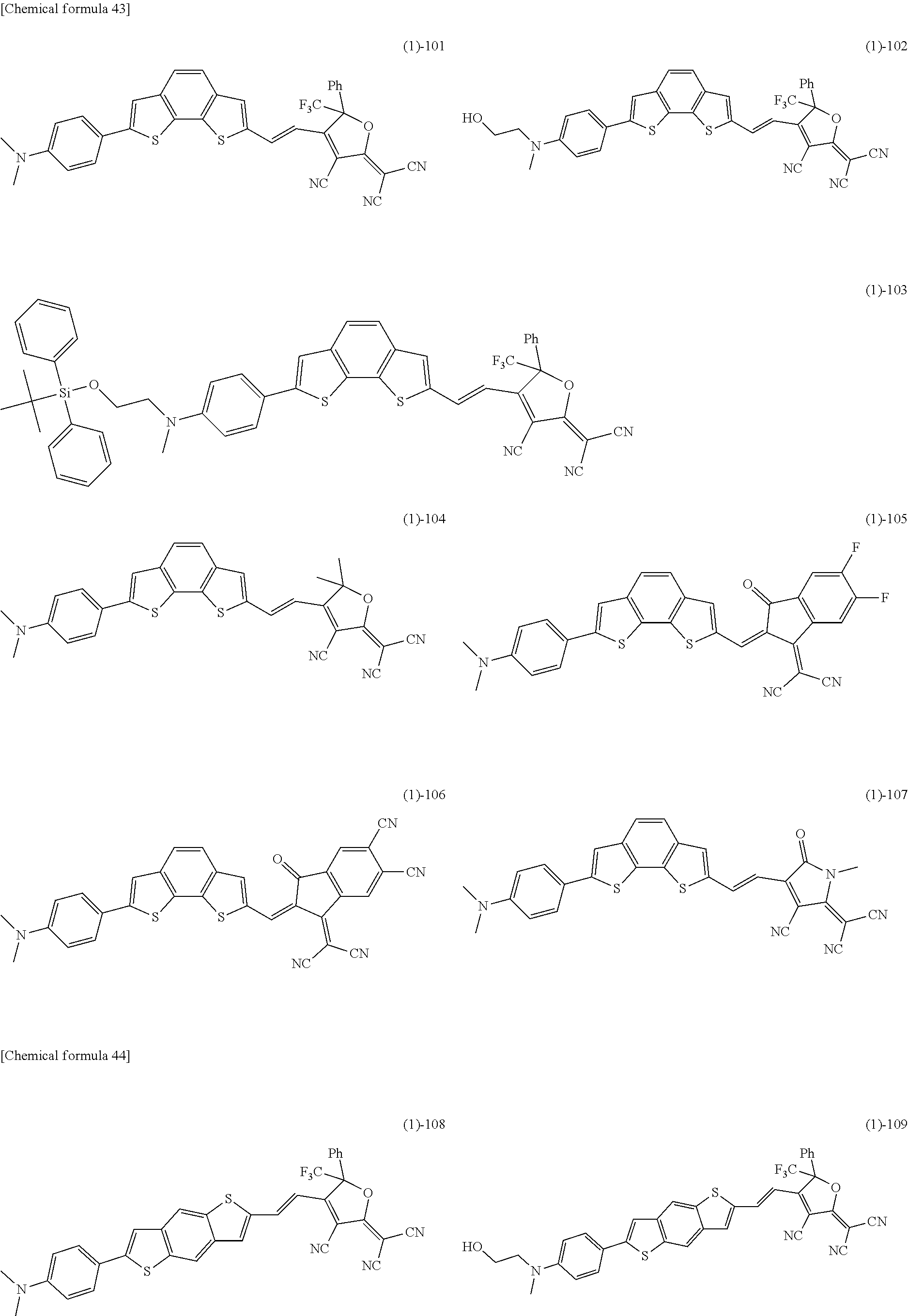

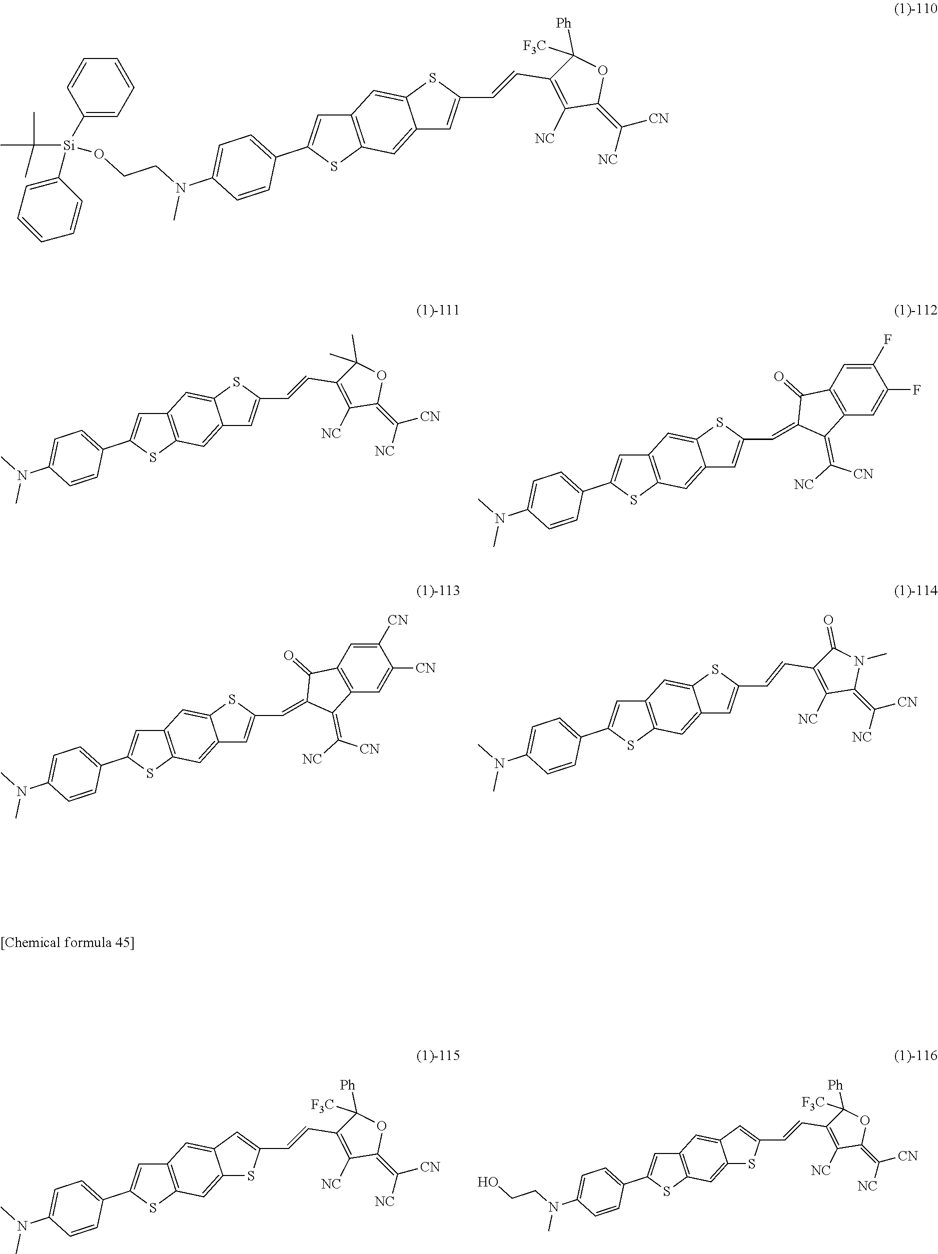

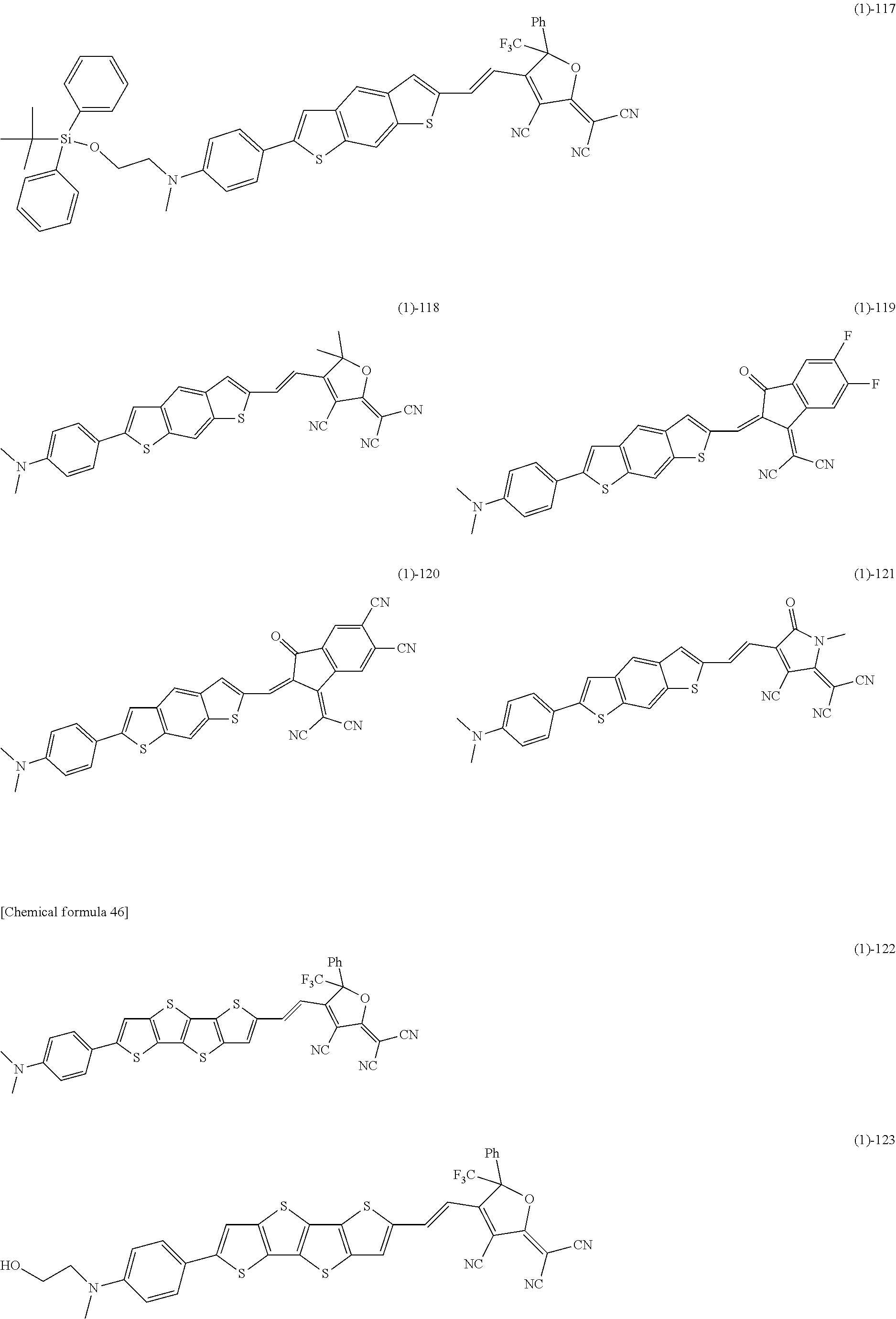

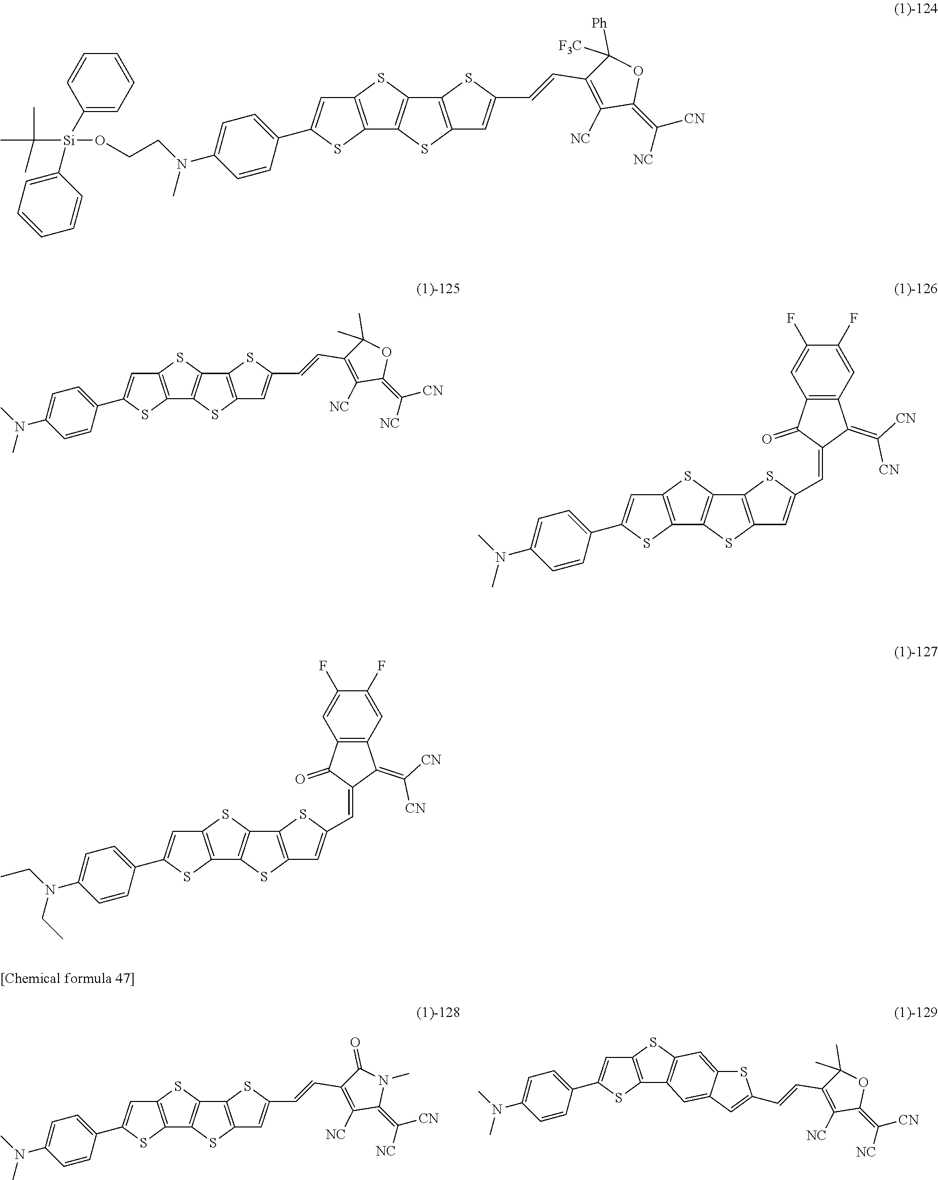

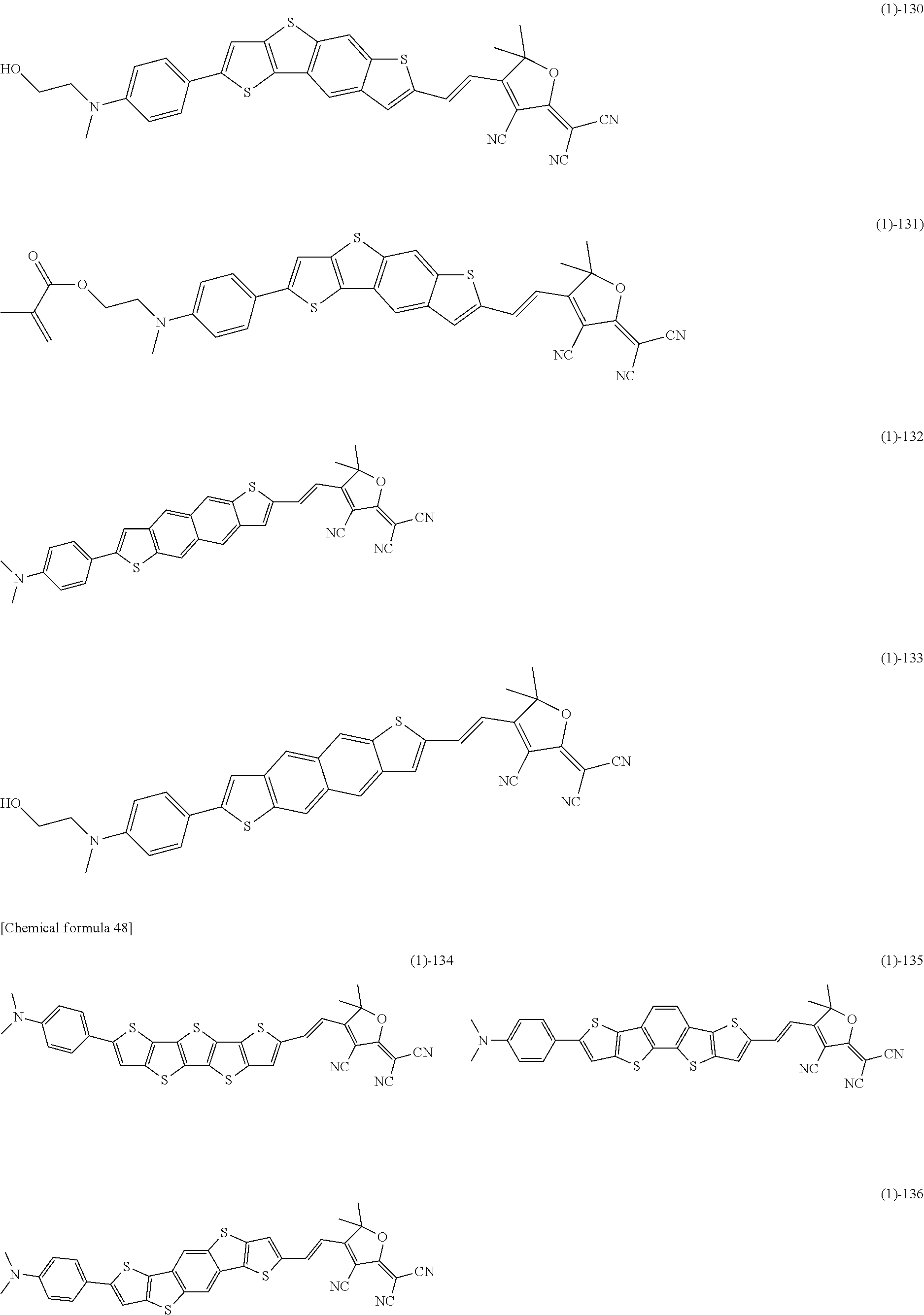

The compound represented by formula (1) is preferably a compound represented by any one of formulas (1)-1 to (1)-32, formulas (1)-37 to (1)-75, and formulas (1)-78 to (1)-136, more preferably a compound represented by any one of formula (1)-1, formulas (1)-4 to (1)-10, formulas (1)-12 to (1)-19, formulas (1)-23 to (1)-27, formula (1)-41, formula (1)-42, formulas (1)-44 to (1)-46, formulas (1)-48 to (1)-66, formula (1)-71, formula (1)-72, and formulas (1)-80 to (1)-121, most preferably a compound represented by any one of formula (1)-1, formulas (1)-4 to (1)-10, formulas (1)-12 to (1)-19, formula (1)-23, formula (1)-24, formula (1)-26, formula (1)-41, formula (1)-42, formula (1)-44, formula (1)-45, formulas (1)-54 to (1)-61, formula (1)-71, formula (1)-72, and formulas (1)-80 to (1)-100, and especially preferably a compound represented by any one of formula (1)-1, formulas (1)-4 to (1)-10, formulas (1)-12 to (1)-19, formula (1)-23, formula (1)-24, formula (1)-26, formula (1)-41, formula (1)-42, formula (1)-44, formula (1)-45, and formulas (1)-80 to (1)-86 because it is possible to suppress an optical loss of the EO film in the O band region.

When the compound represented by formula (1) is a compound represented by any one of formula (1)-1, formulas (1)-4 to (1)-10, formulas (1)-12 to (1)-19, formula (1)-23, formula (1)-24, formula (1)-26, formula (1)-41, formula (1)-42, formula (1)-44, formula (1)-45, formulas (1)-54 to (1)-61, formula (1)-71, formula (1)-72, and formulas (1)-80 to (1)-100, the distance CN is 11.0 to 17.0 Å. When the compound represented by formula (1) is a compound represented by any one of formula (1)-1, formulas (1)-4 to (1)-10, formulas (1)-12 to (1)-19, formula (1)-23, formula (1)-24, formula (1)-26, formula (1)-41, formula (1)-42, formula (1)-44, formula (1)-45, and formulas (1)-80 to (1)-86, the distance CN is 11.0 to 14.0 Å.

The compound of the present embodiment (compound represented by formula (1)) may have a cis-trans isomer. In the compound of the present embodiment, generation of a trans isomer tends to be dominant, but any one of a cis isomer, a trans isomer, and a cis-trans isomer mixture can be used. Among these, the compound of the present embodiment is preferably a trans isomer from a viewpoint of easily ensuring a polarizability.

### Method for Producing Compound

A method for producing the compound represented by formula (1) is not particularly limited. Here, the method for producing the compound represented by formula (1) will be described using a compound represented by formula (2b1-1) and a compound represented by formula (2a2) as examples.

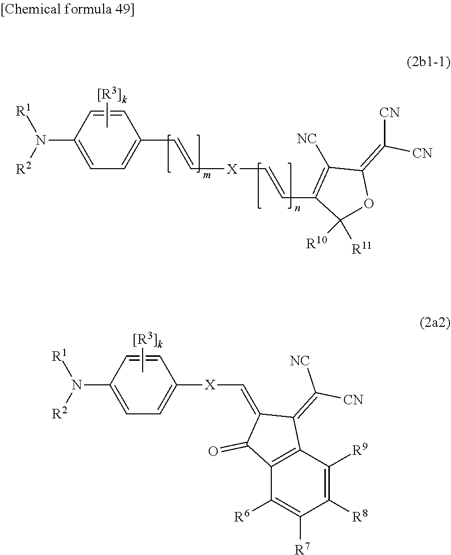

### Production Method A

A production method A is a method for producing a compound represented by formula (2b1-1) in which m is 0 and n is 1.

The production method A may include, for example, a step of preparing a compound (x-1) (polycyclic condensed cyclic compound), a step of formylating the compound (x-1) by a Vilsmeier reaction to obtain a compound (x-2), a step of brominating the compound (x-2) to obtain a compound (x-3), a step of coupling the compound (x-3) and a compound (x-4) by Suzuki coupling to obtain a compound (x-5), and a step of performing aldol condensation of the compound (x-5) and a compound (x-6) to obtain a compound represented by formula (3b1-1). By such a production method A, a compound represented by formula (2b1-1) in which m is 0 and n is 1 (compound represented by formula (2b1-1a)) can be obtained.

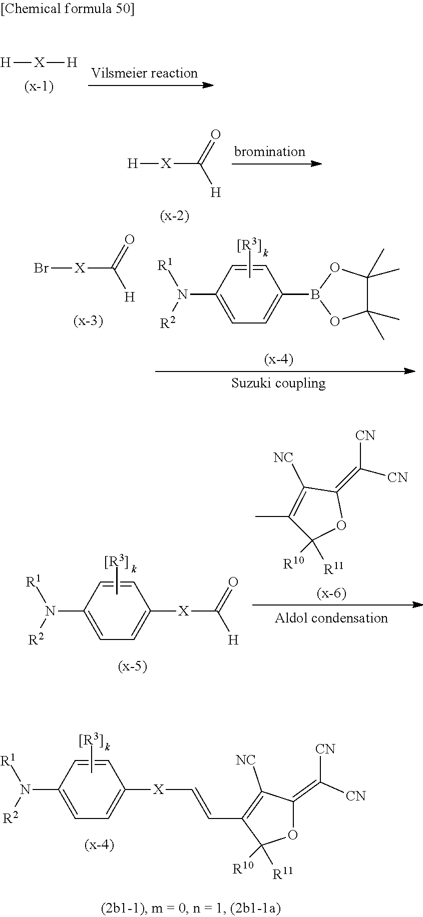

### Production Method B

A production method B is a method for producing a compound represented by formula (3b1-1) in which m is 1 and n is 0.

The production method B includes, for example, a step of preparing a compound (y-1) (polycyclic condensed cyclic compound), a step of formylating the compound (y-1) by a Vilsmeier reaction to obtain a compound (y-2), a step of brominating the compound (y-2) to obtain a compound (y-3), a step of subjecting the compound (y-3) and a compound (y-4) to a Wittig reaction (Horner-Wadsworth-Emmons reaction) to obtain a compound (y-5), a step of converting a bromo group of the compound (y-5) to an organometallic reactant with magnesium or the like, causing the organometallic reactant to react with a compound (y-6), and then performing hydrolysis to obtain a compound (y-7), and a step of subjecting the compound (y-7) and a compound (y-8) to Knoevenagel condensation to obtain a compound represented by formula (3b1-1). By such a production method B, the compound represented by formula (3b1-1) in which m is 1 and n is 0 can be obtained.

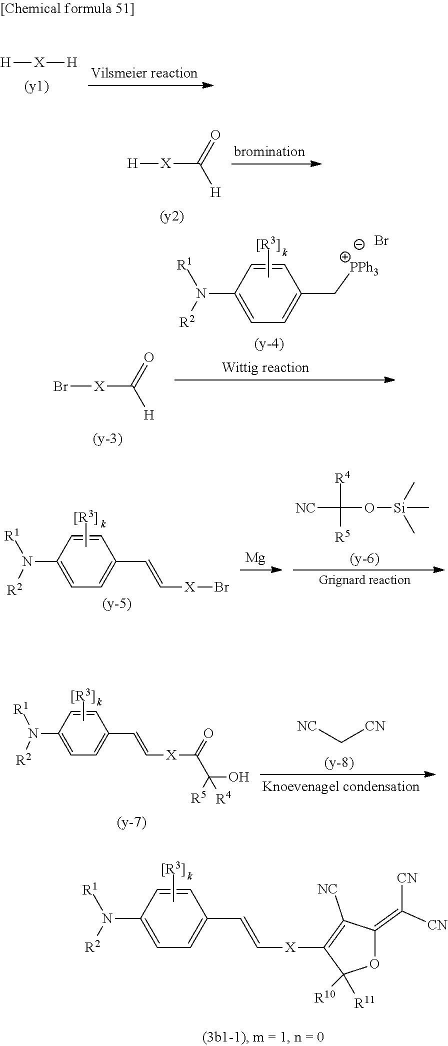

### Production Method C

A production method C is a method for producing a compound represented by formula (2a2).

The production method C may include, for example, a step of obtaining the compound (x-5) in a similar manner to the production method A, and a step of performing aldol condensation of the compound (x-5) and a compound (z-1) to obtain a compound represented by formula (2a2) By such a production method C, the compound represented by formula (2a2) can be obtained.

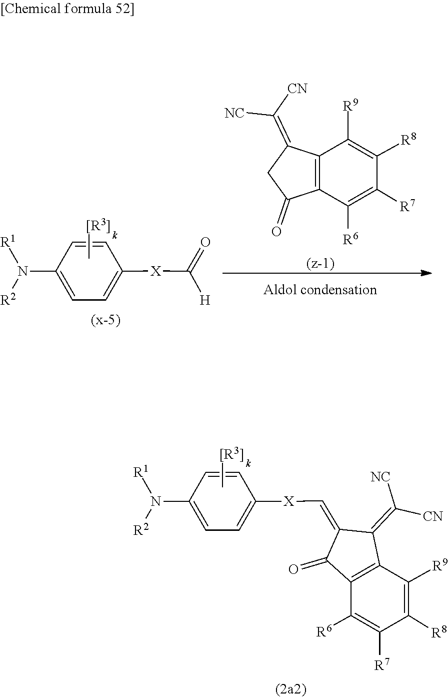

### Host Material

The EO film of the present embodiment preferably further contains a host material capable of dispersing the compound (A) or the compound represented by the formula (5). In order for the EO film to exhibit excellent EO characteristics, it is important that the compound (A) is uniformly dispersed at a high concentration in the host material. Therefore, the host material preferably exhibits high compatibility with the compound (A).

Examples of the host material include a resin such as a poly (meth)acrylate including polymethyl methacrylate (PMMA), polyimide, polycarbonate, polystyrene, polysulfone, polyether sulfone, a silicon-based resin, or an epoxy-based resin. These resins have excellent compatibility with the compound (A), and tend to have excellent transparency and moldability when being used as an EO element.

Examples of a method for dispersing the compound (A) in the host material include a method for dissolving the compound (A) and the host material in an organic solvent at an appropriate mixing ratio.

The host material may contain a resin having a reactive functional group capable of forming a covalent bond with the compound (A). Furthermore, at least a part of the compound (A) is preferably bonded to the resin having the reactive functional group. By inclusion of such a host material, the EO compound can be dispersed at a high density in the host material, and high EO characteristics can be achieved.

Examples of the reactive functional group include a haloalkyl group, an acyl halide group, an alkoxycarbonyl group, an aryloxycarbonyl group, a hydroxy group, an amino group, an isocyanate group, an epoxy group, and a carboxy group. The reactive functional group can form a covalent bond by reacting with, for example, a hydroxy group, an amino group, or an alkoxycarbonyl group in the compound (A).

The EO film, the EO element, and the like of the present embodiment can be produced by a known method (methods described in, for example, Oh et al., IEEE Journal of Selected Topics in Quantum Electronics, Vol. 7, No. 5, pp. 826-835, September/October 2001; Dalton et al., Journal of Materials Chemistry, 1999, 9, pp. 1905-1920; Toshikuni Kaino, Journal of the Institute of Electronics, Information and Communication Engineers, CVol. J84-C, No. 9, pp. 744-755, September 2001; and Ma et al., Advanced Materials, Vol. 14, No. 19, 2002, pp. 1339-1365).

The EO film of the present embodiment can be formed using an electro-optic composition (EO composition) containing, for example, the compound (A) or the compound represented by the formula (5), and containing a host material as necessary. The EO film can be obtained, for example, by a method including a step of applying an organic solvent solution of the EO composition onto a substrate by spin coating, and a step of heating and drying the obtained coating film.

The EO composition contains the compound (A) or the compound represented by the formula (5). The EO composition may further contain the host material. The EO composition can be suitably used for forming the EO film or forming the EO element. That is, the EO composition may be a composition for forming the EO film or a composition for forming the EO element.

The EO film may have a thickness of, for example, 0.01 to 100 μm.

The EO film has a maximum absorption wavelength (A max) of 760 to 830 nm. When λmax of the EO film is less than 760 nm, a dipole moment tends to decrease and an EO coefficient tends to decrease. When λmax of the EO film exceeds 830 nm, the wavelength of an entire absorption spectrum tends to be longer, and an optical loss of the EO film in the O band region tends to increase. The λmax of the EO film is preferably 770 nm or more, more preferably 780 nm or more, still more preferably 790 nm or more, and preferably 827 nm or less, more preferably 823 nm or less, still more preferably 820 nm or less.

A numerical value of λmax of the EO film can be increased, for example, by increasing an electron donating property of a donor and/or an electron withdrawing property of an acceptor, increasing the length of a n conjugated bridge, and the like.

The EO film has a long wavelength side half-value width of 90 to 120 nm. When the long wavelength side half-value width of the EO film is 90 nm or more, the compound (A) in the EO film tends to be easily dispersed uniformly. When the long wavelength side half-value width of the EO film is 120 nm or less, absorbance in the O band region can be reduced as much as possible, and an optical loss tends to be suppressed. The long wavelength side half-value width of the EO film is preferably 93 nm or more, more preferably 95 nm or more, still more preferably 100 nm or more, and preferably 119 nm or less, more preferably 118 nm or less, still more preferably 117 nm or less.

A numerical value of the long wavelength side half-value width of the EO film can be increased, for example, by increasing an electron donating property of a donor and/or an electron withdrawing property of an acceptor, increasing the length of a n conjugated bridge, and the like.

A wavelength of the EO film, such as λmax can be measured using a spectrophotometer. A measurement method can be performed, for example, as follows. First, a coating solution is prepared by adjusting a mass ratio of the compound (A) and a host material (for example, polymethyl methacrylate (PMMA)) to be 2:8, and dissolving the compound (A) and the host material in an organic solvent (for example, o-dichlorobenzene or chlorobenzene). Next, the coating solution is applied to a substrate (ITO-coated glass, quartz glass, or the like) under a condition of 500 to 3000 rpm using a spin coater, and vacuum-dried for one hour around a glass transition temperature (Tg) to prepare an EO film having a predetermined film thickness. A spectral spectrum (UV-visible spectrum) of the EO film thus prepared is measured using a spectrophotometer.

In this manner, a wavelength of the EO film, such as λmax can be obtained.

### Electro-Optic Element

The EO element of the present embodiment includes the above EO film. As described above, since an optical loss in the O band region can be suppressed, the EO element of the present embodiment can also be suitably used for optical communication in the O band region.

An application of the EO element of the present embodiment is not limited to an optical modulator as long as the EO element has the above EO film. The EO element of the present embodiment can be used for, in addition to the optical modulator (application for ultra-high-speed, application for optical interconnect, application for optical signal processing, and the like), for example, an optical switch, an optical memory, a wavelength converter, an electric field sensor using a microwave, a millimeter wave, a terahertz wave, or the like, a biological potential sensor using a myoelectric potential, an electroencephalogram, or the like, an optical spatial modulator, an optical scanner, and the like, and can be further used for signal transmission by light between electronic circuits by combination with an electronic circuit, and the like.

### EXAMPLES

Hereinafter, the present invention will be described in more detail with reference to Examples, but the present invention is not limited to these Examples.

### 1. Synthesis of Compound, Calculation of Dipole Moment, and Calculation of Distance CN

### Example 1-1

### (A) Synthesis of Compound (1)

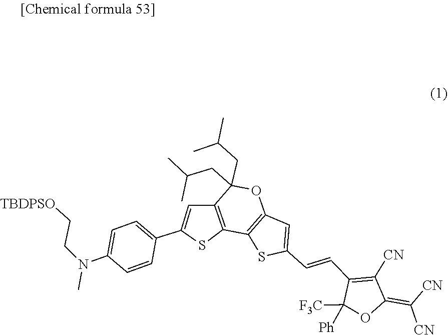

Synthesis of compound (1-b)

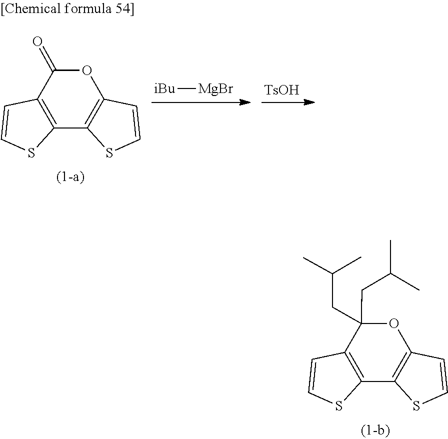

Into a 3 L eggplant flask equipped with a three-way cock and a gas introduction tube, 100 g (480 mmol) of a compound (1-a) synthesized by a method described in WO 2011/052709 A and 800 mL of dehydrated tetrahydrofuran (manufactured by KANTO CHEMICAL CO., INC.) were put. A stirrer was further put thereinto, the inside was replaced with nitrogen, and the solution was cooled to 0° C. 1056 mL (1056 mmol) of a 1 M isobutylmagnesium bromide tetrahydrofuran solution (manufactured by Tokyo Chemical Industry Co., Ltd.) was added dropwise thereto, and then the resulting mixture was stirred for 24 hours to be caused to react. After completion of the reaction, 1000 mL of deionized water and 2500 mL of toluene were added thereto, and the organic layer was washed with deionized water. After washing, the organic layer was dried over anhydrous magnesium sulfate. Thereafter, the insoluble matter was separated by filtration, and the filtrate was concentrated by a rotary evaporator to obtain a reaction product as a brown oily matter. The obtained amount was 144 g.

Subsequently, into a 3 L eggplant flask equipped with a three-way cock at a top and a gas introduction tube, the reaction product, 21 g (111 mmol) of p-toluenesulfonic acid monohydrate (manufactured by Tokyo Chemical Industry Co., Ltd.), and 1440 mL of dehydrated toluene (manufactured by KANTO CHEMICAL CO., INC.) were put. A stirrer was further put thereinto, and the inside was replaced with nitrogen. Reaction was caused under reflux conditions for one hour while the reaction mixture was stirred with a magnetic stirrer. The obtained reaction mixture was washed with deionized water. After washing, the organic layer was dried over anhydrous magnesium sulfate. Thereafter, the insoluble matter was separated by filtration, and the filtrate was concentrated by a rotary evaporator. The concentrate was purified with a silica gel column (hexane) to obtain a compound (1-b) as a pale orange oily matter. The obtained amount was 74 g (yield: 43%). A measurement result of an ¹H-NMR spectrum of the compound (1-b) is as follows.

¹H-NMR (400 MHz, CDCl₃): δ (ppm)=7.10 (d, 1H), 6.95 (d, 1H), 6.68 (d, 1H), 6.64 (d, 1H), 1.90-1.72 (m, 6H), 0.87 (d, 6H), 0.84 (d, 6H).

### Synthesis of Compound (1-c)

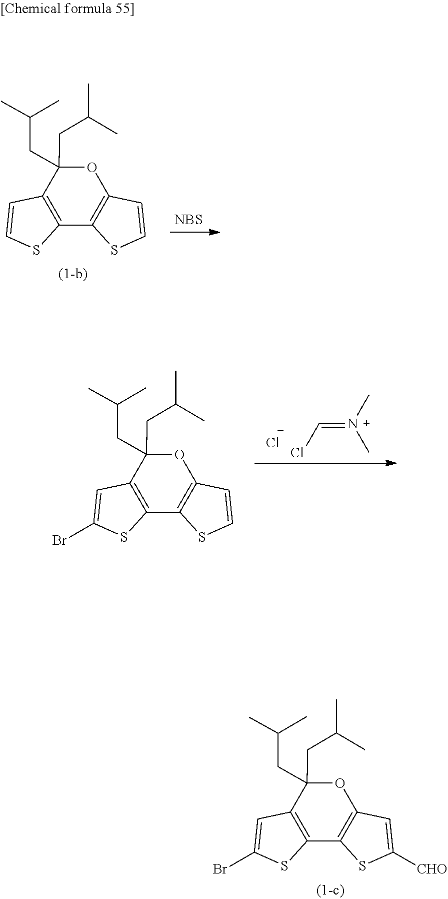

Into a 2 L eggplant flask equipped with a three-way cock at a top and a gas introduction tube, 38.7 g (126 mmol) of the synthesized compound (1-b) and 387 mL of dehydrated tetrahydrofuran (manufactured by FUJIFILM Wako Pure Chemical Corporation) were put. A stirrer was further put thereinto, the inside was replaced with nitrogen, and the solution was cooled to −40° C. 22.7 g (128 mmol) of N-bromosuccinimide (manufactured by Tokyo Chemical Industry Co., Ltd.) was added thereto, and then the resulting mixture was stirred at −40° C. for two hours to be caused to react. The temperature of the reaction mixture was raised to room temperature (25° C.). Thereafter, 97.1 g (758 mmol) of (chloromethylene) dimethyliminium chloride (manufactured by Tokyo Chemical Industry Co., Ltd.) and 487 mL of dehydrated dimethylformamide were added thereto, and the resulting mixture was caused to react at 60° C. for five hours while being stirred with a magnetic stirrer. 731 mL of toluene was added to the reaction mixture, and the organic layer was washed with deionized water. After washing, the organic layer was dried over anhydrous magnesium sulfate. Thereafter, the insoluble matter was separated by filtration, and the filtrate was concentrated by a rotary evaporator to obtain a crude product. The obtained crude product was purified with a silica gel column (hexane/ethyl acetate=80/20) to obtain a compound (1-c) as a green solid. The obtained amount was 36.4 g (yield: 70%). A measurement result of an ¹H-NMR spectrum of the compound (1-c) is as follows.

¹H-NMR (400 MHz, CDCl₃): δ (ppm)=9.74 (s, 1H), 7.21 (s, 1H), 6.69 (s, 1H), 1.86-1.71 (m, 6H), 0.87 (d, 6H), 0.85 (d, 6H).

### Synthesis of Compound (1A-b)

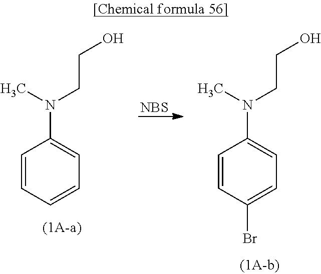

Into a 1 L eggplant flask equipped with a gas introduction tube and an induction stirring type stirrer, 55.47 g (366.8 mmol) of 2-(methylphenylamino) ethanol (compound (1A-a), manufactured by Tokyo Chemical Industry Co., Ltd.) and 832 mL of dehydrated dimethylformamide (manufactured by FUJIFILM Wako Pure Chemical Corporation) were put. The inside was replaced with nitrogen, and the solution was cooled to −40° C. 65.29 g (366.8 mmol) of N-bromosuccinimide (manufactured by Tokyo Chemical Industry Co., Ltd.) was added thereto. Thereafter, the temperature was raised to −15° C., and reaction was caused for two hours. The temperature of the reaction mixture was raised to room temperature (25° C.), then 455 g of a 10% sodium sulfite aqueous solution and 1110 mL of toluene were added thereto, and the organic layer was washed with deionized water. After washing, the organic layer was dried over anhydrous magnesium sulfate. Thereafter, the insoluble matter was separated by filtration, and the filtrate was concentrated and dried by a rotary evaporator to obtain a compound (1A-b) as a colorless oil. The obtained amount was 72.05 g (yield: 85%).

A measurement result of an ¹H-NMR spectrum of the compound (1A-b) is as follows.

¹H-NMR (400 MHz, CDCl₃): δ(ppm)=7.27 (d, 2H), 6.63 (d, 2H), 3.80-3.74 (m, 2H), 3.42 (t, 2H), 2.92 (3H), 1.90 (t, 1H).

### Synthesis of Compound (1A-c)

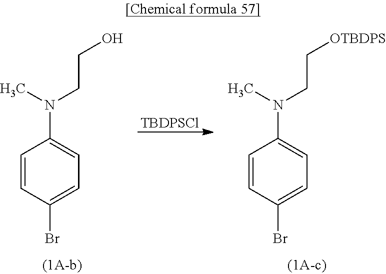

Into a 1 L eggplant flask equipped with a gas introduction tube and an induction stirring type stirrer, 71.93 g (312.6 mmol) of the compound (1A-b), 42.56 g (625.2 mmol) of imidazole (manufactured by Tokyo Chemical Industry Co., Ltd.), and 832 mL of dehydrated tetrahydrofuran (manufactured by FUJIFILM Wako Pure Chemical Corporation) were put. The inside was replaced with nitrogen, and the solution was cooled to 0° C. 88.51 g (322.0 mmol) of tert-butyldiphenylchlorosilane (TBDPSCl, manufactured by Tokyo Chemical Industry Co., Ltd., hereinafter, a tert-butyldiphenylsilyl group is also referred to as “TBDPS”) was added thereto. Thereafter, the temperature was raised to room temperature (25° C.), and reaction was caused for three hours. 2929 mL of hexane was added to the reaction mixture, and the organic layer was washed with deionized water. After washing, the organic layer was dried over anhydrous magnesium sulfate. Thereafter, the insoluble matter was separated by filtration, and the filtrate was concentrated and dried by a rotary evaporator to obtain a compound (1A-c) as a colorless oil. The obtained amount was 145.5 g (yield: 99%).

### Synthesis of Compound (1A-d)

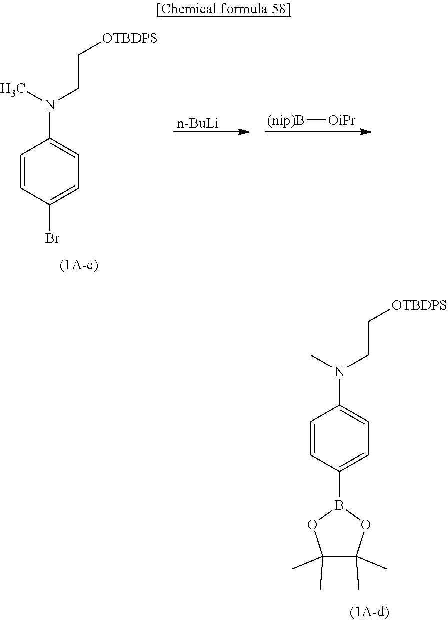

Into a 3 L eggplant flask equipped with a three-way cock at a top and a gas introduction tube, 145.6 g (310.8 mmol) of the compound (1A-c) and 1456 mL of dehydrated tetrahydrofuran (manufactured by FUJIFILM Wako Pure Chemical Corporation) were put. The inside was replaced with nitrogen, and the solution was cooled to −65° C. 239 mL (372.9 mmol) of a 1.6 M n-butyl lithium tetrahydrofuran solution (manufactured by KANTO CHEMICAL CO., INC.) was added dropwise thereto, and then the resulting mixture was stirred for one hour to be caused to react. 75.17 g (404.0 mmol) of 2-isopropoxy-4,4,5,5-tetramethyl-1,3,2-dioxaborolane (manufactured by Tokyo Chemical Industry Co., Ltd.) was added thereto, and the temperature was raised to room temperature (25° C.) over two hours. 2884 mL of hexane was added to the reaction mixture, and the organic layer was washed with deionized water. After washing, the organic layer was dried over anhydrous magnesium sulfate. Thereafter, the insoluble matter was separated by filtration, and the filtrate was concentrated by a rotary evaporator to obtain a crude product. The crude product was crystallized twice using acetonitrile, and then the solid was dried under reduced pressure to obtain a compound (1A-d) as a white solid. The obtained amount was 124.1 g (yield: 77%).

A measurement result of an 1H-NMR spectrum of the compound (1A-d) is as follows.

¹H-NMR (400 MHz, CDCl₃): δ (ppm)=7.64-7.57 (m, 6H), 7.42-7.31 (m, 6H), 6.54 (d, 2H), 3.79 (t, 2H), 3.49 (t, 2H), 2.95 (s, 3H), 1.31 (s, 12H), 1.02 (s, 9H).

### Synthesis of Compound (1-d)

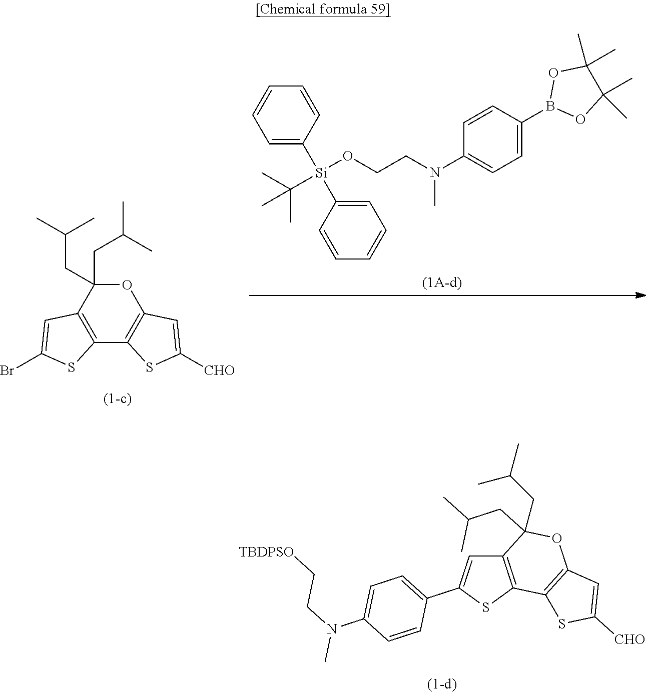

Into a 300 mL four-necked flask equipped with a Dimroth with a three-way cock at a top and a gas introduction tube, 6.00 g (14.5 mmol) of the synthesized compound (1-c), 8.23 g (16.0 mmol) of the synthesized compound (1A-d), and 90 mL of dehydrated tetrahydrofuran (manufactured by FUJIFILM Wako Pure Chemical Corporation) were put. A stirrer was further put thereinto, and the inside was replaced with nitrogen. 0.42 g (0.36 mmol) of tris(dibenzylideneacetone) dipalladium (0) (manufactured by N.E. CHEMCAT CORPORATION), 0.44 g (1.45 mmol) of tri-tert-butylphosphonium tetrafluoroborate (manufactured by FUJIFILM Wako Pure Chemical Corporation), and 19.4 mL of a 3 M potassium phosphate aqueous solution purged with nitrogen gas were put, and the resulting mixture was heated and stirred in an oil bath at 50° C. for two hours to be caused to react. 120 mL of toluene was added to the reaction mixture, and the organic layer was washed with deionized water. After washing, the organic layer was dried over anhydrous magnesium sulfate. Thereafter, the insoluble matter was separated by filtration, and the filtrate was concentrated by a rotary evaporator to obtain a crude product. The obtained crude product was purified with a reversed-phase silica gel column (methanol/ethyl acetate=90/10 to 80/20) to obtain a compound (1-d) as a green solid. The obtained amount was 4.92 g (yield: 49%). A measurement result of an 1H-NMR spectrum of the compound (1-d) is as follows.

¹H-NMR (400 MHz, CDCl₃): δ (ppm)=9.72 (s, 1H), 7.62 (dd, 4H), 7.46-7.31 (m, 8H), 7.22 (s, 1H), 6.73 (s, 1H), 6.56 (d, 2H), 3.81 (t, 2H), 3.51 (t, 2H), 2.98 (s, 3H), 1.92-1.76 (m, 6H), 1.03 (s, 9H), 0.89 (d, 6H), 0.86 (d, 6H).

### Synthesis of Compound (1)

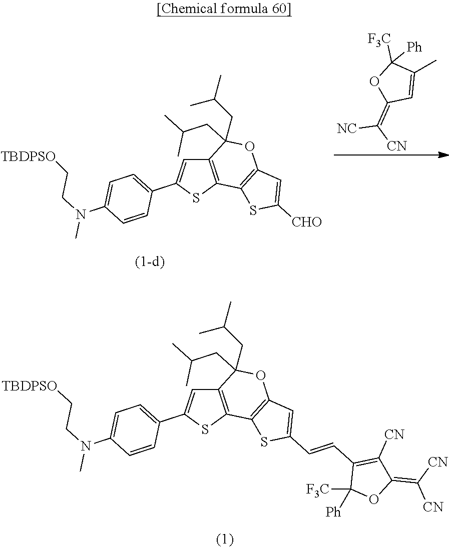

Into a 500 mL eggplant flask equipped with a three-way cock and a gas introduction tube, 4.92 g (6.81 mmol) of the synthesized compound (1-d), 2.58 g (8.18 mmol) of 2-(3-cyano-4-methyl-5-phenyl-5-(trifluoromethyl)-2 (5H)-furanylidene)-propanedinitrile (manufactured by iChemical), 64 mL of dehydrated ethanol, and 80 mL of dehydrated chloroform were put. A stirrer was further put thereinto, and the inside was replaced with nitrogen. Reaction was caused at room temperature (25° C.) for 30 hours while the reaction mixture was stirred with a magnetic stirrer. After completion of the reaction, the reaction mixture was concentrated by a rotary evaporator. Ethanol was added to the concentrate, the precipitate was separated by filtration, and the precipitate was further washed with ethanol to obtain a crude product. The crude product was purified with a reversed-phase silica gel column (acetonitrile/ethyl acetate=100/0 to 90/10) to obtain a compound (1) as a green solid. The obtained amount was 6.30 g (yield: 70%).

Measurement results of an 1H-NMR spectrum and a UV visible light spectrum of the compound (1) are as follows.

¹H-NMR (400 MHz, CDCl₃): δ (ppm)=7.61 (dd, 4H), 7.55-7.47 (m, 5H), 7.44-7.31 (m, 9H), 6.90 (s, 1H), 6.77 (s, 1H), 6.59-6.53 (m, 3H), 3.80 (t, 2H), 3.53 (t, 2H), 3.00 (s, 3H), 1.84-1.76 (m, 6H), 1.03 (s, 9H), 0.85 (d, 6H), 0.83 (d, 6H).

UV visible light spectrum: λmax=833 nm (in chloroform)

### (B) Calculation of Dipole Moment of Compound (1)

A dipole moment μ of the compound (1) was calculated by Gaussian 09 which is a quantum chemical calculation program manufactured by Gaussian. Structure optimization calculation was performed by pcm calculation (designating chloroform as a solvent) under an M062X/6-31+g(d) condition. The dipole moment μ of the compound (1) was 26 debye.

### (C) Calculation of Distance CN of Compound (1)

A structural formula was first drawn with ChemDraw with reference to a method described on page 31-32 of “Chem3D v15 User Guide (perkinelmer.co.jp” of PerkinElmer, Inc. Then, structure optimization calculation was performed by pcm calculation (designating chloroform as a solvent) under an M062X/6-31+g(d) condition, and then a distance CN of the compound (1) was calculated using Structure>Measurements>Display Distance Measurement on Chem3D, and found to be 13.1 Å.

### Example 1-2

### (A) Synthesis of Compound (2)

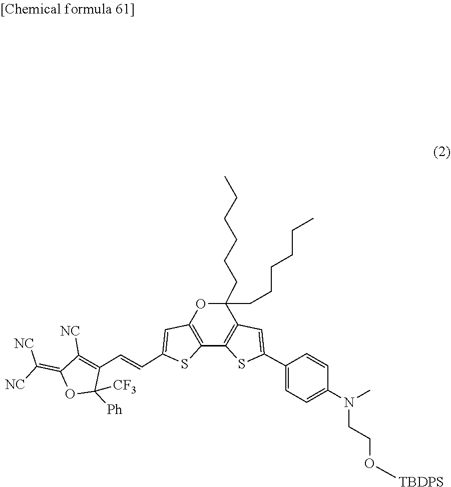

### Synthesis of Compound (2-b)

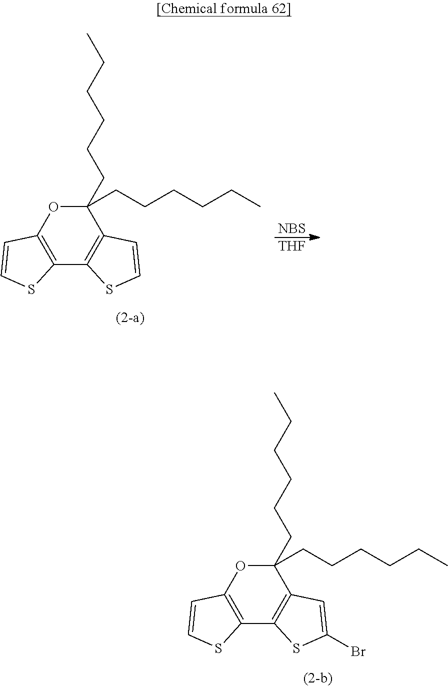

Into a 500 mL three-necked flask equipped with a Dimroth with a three-way cock at a top and a 100 mL equilibrium type dropping funnel, 10.00 g (27.6 mmol) of the compound (2-a) synthesized by a method described in WO 2013/047858 A and 100 mL of dehydrated tetrahydrofuran (THF, manufactured by FUJIFILM Wako Pure Chemical Corporation) were put. A stirrer was further put thereinto, and the inside was replaced with nitrogen. Thereafter, the flask containing the reaction mixture was immersed in a dry ice acetone bath, and the reaction mixture was cooled to −40° C. A solution adjusted by dissolving 5.16 g (29.0 mmol) of N-bromosuccinimide (manufactured by Tokyo Chemical Industry Co., Ltd.) (NBS) in 50 mL of dehydrated THF was put into the dropping funnel. A THF solution of NBS was slowly added dropwise from the dropping funnel such that the temperature of the reaction mixture did not exceed −40° C. After completion of the dropwise addition, stirring was continued at −40° C. for three hours. Thereafter, the flask was taken out of the dry ice acetone bath and further stirred at room temperature (25° C.) for 16 hours for reaction. After completion of the reaction, the reaction mixture was transferred to a 500 mL eggplant flask and concentrated by a rotary evaporator. 200 mL of chloroform and 200 mL of deionized water were added to the obtained concentrate, a product was extracted, and an organic layer was separated. The obtained organic layer was further washed with 100 mL of deionized water three times, and the separated organic layer was dried over anhydrous magnesium sulfate. Thereafter, the insoluble matter was separated by filtration, and the filtrate was concentrated and dried by a rotary evaporator to obtain the compound (2-b). The obtained amount was 10.93 g (yield: 90%). A measurement result of an ¹H-NMR spectrum of the compound (2-b) is as follows.

¹H-NMR (400 MHz, CD₃COCD₃): δ (ppm)=7.26 (d, 1H), 7.04 (s, 1H), 6.72 (d, 1H), 1.98-1.85 (m, 4H), 1.50-1.17 (m, 16H), 0.94-0.77 (m, 6H).

### Synthesis of Compound (2-c)

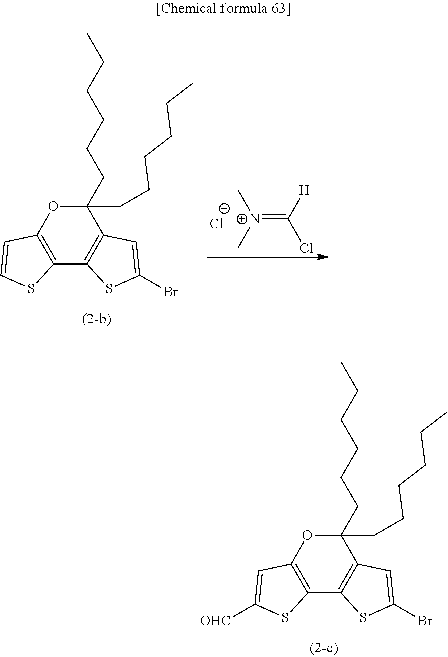

Into a 500 mL three-necked flask equipped with a three-way cock, 8.00 g (18.1 mmol) of the compound (2-b) and 150 mL of dehydrated chloroform (manufactured by FUJIFILM Wako Pure Chemical Corporation) were put. A stirrer was further put thereinto, and the inside was replaced with nitrogen. At room temperature (25° C.), 4.64 g (36.2 mmol) of a Vilsmeier reagent (manufactured by Tokyo Chemical Industry Co., Ltd.) was added thereto in three portions while the reaction mixture was stirred with a magnetic stirrer. After completion of the addition, the resulting mixture was further stirred for 24 hours to be caused to react. After completion of the reaction, 50 mL of deionized water was added thereto to quench the reaction. The organic layer was separated, then further washed with 50 mL of deionized water, and then dried over anhydrous magnesium sulfate. The insoluble matter was separated by filtration. The filtrate was transferred to a 500 mL eggplant flask and concentrated by a rotary evaporator. 200 mL of ethyl acetate and 200 mL of deionized water were added to the obtained concentrate, and a product was extracted. The contents of the eggplant flask were transferred to a 500 mL separating funnel to separate the organic layer, and the organic layer was further washed three times with 100 mL of deionized water. After washing, the organic layer was dried over anhydrous magnesium sulfate. Thereafter, the insoluble matter was separated by filtration, and the filtrate was concentrated and dried by a rotary evaporator to obtain a compound (2-c). The obtained amount was 6.40 g (yield: 75%). A measurement result of an ¹H-NMR spectrum of the compound (2-c) is as follows.

¹H-NMR (400 MHz, CD₃COCD₃): δ (ppm)=9.85 (s, 1H), 7.81, 7.55 (ss, 1H), 7.1 7, 6.95 (ss, 1H), 2.03-1.87 (m, 4H), 1.50-1.17 (m, 16H), 0.94-0.77 (m, 6H).

### Synthesis of Compound (2-d)

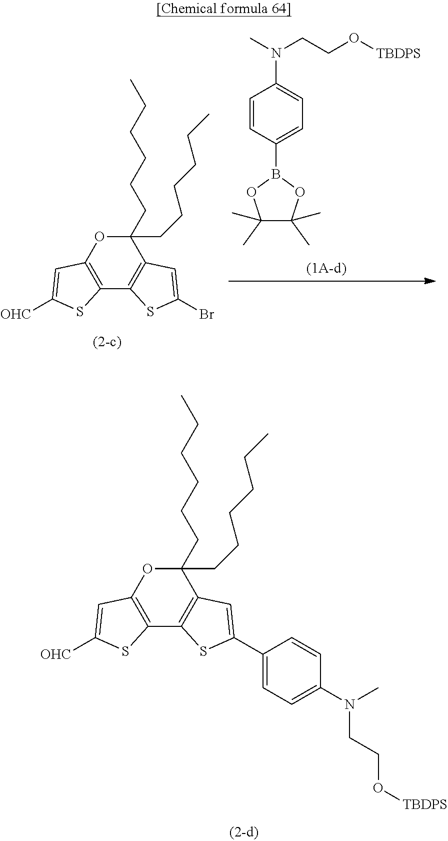

Into a 500 mL three-necked flask equipped with a three-way cock at a top and an induction type stirring blade, 3.00 g (6.39 mmol) of the compound (2-c), 4.94 g (9.58 mmol) of a compound (1A-d), and 200 mL of dehydrated THF were put, and the inside was replaced with argon. 0.18 g (0.13 mmol) of (tris(dibenzylideneacetone) dipalladium (0) (manufactured by Strem Chemicals) and 0.22 g (0.51 mmol) of tri-tert-butylphosphonium tetrafluoroborate (manufactured by Tokyo Chemical Industry Co., Ltd.) were added thereto while the reaction mixture was stirred, and 48 mL (144 mmol) of a 3 M potassium phosphate aqueous solution was further added thereto. The flask was immersed in an oil bath at 80° C., and reaction was caused for nine hours under reflux while the reaction mixture was vigorously stirred. After completion of the reaction, the reaction mixture was cooled to room temperature (25° C.), stirring was stopped, and the reaction mixture was allowed to stand still. The aqueous layer of the reaction mixture separated into two layers was removed, and the organic layer was dried over anhydrous magnesium sulfate. The insoluble matter was filtered. The filtrate was transferred to a 500 mL eggplant flask and concentrated by a rotary evaporator. The obtained concentrate was purified by column chromatography using toluene as an eluent to obtain the target compound (7-d). The obtained amount was 3.85 g (yield: 77%). A measurement result of an ¹H-NMR spectrum of the compound (2-d) is as follows.

¹H-NMR (400 MHz, CD₃COCD₃): δ (ppm)=9.82 (s, 1H), 7.73-7.64 (m, 4H), 7.55-7.35 (m, 8H), 6.75-6.68 (m, 2H), 3.86 (t, 2H), 3.65 (t, 2H), 2.81 (s, 3H), 2.03-1.87 (m, 4H), 1.50-1.17 (m, 16H), 1.03 (s, 9H), 0.94-0.77 (m, 6H).

### Synthesis of Compound (2)

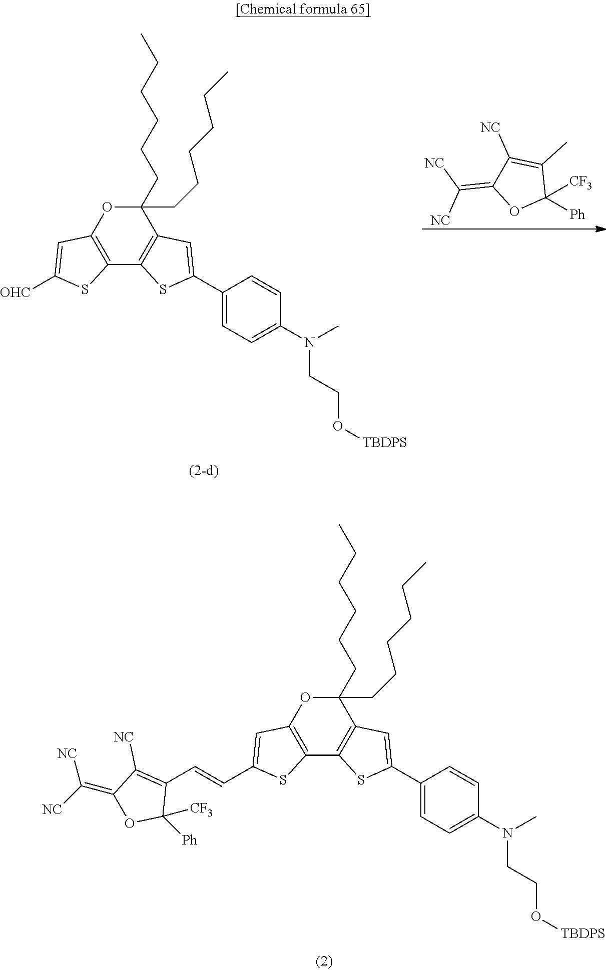

Into a 500 mL eggplant flask equipped with a three-way cock at a top, 3.85 g (4.95 mmol) of the compound (2-d), 1.87 g (5.94 mmol) of 2-(3-cyano-4-methyl-5-phenyl-5-(trifluoromethyl)-2(5H)-furanylidene)-propanedinitrile (manufactured by iChemical), 100 mL of dehydrated ethanol, and 100 mL of dehydrated chloroform were put. A stirrer was further put thereinto, and the inside was replaced with nitrogen. Reaction was caused at room temperature (25° C.) for 24 hours while the reaction mixture was stirred with a magnetic stirrer. After completion of the reaction, the reaction mixture was concentrated by a rotary evaporator. Ethanol was added to the concentrate, a precipitate was separated by filtration, and the precipitate was further washed with methanol to obtain a compound (2) as a blue solid. The obtained amount was 4.55 g (yield: 86%). A measurement result of an ¹H-NMR spectrum of the compound (2) is as follows.

¹H-NMRR (400 MHz, CD₃COCD₃): δ (ppm)=7.86-7.34 (m, 18H), 6.85-6.69 (m, 3H), 3.86 (t, 2H), 3.65 (t, 2H), 2.81 (s, 3H), 2.03-1.87 (m, 4H), 1.50-1.17 (m, 16H), 1.03 (s, 9H), 0.94-0.77 (m, 6H).

UV visible light spectrum: λmax=835 nm (in chloroform)

### (B) Calculation of Dipole Moment of Compound (2)

Structure optimization calculation was performed as in the compound (1). A dipole moment μ of the compound (2) was 26 debye.

### (C) Calculation of Distance CN of Compound (2)

Structure optimization calculation was performed as in the compound (1), and a distance CN of the compound (2) was calculated and found to be 13.1 Å.

### Comparative Example 1-1

### (A) Synthesis of Compound (3)

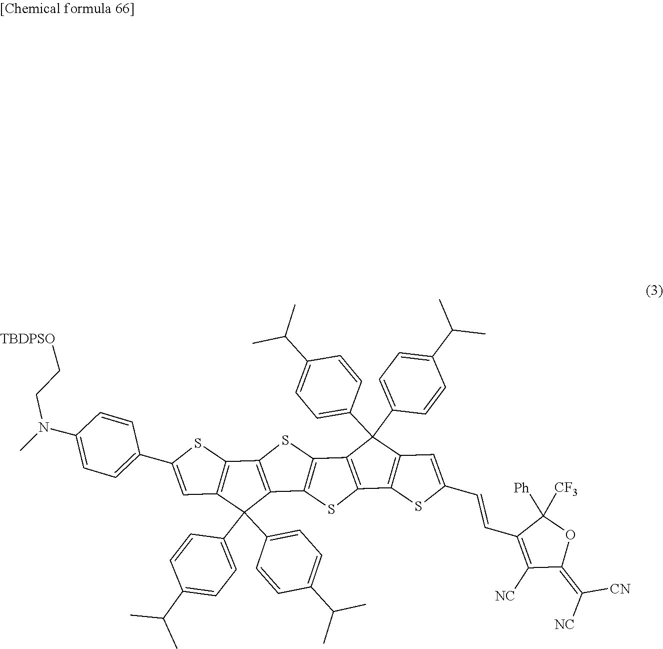

### Synthesis of Compound (3-b)

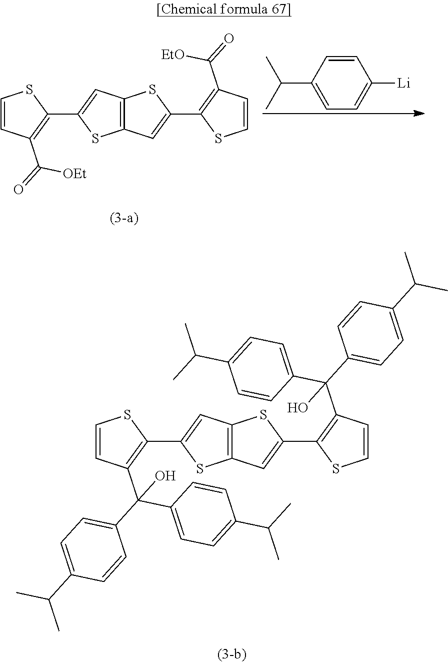

A compound (3-a) was synthesized by a method described in J. Mater. Chem. C, 2016, 4, 9656-9663. Into a 200 mL eggplant flask equipped with a three-way cock and a gas introduction tube, 8.43 g (42.4 mmol) of 4-bromocumene (manufactured by Aldrich) and 38 mL of dehydrated tetrahydrofuran (manufactured by KANTO CHEMICAL CO., INC.) were put. A stirrer was further put thereinto, the inside was replaced with nitrogen, and the solution was cooled to −60° C. 26 mL (41.5 mmol) of a 1.6 M n-butyllithium-hexane solution (manufactured by KANTO CHEMICAL CO., INC.) was added dropwise thereto, and then the resulting mixture was caused to react at −60° C. for 30 minutes while being stirred with a magnetic stirrer. 3.80 g (8.47 mmol) of the compound (3-a) was added to the reaction solution, and then the temperature was raised to 0° C. over one hour. 57 mL of methanol was added to the reaction mixture. Thereafter, the insoluble matter was separated by filtration, and the filtrate was concentrated by a rotary evaporator to obtain a compound (3-b) as a pale yellow solid. The obtained amount was 5.51 g (yield: 78%). A measurement result of an ¹H-NMR spectrum of the compound (3-b) is as follows.

¹H-NMR (400 MHz, CDCl₃): δ (ppm)=7.17-7.09 (m, 18H), 6.57 (s, 2H), 6.50 (d, 2H), 3.18 (s, 2H), 2.87 (sept, 4H), 1.22 (d, 24H).

### Synthesis of Compound (3-c)

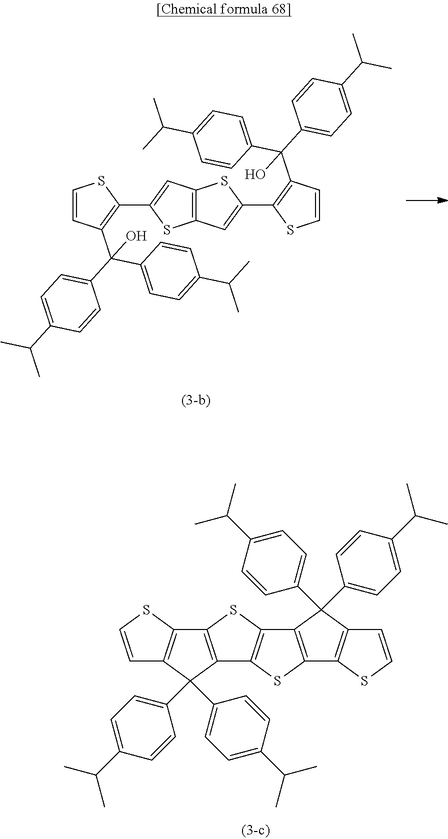

Into a 500 mL eggplant flask equipped with a Dimroth with a three-way cock at a top and a gas introduction tube, 6.08 g (7.17 mmol) of the synthesized compound (3-b) and 240 mL of toluene (manufactured by KANTO CHEMICAL CO., INC.) were put. A stirrer was further put thereinto, and the inside was purged with nitrogen.

1.53 g (10.8 mmol) of a boron trifluoride-diethyl ether complex was added thereto, and the resulting mixture was caused to react under reflux condition for one hour while being stirred with a magnetic stirrer. The obtained reaction mixture was washed with deionized water. After washing, the organic layer was dried over anhydrous magnesium sulfate. Thereafter, the insoluble matter was separated by filtration, and the filtrate was concentrated by a rotary evaporator. The concentrate was washed with acetonitrile to obtain a compound (3-c) as a red solid. The obtained amount was 4.93 g (yield: 86%). A measurement result of an ¹H-NMR spectrum of the compound (3-c) is as follows.

¹H-NMR (400 MHz, CDCl₃): δ (ppm)=7.18-7.08 (m, 18H), 7.07 (s, 2H), 2.84 (sept, 4H), 1.25 (d, 24H).

### Synthesis of Compound (3-d)

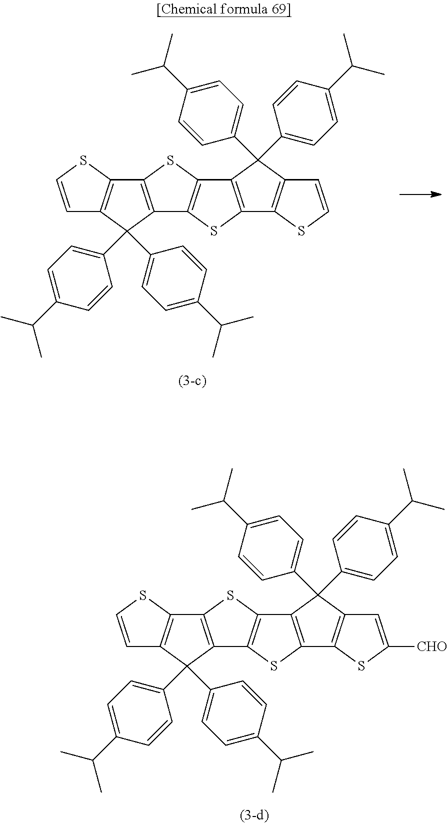

Into a 500 mL eggplant flask equipped with a Dimroth with a three-way cock at a top and a gas introduction tube, 4.76 g (5.94 mmol) of the synthesized compound (3-c), 71 mL of dehydrated tetrahydrofuran (manufactured by KANTO CHEMICAL CO., INC.), and 71 mL of dehydrated dimethylformamide (manufactured by KANTO CHEMICAL CO., INC.) were put. A stirrer was further put thereinto, and the inside was replaced with nitrogen. 1.14 g (8.91 mmol) of (chloromethylene) dimethyliminium chloride (manufactured by Tokyo Chemical Industry Co., Ltd.) was added thereto, and the resulting mixture was caused to react at 25° C. for nine hours while being stirred with a magnetic stirrer. 238 mL of toluene was added to the reaction mixture, and the organic layer was washed with deionized water. After washing, the organic layer was dried over anhydrous magnesium sulfate. Thereafter, the insoluble matter was separated by filtration, and the filtrate was concentrated by a rotary evaporator to obtain a compound (3-d) as a brown solid. The obtained amount was 5.21 g (yield: 105%).

### Synthesis of Compound (3-e)

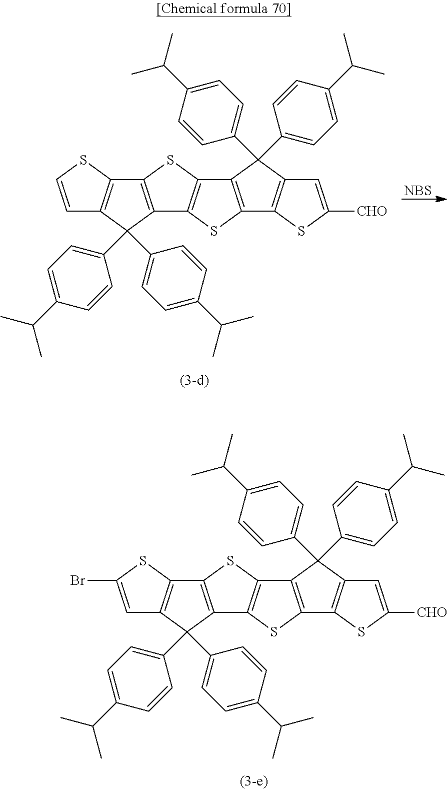

Into a 200 mL eggplant flask equipped with a three-way cock and a gas introduction tube, 5.20 g (5.16 mmol) of the synthesized compound (3-d) and 78 mL of dehydrated tetrahydrofuran (manufactured by FUJIFILM Wako Pure Chemical Corporation) were put. A stirrer was further put thereinto, the inside was replaced with nitrogen, and the solution was cooled to 0° C. 22.7 g (128 mmol) of N-bromosuccinimide (manufactured by Tokyo Chemical Industry Co., Ltd.) was added thereto, and then the resulting mixture was caused to react at 0° C. for one hour while being stirred with a magnetic stirrer. 47 mL of toluene was added to the reaction mixture, and the organic layer was washed with deionized water. After washing, the organic layer was dried over anhydrous magnesium sulfate. Thereafter, the insoluble matter was separated by filtration, and the filtrate was concentrated by a rotary evaporator to obtain a crude product. The obtained crude product was purified with a silica gel column (toluene/ethyl acetate=100/0 to 90/10), and then washed with methanol to obtain a compound (3-e) as a red solid. The obtained amount was 2.20 g (yield: 47%). A measurement result of an ¹H-NMR spectrum of the compound (3-e) is as follows.

¹H-NMR (400 MHz, CDCl₃): δ (ppm)=9.76 (s, 1H), 7.65 (s, 1H), 7.17-7.10 (m, 16H), 7.08 (s, 1H), 7.07 (s, 2H), 2.86 (sept, 4H), 1.21 (d, 24H).

### Synthesis of Compound (3-f)

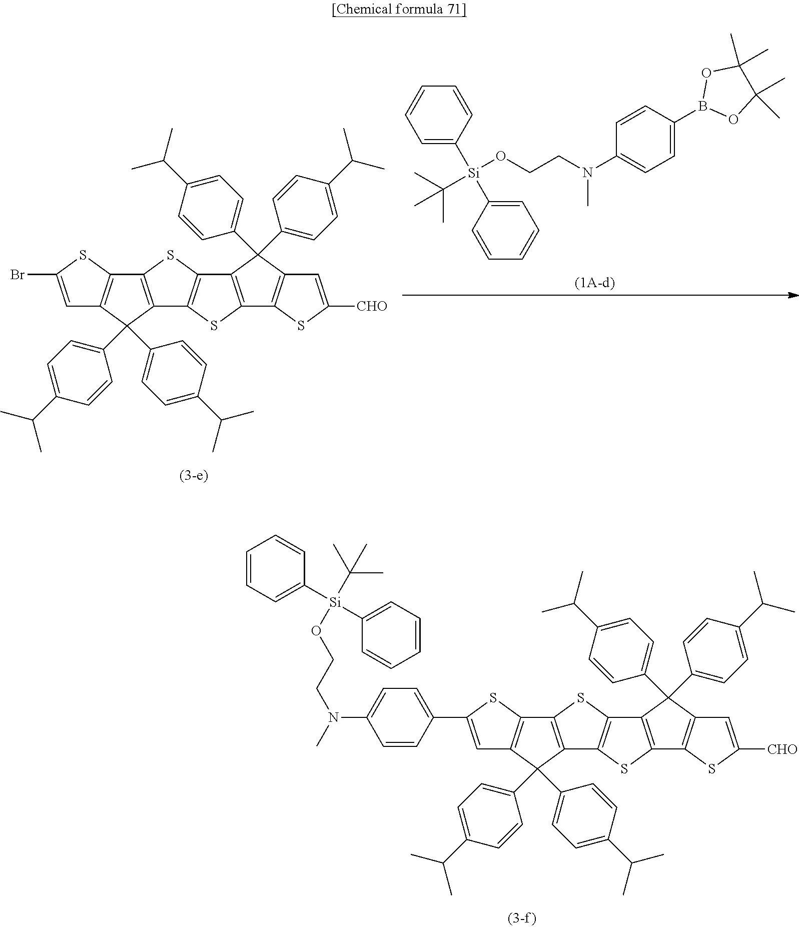

Into a 300 mL four-necked flask equipped with a Dimroth with a three-way cock at a top and a gas introduction tube, 0.55 g (0.96 mmol) of the synthesized compound (3-e), 0.59 g (1.15 mmol) of the compound (1A-d), and 90 mL of dehydrated tetrahydrofuran (manufactured by FUJIFILM Wako Pure Chemical Corporation) were put. A stirrer was further put thereinto, and the inside was replaced with nitrogen. 0.03 g (0.024 mmol) of tris(dibenzylideneacetone) dipalladium (0) (manufactured by N.E. CHEMCAT CORPORATION), 0.03 g (0.096 mmol) of tri-tert-butylphosphonium tetrafluoroborate (manufactured by FUJIFILM Wako Pure Chemical Corporation), and 1.3 mL of a 3 M potassium phosphate aqueous solution purged with nitrogen gas were put, and the resulting mixture was heated and stirred in an oil bath at 50° C. for 1.5 hours to be caused to react. 28 mL of toluene was added to the reaction mixture, and the organic layer was washed with deionized water. After washing, the organic layer was dried over anhydrous magnesium sulfate. Thereafter, the insoluble matter was separated by filtration, and the filtrate was concentrated by a rotary evaporator to obtain a crude product. The obtained crude product was washed with acetonitrile to obtain a compound (3-f) as a red solid. The obtained amount was 0.70 g (yield: 101%).

### Synthesis of Compound (3)

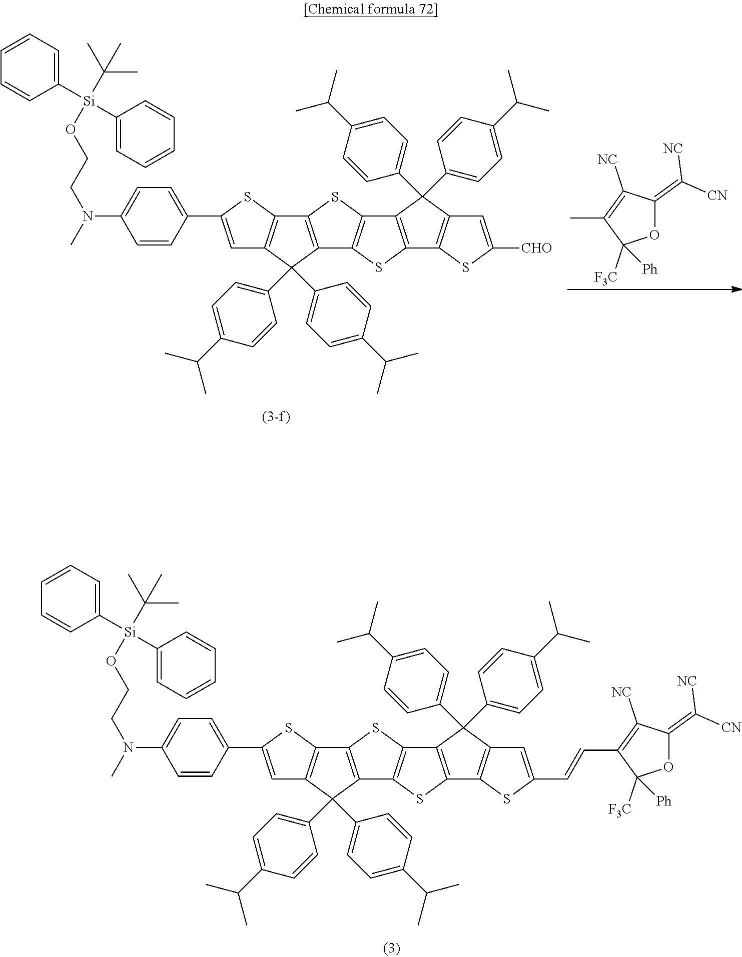 [SMILES: CC(C)c1ccc(C2(c3ccc(C(C)C)cc3)c3cc(C=O)sc3-c3sc4c5c(sc4c32)-c2sc(-c3ccc(N(C)CCO[Si](c4ccccc4)(c4ccccc4)C(C)(C)C)cc3)cc2C5(c2ccc(C(C)C)cc2)c2ccc(C(C)C)cc2)cc1 ; CC1=C(C#N)C(=C(C#N)C#N)OC1(c1ccccc1)C(F)(F)F ; CC(C)c1ccc(C2(c3ccc(C(C)C)cc3)c3cc(/C=C/C4=C(C#N)C(=C(C#N)C#N)OC4(c4ccccc4)C(F)(F)F)sc3-c3sc4c5c(sc4c32)-c2sc(-c3ccc(N(C)CCO[Si](c4ccccc4)(c4ccccc4)C(C)(C)C)cc3)cc2C5(c2ccc(C(C)C)cc2)c2ccc(C(C)C)cc2)cc1]

Into a 100 mL eggplant flask equipped with a three-way cock and a gas introduction tube, 0.54 g (0.44 mmol) of the synthesized compound (3-f), 0.34 g (1.07 mmol) of 2-(3-cyano-4-methyl-5-phenyl-5-(trifluoromethyl)-2 (5H)-furanylidene)-propanedinitrile (manufactured by iChemical), 11 mL of dehydrated ethanol, and 11 mL of dehydrated chloroform were put. A stirrer was further put thereinto, and the inside was replaced with nitrogen. Reaction was caused at room temperature (25° C.) for 24 hours while the reaction mixture was stirred with a magnetic stirrer. After completion of the reaction, the reaction mixture was concentrated by a rotary evaporator to obtain a crude product. The crude product was purified with a reversed-phase silica gel column (acetonitrile/ethyl acetate=80/20) and then washed with ethyl acetate and hexane to obtain a compound (3) as a green solid. The obtained amount was 0.36 g (yield: 54%). The compound (3) has a divalent polycyclic condensed ring group having two or more thiophene rings and having an sp3 carbon atom as a constituent element, and further has a group represented by formula (b1). Measurement results of an 1H-NMR spectrum and a UV visible light spectrum of the compound (3) are as follows.

¹H-NMR (400 MHz, CDCl₃): δ (ppm)=7.61 (dd, 4H), 7.55-7.47 (m, 5H), 7.44-7.31 (m, 9H), 6.90 (s, 1H), 6.77 (s, 1H), 6.59-6.53 (m, 3H), 3.80 (t, 2H), 3.53 (t, 2H), 3.00 (s, 3H), 1.84-1.76 (m, 6H), 1.03 (s, 9H), 0.85 (d, 6H), 0.83 (d, 6H).

UV visible light spectrum: λmax=879 nm (in chloroform)

### (B) Calculation of Dipole Moment of Compound (3)

Structure optimization calculation was performed as in the compound (1). A dipole moment μ of the compound (3) was 28 debye.

### (C) Calculation of Distance CN of Compound (3)

Structure optimization calculation was performed as in the compound (1), and a distance CN of the compound (3) was calculated and found to be 19.2 Å.

### 2. Preparation of EO Film and Evaluation of Absorbance

### (Example 2-1) Preparation of EO Film Containing Compound (1) and Evaluation of Absorbance

### (A) Preparation of EO Film

A coating solution was prepared by adjusting a mass ratio of the compound (1) and polymethyl methacrylate (PMMA) to be 2:8. The coating solution was applied to a washed substrate (ITO-coated glass, quartz glass) at a speed of 500 to 3000 rpm using a spin coater MS-A100 (manufactured by Mikasa Co., Ltd.), and then vacuum-dried for one hour around a glass transition temperature (Tg).

As a result, an EO film of Example 2-1 having a film thickness of 880 nm was obtained.

### (B) Measurement of UV Visible Light Spectrum

A UV visible light spectrum was measured using the EO film of Example 2-1. A maximum absorption wavelength (λmax) was 802 nm, and a long wavelength side half-value width was 116 nm.

Absorbance at 1262 nm in the O band region is extremely low, and actual measurement is difficult. Therefore, absorbance prediction at 1262 nm was performed assuming that a normal distribution was obtained as a peak shape. Absorbance at the absolute maximum (maximum) absorption wavelength was normalized to 1 and fitted with a Gaussian function, and the absorbance at 1262 nm was calculated and found to be 2.0×10⁻⁵.

### (Example 2-2) Preparation of EO Film Containing Compound (2) and Evaluation of Absorbance

### (A) Preparation of EO Film

An EO film of Example 2-2 having a film thickness of 1071 nm was obtained in a similar manner to Example 2-1 except that the compound (1) was changed to the compound (2).

### (B) Measurement of UV Visible Light Spectrum

A UV visible light spectrum was measured using the EO film of Example 2-2. A maximum absorption wavelength (λmax) was 809 nm, and a long wavelength side half-value width was 112 nm.

Absorbance at 1262 nm was calculated in a similar manner to Example 2-1, and found to be 1.3×10⁻⁵.

### (Comparative Example 2-1) Preparation of EO Film Containing Compound (3) and Evaluation of Absorbance

### (A) Preparation of EO Film

An EO film of Comparative Example 2-1 having a film thickness of 880 nm was obtained in a similar manner to Example 2-1 except that the compound (1) was changed to the compound (3).

### (B) Measurement of UV Visible Light Spectrum

A UV visible light spectrum was measured using the EO film of Comparative Example 2-1. A maximum absorption wavelength (λmax) was 842 nm, and a long wavelength side half-value width was 125 nm.

Absorbance at 1262 nm was calculated in a similar manner to Example 2-1, and found to be 4.3×10⁻⁴.

TABLE 1

| | | | | | Long | |
|---|---|---|---|---|---|---|
| | EO compound | | | Maximum | wavelength | |
| | | Dipole | | absorption | side half- | Absorbance |
| | | moment | Distance | wavelength | value | (prediction) |
| EO film | Type | (debye) | CN (Å) | λmax (nm) | width (nm) | at 1262 nm |
| Example 2-1 | Compound | 26 | 13.1 | 802 | 116 | 2.0 × 10⁻⁵ |
| | (1) | | | | | |
| Example 2-2 | Compound | 26 | 13.1 | 809 | 112 | 1.3 × 10⁻⁵ |
| | (2) | | | | | |
| Comparative | Compound | 28 | 19.2 | 842 | 125 | 4.3 × 10⁻⁴ |
| Example 2-1 | (3) | | | | | |

As presented in Table 1, it has been found that the maximum absorption wavelength and the long wavelength side half-value width of the EO film affect the absorbance at 1262 nm. More specifically, it has been found that when the maximum absorption wavelength (λmax) of the EO film is 760 to 830 nm and the long wavelength side half-value width of the EO film is 90 to 120 nm, the absorbance at 1262 nm tends to be lower than that when such conditions are not satisfied.

### 3. Preparation and Evaluation of EO Film

### (Example 3-1) Preparation and Evaluation of EO Film Containing Compound (1)

### (A) Preparation of EO Film

EO films of Example 3-1, which are compound (1)/PMMA mixed films (content of compound (1): 20 mass %), were obtained on quartz glass in 2 types of film thicknesses of 3 μm and 0.1 μm, respectively.

### (B) Calculation of Optical Loss of EO Film

An optical loss was calculated using the EO films of Example 3-1 having different film thicknesses. Each quartz glass was placed on an aluminum substrate, and an absorption spectrum was measured using a spectrophotometer Cary 5000 (manufactured by Agilent) and Near-Normal Specular Reflection Accessory (manufactured by HARRICK). An optical loss α of the electro-optic film was calculated from a slope of a straight line obtained by plotting absorbance for each film thickness at a wavelength of 1260 nm. The optical loss α of the EO film of Example 3-1 was 16.0 dB/cm.

### (C) Calculation of EO Coefficient of EO Film

Using the EO film of Example 3-1, an EO coefficient r33 was measured in a similar manner to a method described in a reference paper (“Transmission ellipsometric method without an aperture for simple and reliable evaluation of electro-optic properties”, Toshiki Yamada and Akira Otomo, Optics Express, vol. 21, pages 29240-48 (2013)). As laser light sources, LP1310-SAD2 (1310 nm) and LP1550-SAD2 (1550 nm) of semiconductor DFB laser (manufactured by THORLABS) were used. The EO coefficient r33 of the EO film of Example 3-1 at 1310 nm was 71.6 μm/V. A ratio of the EO coefficient r33 at 1310 nm to the optical loss α (index r33/α) of the EO film of Example 3-1 was 4.5.

### (Example 3-2) Preparation and Evaluation of EO Film Containing Compound (2)

### (A) Preparation of EO Film

An EO film of Example 3-2 was obtained in a similar manner to Example 3-1 except that the compound (1) was changed to the compound (2).

### (B) Calculation of EO Coefficient of EO Film

Using the EO film of Example 3-2, an optical loss of the EO film was calculated in a similar manner to Example 3-1.

The optical loss α of the EO film of Example 3-2 was 32.9 dB/cm.

### (C) Calculation of EO Coefficient of EO Film

Using the EO film of Example 3-2, an EO coefficient of the EO film was calculated in a similar manner to Example 3-1. The EO coefficient r33 of the EO film of Example 3-2 at 1310 nm was 52.6 μm/V. A ratio of the EO coefficient r33 at 1310 nm to the optical loss α (index r33/α) of the EO film of Example 3-2 was 1.6.

### (Comparative Example 3-1) Preparation and Evaluation of EO Film Containing Compound (3)

### (A) Preparation of EO Film

An EO film of Comparative Example 3-1 was obtained in a similar manner to Example 3-1 except that the compound (1) was changed to the compound (3).

### (B) Calculation of Optical Loss of EO Film

Using the EO film of Comparative Example 3-1, an optical loss of the EO film was calculated in a similar manner to Example 3-1.

The optical loss α of the EO film of Comparative Example 3-1 was 166.7 dB/cm.

### (C) Calculation of EO Coefficient of EO Film

Using the EO film of Example 3-1, an EO coefficient of the EO film was calculated in a similar manner to Comparative Example 3-1. The EO coefficient r33 of the EO film of Comparative Example 3-1 at 1310 nm was 48.8 μm/V. A ratio of the EO coefficient r33 at 1310 nm to the optical loss α (index r33/α) of the EO film of Comparative Example 3-1 was 0.29.

TABLE 2

| | | Optical | EO coef- | |
|---|---|---|---|---|
| | | loss α | ficient | |
| | Type of EO | (dB/cm) at | r33 (pm/V) at | |
| EO film | compound | 1260 nm | 1310 nm | Index r33/α |
| Example 3-1 | Compound (1) | 16.0 | 71.6 | 4.5 |
| Example 3-2 | Compound (2) | 32.9 | 52.6 | 1.6 |
| Comparative | Compound (3) | 167.7 | 48.8 | 0.29 |
| Example 3-1 | | | | |

As presented in Table 2, it has been found that the EO films of Examples 3-1 and 3-2 tend to have a smaller optical loss α in the O band region, a larger EO coefficient, and a larger index r33/α than the EO film of Comparative Example 3-1. From the above, it has been confirmed that the electro-optic film of the present invention can suppress an optical loss in the O band region (wavelength: 1260 to 1360 nm). The electro-optic film has a small optical loss α to an electro-optic coefficient r33 in the O band region (that is, has a large index r33/α), and therefore it can be said that the electro-optic film is suitable for optical communication in the O band region.

1. An electro-optic film comprising a compound (A) having a dipole moment of 19 to 31 debye, wherein
a maximum absorption wavelength (λmax) is 760 to 830 nm, and
a difference between the maximum absorption wavelength and an absorption wavelength on a longer wavelength side than the maximum absorption wavelength among absorption wavelengths exhibiting absorbance of a half value of maximum absorbance is 90 to 120 nm.

2. The electro-optic film according to claim 1, wherein the compound (A) is a compound represented by the following formula (1): 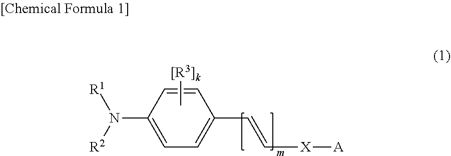
[in formula (1), X represents a bicyclic to pentacyclic divalent condensed ring group having two or more thiophene rings, and the divalent condensed ring group may have a substituent;
R¹ and R² each independently represent an alkyl group, a haloalkyl group, an acyloxyalkyl group, a trialkylsilyloxyalkyl group, an aryldialkylsilyloxyalkyl group, an alkyldiarylsilyloxyalkyl group, an aryl group, —R²¹—OH (R²¹ represents a divalent hydrocarbon group), —R²²—NH₂ (R²² represents a divalent hydrocarbon group), —R²³—SH (R²³ represents a divalent hydrocarbon group), or —R²⁴—NCO (R²⁴ represents a divalent hydrocarbon group), these groups may each have a crosslinkable group, and R¹ and R² may be bonded to each other to form a ring together with atoms to which R¹ and R² are bonded;
R³ represents an alkyl group, an alkyloxy group, an aryl group, an aryloxy group, an aralkyloxy group, a trialkylsilyloxyalkyl group, an aryldialkylsilyloxyalkyl group, an alkyldiarylsilyloxyalkyl group, an alkenyloxy group, an alkynyloxy group, a hydroxy group, an amino group, a sulfanyl group, an isocyanate group, —R³¹—OH (R³¹ represents a divalent hydrocarbon group), —O—R³²—OH (R³² represents a divalent hydrocarbon group), —R³³—NH₂ (R³³ represents a divalent hydrocarbon group), —R³⁴—SH (R³⁴ represents a divalent hydrocarbon group), —R³⁵—NCO (R³⁵ represents a divalent hydrocarbon group), or —OC(═O)R⁴¹ (R⁴¹ represents a monovalent hydrocarbon group), these groups may each have a crosslinkable group, when there is a plurality of R³s, R³s may be the same as or different from each other, and R³ may be bonded to R or R² to form a ring together with atoms to which R³ and R¹ or R² are bonded;
k represents an integer of 0 to 4;
A represents any one of groups represented by the following formulas (a1), (a2), and (b1): 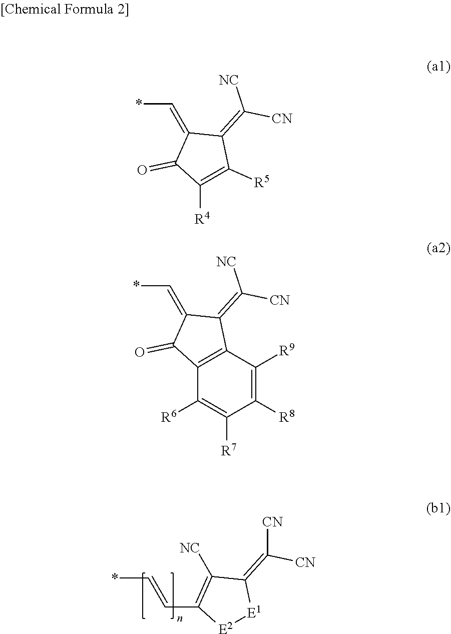
R⁴, R⁵, R⁶, R⁷, R⁸, and R⁹ each independently represent a hydrogen atom, a halogen atom, an alkyl group, a haloalkyl group, a cyano group, an aryl group, or a haloaryl group;
E¹ and E² each independently represent —C(R¹⁰)(R¹¹)—, —C(O)—, —O—, or —NR¹²—, note that at least one of E¹ and E² is —O— or —N¹²—, R¹⁰ and R¹¹ each independently represent a hydrogen atom, an alkyl group, a haloalkyl group, an aryl group, or a haloaryl group, and R¹² represents a hydrogen atom or an alkyl group;
when A is a group represented by formula (a1) or (a2), m is 0; and
when A is a group represented by formula (b1), m and n each independently represent 0 or 1, and note that m+n=1].

3. The electro-optic film according to claim 2, wherein the compound represented by the formula (1) is a compound represented by the following formula (2a2) or (2b1): 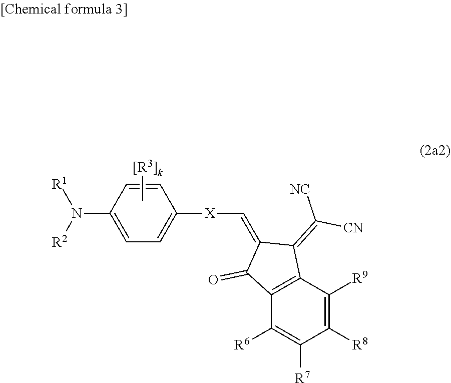
[in formula (2a2), X, R¹, R², R³, R⁶, R⁷, R⁸, R⁹, and k have the same meanings as those described above]; and 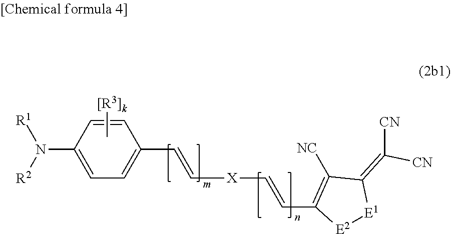
[in formula (2b1), X, R¹, R², R³, E¹, E², k, m, and n have the same meanings as those described above].

4. The electro-optic film according to claim 2, wherein the compound represented by the formula (1) is a compound represented by the following formula (2b1-1): 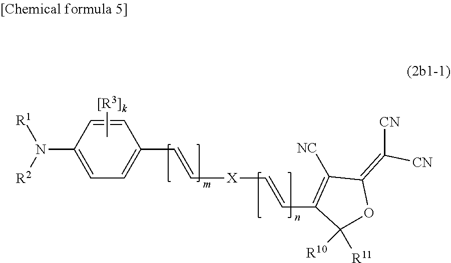
[in formula (2b1-1), X, R¹, R², R³, k, m, and n have the same meanings as those described above; and
R¹⁰ and R¹¹ each independently represent a methyl group, a trifluoromethyl group, a phenyl group, or a pentafluorophenyl group].

5. The electro-optic film according to claim 2, wherein the compound represented by the formula (1) is a compound represented by the following formula (5): 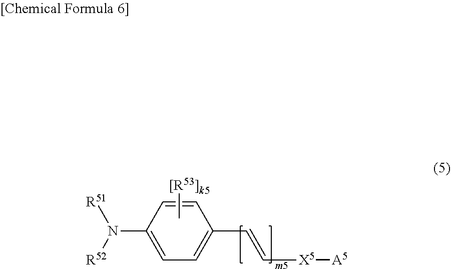
[in formula (5), X⁵ represents a bicyclic to tetracyclic divalent condensed ring group having two or more thiophene rings, and the divalent condensed ring group may have a substituent;
R⁵¹ and R⁵² each independently represent an alkyl group, a haloalkyl group, an acyloxyalkyl group, a trialkylsilyloxyalkyl group, an aryldialkylsilyloxyalkyl group, an alkyldiarylsilyloxyalkyl group, an aryl group, —R⁷¹—OH (R⁷¹ represents a divalent hydrocarbon group), —R⁷²—NH₂ (R⁷² represents a divalent hydrocarbon group), —R⁷³—SH (R⁷³ represents a divalent hydrocarbon group), or —R⁷⁴—NCO (R⁷⁴ represents a divalent hydrocarbon group), these groups may each have a crosslinkable group, and R⁵¹ and R⁵² may be bonded to each other to form a ring together with atoms to which R⁵¹ and R⁵² are bonded;
R⁵³ represents an alkyl group or an aryl group, these groups may each have a crosslinkable group, when there is a plurality of R⁵³s, R⁵³s may be the same as or different from each other, and R⁵³ may be bonded to R⁵¹ or R⁵² to form a ring together with atoms to which R⁵³ and R⁵¹ or R⁵² are bonded;
k5 represents an integer of 0 to 4;
A⁵ represents any one of groups represented by the following formulas (a51), (a52), and (b51): 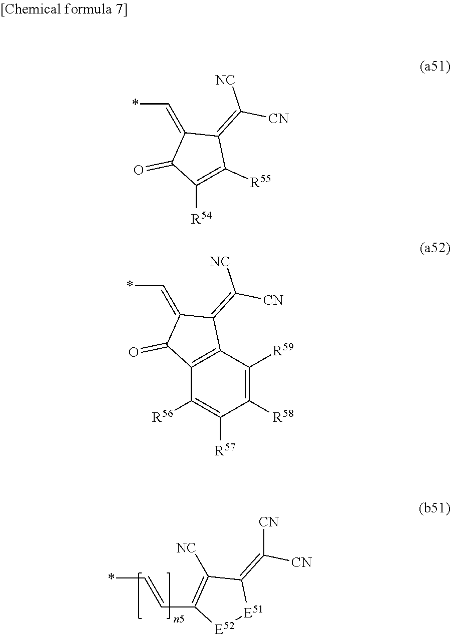
R⁵⁴, R⁵⁵, R⁵⁶, R⁵⁷, R⁵⁸, and R⁵⁹ each independently represent a hydrogen atom, a halogen atom, an alkyl group, a haloalkyl group, a cyano group, an aryl group, or a haloaryl group;
E⁵¹ and E⁵² each independently represent —C(R⁶⁰)(R⁶¹)—, —C(O)—, —O—, or —NR⁶²—, note that at least one of E⁵¹ and E⁵² is —O— or —NR⁶²—, R⁶⁰ and R⁶¹ each independently represent a hydrogen atom, an alkyl group, a haloalkyl group, an aryl group, or a haloaryl group, and R⁶² represents a hydrogen atom or an alkyl group;
when A⁵ is a group represented by formula (a51) or (a52), m5 is 0; and
when A⁵ is a group represented by formula (b51), m5 and n5 each independently represent 0 or 1, and note that m5+n5=1].

6. A compound represented by the following formula (5): 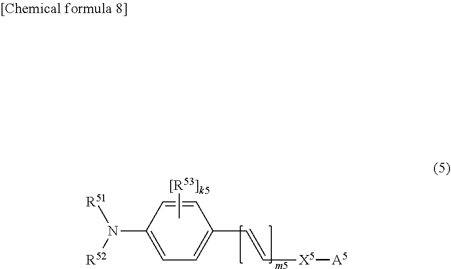
[in formula (5), X⁵ represents a bicyclic to tetracyclic divalent condensed ring group having two or more thiophene rings, and the divalent condensed ring group may have a substituent;
R⁵¹ and R⁵² each independently represent an alkyl group, a haloalkyl group, an acyloxyalkyl group, a trialkylsilyloxyalkyl group, an aryldialkylsilyloxyalkyl group, an alkyldiarylsilyloxyalkyl group, an aryl group, —R⁷¹—OH (R⁷¹ represents a divalent hydrocarbon group), —R⁷²—NH₂ (R⁷² represents a divalent hydrocarbon group), —R⁷³—SH (R⁷³ represents a divalent hydrocarbon group), or —R⁷⁴—NCO (R⁷⁴ represents a divalent hydrocarbon group), these groups may each have a crosslinkable group, and R⁵¹ and R⁵² may be bonded to each other to form a ring together with atoms to which R⁵¹ and R⁵² are bonded;
R⁵³ represents an alkyl group or an aryl group, these groups may each have a crosslinkable group, when there is a plurality of R⁵³s, R⁵³s may be the same as or different from each other, and R⁵³ may be bonded to R⁵¹ or R⁵² to form a ring together with atoms to which R⁵³ and R⁵¹ or R⁵² are bonded;
k5 represents an integer of 0 to 4;
A⁵ represents any one of groups represented by the following formulas (a51), (a52), and (b51): 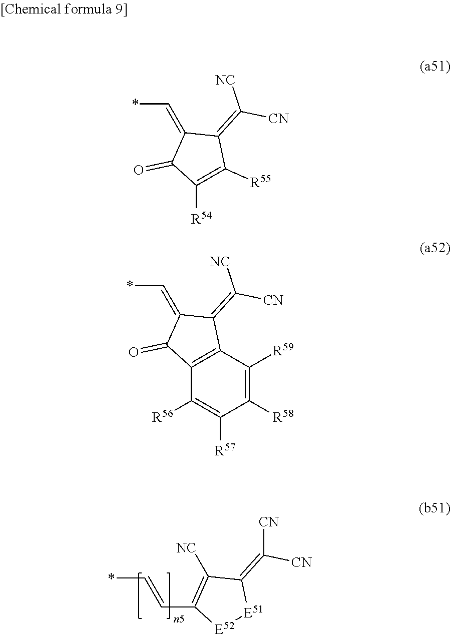
R⁵⁴, R⁵⁵, R⁵⁶, R⁵⁷, R⁵⁸, and R⁵⁹ each independently represent a hydrogen atom, a halogen atom, an alkyl group, a haloalkyl group, a cyano group, an aryl group, or a haloaryl group;
E⁵¹ and E⁵² each independently represent —C(R⁶⁰)(R⁶¹)—, —C(O)—, —O—, or —NR⁶²—, note that at least one of E⁵¹ and E⁵² is —O— or —NR⁶²—, R⁶⁰ and R⁶¹ each independently represent a hydrogen atom, an alkyl group, a haloalkyl group, an aryl group, or a haloaryl group, and R⁶² represents a hydrogen atom or an alkyl group;
when A⁵ is a group represented by formula (a51) or (a52), m5 is 0; and
when A⁵ is a group represented by formula (b51), m5 and n5 each independently represent 0 or 1, and note that m5+n5=1].

7. An electro-optic composition comprising the compound according to claim 6.

8. An electro-optic film comprising the compound according to claim 6.

9. An electro-optic element comprising the electro-optic film according to claim 1.
